(19) United States
(12) Patent Application Publication
(10) Pub. No.: US 2003/0207878 A1
(43) Pub. Date: Nov. 6, 2003

(54) Chemical compounds

(76) Inventor: Lawrent Francois Andre Hennequin, Macclesfield Cheshire (GB)

Correspondence Address: MORGAN LEWIS & BOCKIUS LLP, 1111 PENNSYLVANIA AVENUE NW, WASHINGTON, DC, 20004, US

(21) Appl. No.: 10/343,236

(22) PCT Filed: Aug. 8, 2001
(86) PCT No.: PCT/GB01/03561

(30) Foreign Application Priority Data
Aug. 9, 2000 (EP) 00402257.0

(51) Int. Cl. A61K 31/541; A61K 31/5377; A61K 31/53; A61K 31/496; C07D 417/14; C07D 413/14; C 07D 4 3/14

(52) U.S. Cl. 514/228.2; 514/234.5; 514/252.16; 514/262.1; 514/303; 544/60; 544/118; 544/184; 544/262

(57) ABSTRACT

The invention relates to compounds of the formula (I): wherein: ring C is 9 or 10-membered bicyclic heteroaromatic group containing at least one nitrogen atom in the ring attached to Z and optionally containing a further 1-3 heteroatoms, selected independently from O, S, and N, with the proviso that ring C is not a quinazoline, quinoline or cinnoline group; either any one of G₁, G₂, G₃, G₄ and G₅ is nitrogen and the other four are —CH—; or G₁, G₂, G₃, G₄ and G₅ are all —CH—; Z is —O—, NH—, —S—, CH₂— or a direct substituents R¹ may be attached at any free carbon atom of the indole, azaindole or indazole group; m is an integer from 0 to 2; R^{b} represents hydrogen or another value as defined herein; R¹ represents hydrogen, hydroxy, halogeno, C₁₋₄alkyl, or any other value as defined herein; R² represents hydrogen, hydroxy, halogeno, cyano, nitro, trifluoromethyl, C₁₋₃alkyl, C₁₋₃alkoxy, C₁₋₃alkylsulphanyl, —NR³R⁴ (wherein R³ and R⁴, which may be the same or different, each represents hydrogen or C₁₋₃alkyl), or R⁵X¹— (wherein R⁵ and X¹ are as defined herein) and salts thereof, processes for the preparation of such compounds, pharmaceutical compositions

The present invention relates to indole, azaindole and indazole derivatives, processes for their preparation, pharmaceutical compositions containing them as active ingredient, methods for the treatment of disease states associated with angiogenesis and/or increased vascular permeability, to their use as medicaments and to their use in the manufacture of medicaments for use in the production of antiangiogenic and/or vascular permeability reducing effects in warm-blooded animals such as humans.

Normal angiogenesis plays an important role in a variety of processes including embryonic development, wound healing and several components of female reproductive function. Undesirable or pathological angiogenesis has been associated with disease states including diabetic retinopathy, psoriasis, cancer, rheumatoid arthritis, atheroma, Kaposi's sarcoma and haemangioma (Fan et al, 1995, Trends Pharmacol. Sci. 16: 57-66; Folkman, 1995, Nature Medicine 1: 27-31). Alteration of vascular permeability is thought to play a role in both normal and pathological physiological processes (Cullinan-Bove et al, 1993, Endocrinology 133: 829-837; Senger et al. 1993, Cancer and Metastasis Reviews, 12: 303-324). Several polypeptides with in vitro endothelial cell growth promoting activity have been identified including, acidic and basic fibroblast growth factors (aFGF & bFGF) and vascular endothelial growth factor (VEGF). By virtue of the restricted expression of its receptors, the growth factor activity of VEGF, in contrast to that of the FGFs, is relatively specific towards endothelial cells. Recent evidence indicates that VEGF is an important stimulator of both normal and pathological angiogenesis (Jakeman et al, 1993, Endocrinology, 133: 848-859; Kolch et al, 1995, Breast Cancer Research and Treatment, 36:139-155) and vascular permeability (Connolly et al, 1989, J. Biol. Chem. 264: 20017-20024). Antagonism of VEGF action by sequestration of VEGF with antibody can result in inhibition of tumour growth (Kim et al, 1993, Nature 362: 841-844). Basic FGF (bFGF) is a potent stimulator of angiogenesis (e.g. Hayek et al, 1987, Biochem. Biophys. Res. Commun. 147: 876-880) and raised levels of FGFs have been found in the serum (Fujimoto et al, 1991, Biochem. Biophys. Res. Commun. 180: 386-392) and urine (Nguyen et al, 1993, J. Natl. Cancer. Inst. 85: 241-242) of patients with cancer.

Receptor tyrosine kinases (RTKs) are important in the transmission of biochemical signals across the plasma membrane of cells. These transmembrane molecules characteristically consist of an extracellular ligand-binding domain connected through a segment in the plasma membrane to an intracellular tyrosine kinase domain. Binding of ligand to the receptor results in stimulation of the receptor-associated tyrosine kinase activity which leads to phosphorylation of tyrosine residues on both the receptor and other intracellular molecules. These changes in tyrosine phosphorylation initiate a signalling cascade leading to a variety of cellular responses. To date, at least nineteen distinct RTK subfamilies, defined by amino acid sequence homology, have been identified. One of these subfamilies is presently comprised by the fms-like tyrosine kinase receptor, Flt or Flt1, the kinase insert domain-containing receptor, KDR (also referred to as Flk-1), and another fms-like tyrosine kinase receptor, Flt4. Two of these related RTKs, Flt and KDR, have been shown to bind VEGF with high affinity (De Vries et al, 1992, Science 255: 989-991; Terman et al, 1992, Biochem. Biophys. Res. Comm. 1992, 187: 1579-1586). Binding of VEGF to these receptors expressed in heterologous cells has been associated with changes in the tyrosine phosphorylation status of cellular proteins and calcium fluxes.

The present invention is based on the discovery of compounds that surprisingly inhibit the effects of VEGF, a property of value in the treatment of disease states associated with angiogenesis and/or increased vascular permeability such as cancer, diabetes, psoriasis, rheumatoid arthritis, Kaposi's sarcoma, haemangioma, acute and chronic nephropathies, atheroma, arterial restenosis, autoimmune diseases, acute inflammation, excessive scar formation and adhesions, lymphoedema, endometriosis, dysfunctional uterine bleeding and ocular diseases with retinal vessel proliferation. Compounds of the present invention generally possess higher potency against VEGF receptor tyrosine kinase than against epidermal growth factor (EGF) receptor tyrosine kinase. Compounds of the invention which have been tested possess activity against VEGF receptor tyrosine kinase such that they may be used in an amount sufficient to inhibit VEGF receptor tyrosine kinase whilst demonstrating no significant activity against EGF receptor tyrosine kinase. Compounds of the present invention generally possess higher potency against VEGF receptor tyrosine kinase than against FGF R1 receptor tyrosine kinase. Compounds of the invention which have been tested possess activity against VEGF receptor tyrosine kinase such that they may be used in an amount sufficient to inhibit VEGF receptor tyrosine kinase whilst demonstrating no significant activity against FGF R1 receptor tyrosine kinase.

According to one aspect of the present invention there is provided the use of a compound of the formula I:

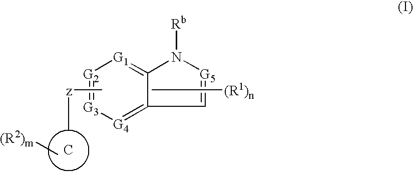

wherein:

ring C is a 9 or 10-membered bicyclic heteroaromatic group containing at least one nitrogen atom in the ring attached to Z and optionally containing a further 1-3 heteroatoms, selected independently from O, S and N, with the proviso that ring C is not a quinazoline, quinoline or cinnoline group;

either any one of G₁, G₂, G₃, G₄ and G₅ is nitrogen and the other four are —CH—, or G₁, G₂, G₃, G₄ and G₅ are all —CH—;

Z is —O—, —NH—, —S—, —CH₂— or a direct bond;

Z is linked to any one of G₁, G₂, G₃ and G₄ which is a free carbon atom;

n is an integer from 0 to 5; any of the substituents R¹ may be attached at any free carbon atom of the indole, azaindole or indazole group, such free carbon atoms may be G₁, G₂, G₃, G₄ or G₅ or may be at the 3-position of the indole, azaindole or indazole group;

m is an integer from 0 to 2;

R^{b} represents hydrogen, C₁₋₄alkyl, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, C₂₋₅alkenylaminoC₁₋₄alkyl, C₂₋₅alkynylaminoC₁₋₄alkyl, —C₁₋₅alkyl(ring A) wherein ring A is selected from azetidinyl, pyrrolidinyl, piperidinyl, piperazinyl, morpholino and thiomorpholino and wherein ring A may bear one or more substituents selected from C₁₋₄alkyl, C₂₋₅alkenyl, C₂₋₅alkynyl, hydroxy, oxo, halogeno, cyano, cyanoC₁₋₄alkyl, C₁₋₄alkylsulphonyl and C₁₋₄alkanoyl;

R¹ represents hydrogen, oxo, hydroxy, halogeno, C₁₋₄alkyl, C₁₋₄alkoxy, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, —C₁₋₅alkyl(ring B) wherein ring B is selected from azetidinyl, pyrrolidiniyl, piperidinyl, piperazinyl, N-methylpiperazinyl, N-ethylpiperazinyl, morpholino and thiomorpholino;

R² represents hydrogen, hydroxy, halogeno, cyano, nitro, trifluoromethyl, C₁₋₃alkyl, C₁₋₃alkoxy, C₁₋₃alkylsulphanyl, —NR³R⁴ (wherein R³ and R⁴, which may be the same or different, each represents hydrogen or C₁₋₃alkyl), or R⁵X¹— (wherein X¹ represents a direct bond, —O—, —CH₂—, —OC(O)—, —C(O)—, —S—, —SO—, —SO₂—, —NR⁶C(O)—, —C(O)NR⁷—, —SO₂NR⁸—, —NR⁹SO₂— or —NR¹⁰— (wherein R⁶, R⁷, R¹, R⁹ and R¹⁰ each independently represents hydrogen, C₁₋₃alkyl or C₁₋₃alkoxyC₂₋₃alkyl), and R⁵ is selected from one of the following twenty-two groups:

1) hydrogen, oxiranylC₁₋₄alkyl or C₁₋₅alkyl which may be unsubstituted or which may be substituted with one or more groups selected from hydroxy, fluoro, chloro, bromo and amino;

2) C₁₋₅alkylX²C(O)R¹¹ (wherein X² represents —O— or —NR¹² (in which R¹² represents hydrogen, C₁₋₃alkyl or C₁₋₃alkoxyC₂₋₃alkyl) and R¹¹ represents C₁₋₃alkyl, —NR¹³R¹⁴ or —OR¹⁵ (wherein R¹³, R¹⁴ and R¹⁵ which maybe the same or different each represents hydrogen, C₁₋₅alkyl or C₁₋₃alkoxyC₂₋₃alkyl));

3) C₁₋₅alkylX³R¹⁶ (wherein X³ represents —O—, —S—, —SO—, —SO₂—, —OC(O)—, —NR¹⁷C(O)—, —C(O)NR¹⁸—, —SO₂NR¹⁹—, —NR²⁰SO₂— or —NR²¹— (wherein R¹⁷, R¹⁸, R¹⁹, R²⁰ and R²¹ each independently represents hydrogen, C₁₋₃alkyl or C₁₋₃alkoxyC₂₋₃alkyl) and R¹⁶ represents hydrogen, C₁₋₃alkyl, cyclopentyl, cyclohexyl or a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonyl C₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

4) C₁₋₅alkylX⁴C₁₋₅alkylX⁵R²² (wherein X⁴ and X⁵ which may be the same or different are each —O—, —S—, —SO—, —SO₂—, —NR²³C(O)—, —C(O)NR²⁴, —SO₂NR²⁵, —NR²⁶SO₂— or NR²⁷— (wherein R²³, R²⁴, R²⁵, R²⁶ and R²⁷ each independently represents hydrogen, C₁₋₃alkyl or C₁₋₃alkoxyC₂₋₃alkyl) and R²² represents hydrogen, C₁₋₃alkyl or C₁₋₃alkoxyC₂₋₃alkyl);

5) R²⁸ (wherein R²⁸ is a 4-, 5- or 6-membered saturated heterocyclic group (linked via carbon or nitrogen) with 1-2 heteroatoms, selected independently from O, S and N, which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkanoyl, C₁₋₄alkoxyC₁₋₄alkyl, C₁₋₄alkylsulphonyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or l, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

6) C₁₋₅alkylR²⁸ (wherein R²⁸ is as defined hereinbefore);

7) C₂₋₅alkenylR²⁸ (wherein R²⁸ is as defined hereinbefore);

8) C₂₋₅alkynylR²⁸ (wherein R²⁸ is as defined hereinbefore);

9) R²⁹ (wherein R²⁹ represents a pyridone group, a phenyl group or a 5-6-membered aromatic heterocyclic group (linked via carbon or nitrogen) with 1-3 heteroatoms selected from O, N and S, which pyridone, phenyl or aromatic heterocyclic group may carry up to 5 substituents selected from hydroxy, halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, —C(O)NR³⁰R³¹, —NR³²C(O)R³³ (wherein R³⁰, R³¹, R³² and R³³, which may be the same or different, each represents hydrogen, C₁₋₄alkyl or C₁₋₃alkoxyC₂₋₃alkyl) and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

10) C₁₋₅alkylR²⁹ (wherein R²⁹ is as defined hereinbefore);

11) C₂₋₅alkenylR²⁹ (wherein R²⁹ is as defined hereinbefore);

12) C₂₋₅alkynylR²⁹ (wherein R²⁹ is as defined hereinbefore);

13) C₁₋₅alkylX⁶R²⁹ (wherein X⁶ represents —O—, —S—, —SO—, —SO₂—, —NR³⁴C(O)—, —C(O)NR^{35—, SO}₂NR³⁶, —NR³⁷SO₂— or —NR³⁸— (wherein R³⁴, R³⁵, R³⁶, R³⁷ and R³⁸ each independently represents hydrogen, C₁₋₃alkyl or C₁₋₃alkoxyC₂₋₃alkyl) and R²⁹ is as defined hereinbefore);

14) C₂₋₅alkenylX⁷R²⁹ (wherein X⁷ represents —O—, —S—, —SO—, —SO₂—, —NR³⁹C(O)—, —C(O)NR⁴⁰—, —SO₂NR⁴¹, —NR⁴²SO₂— or —NR⁴³— (wherein R³⁹, R⁴⁰, R⁴¹, R⁴² and R⁴³ each independently represents hydrogen, C₁₋₃alkyl or C₁₋₃alkoxyC₂₋₃alkyl) and R²⁹ is as defined hereinbefore);

15) C₂₋₅alkynylX⁸R²⁹ (wherein X⁸ represents —O—, —S—, —SO—, —SO₂—, —NR⁴⁴C(O)—, —C(O)NR⁴⁵—, SO₂NR⁴⁶—, —NR⁴⁷SO₂— or —NR⁴⁸— (wherein R⁴⁴, R⁴⁵, R⁴⁶, R⁴⁷ and R⁴⁸ each independently represents hydrogen, C₁₋₃alkyl or C₁₋₃alkoxyC₂₋₃alkyl) and R²⁹ is as defined hereinbefore);

16) C₁₋₄alkylX⁹C₁₋₄alkylR²⁹ (wherein X⁹ represents —O—, —S—, —SO—, —SO₂—, —NR⁴⁹C(O)—, —C(O)NR⁵⁰—, —SO₂NR^{51—, —NR}⁵²SO₂— or —NR⁵³— (wherein R⁴⁹, R⁵⁰, R⁵¹, R⁵² and R⁵³ each independently represents hydrogen, C₁₋₃alkyl or C₁₋₃alkoxyC₂₋₃alkyl) and R²⁹ is as defined hereinbefore);

17) C₁₋₄alkylX⁹C₁₋₄alkylR²⁸ (wherein X⁹ and R²⁸ are as defined hereinbefore);

18) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkynyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

20) C₂₋₅alkenylX⁹C₁₋₄alkylR²⁸ (wherein X⁹ and R²⁸ are as defined hereinbefore);

21) C₂₋₅alkynylX⁹C₁₋₄alkylR²⁸ (wherein X⁹ and R²⁸ are as defined hereinbefore); and

22) C₁₋₄alkylR⁵⁴(C₁₋₄alkyl)_{q}(X⁹)ᵣR⁵ (wherein X⁹ is as defined hereinbefore, q is 0 or 1, r is 0 or 1, and R⁵⁴ and R⁵⁵ are each independently selected from hydrogen, C₁₋₃alkyl, cyclopentyl, cyclohexyl and a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, Cl 4alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl), with the proviso that R⁵⁴ cannot be hydrogen);

and additionally wherein any C₁₋₅alkyl, C₂₋₅alkenyl or C₂₋₅alkynyl group in R⁵X¹— may bear one or more substituents selected from hydroxy, halogeno and amino);

or a salt thereof, or a prodrug thereof for example an ester or an amide, in the manufacture of a medicament for use in the production of an antiangiogenic and/or vascular permeability reducing effect in warm-blooded animals such as humans.

According to another aspect of the present invention there is provided the use of a compound of the formula I¹:

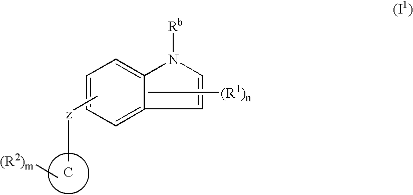

wherein:

ring C is a 9 or 10-membered bicyclic heteroaromatic group containing at least one nitrogen atom in the ring attached to Z and optionally containing a further 1-3 heteroatoms, selected independently from O, S and N, with the proviso that ring C is not a quinazoline, quinoline or cinnoline group;

Z is —O—, —NH—, —S—, —CH₂— or a direct bond; Z is linked to the benz ring of the indole group at any of the positions 4-, 5-, 6- or 7- of the indole group;

n is an integer from 0 to 5; any of the substitutents R¹ maybe attached at any free carbon atom of the indole group, such free carbon atoms maybe at positions 2-, 3-, 4-, 5-, 6-, or 7- of the indole group;

m is an integer from 0 to 2;

R^{b} represents hydrogen, C₁₋₄alkyl, C₁₋₃alkoxyC₁₋₄alkyl, aminoC₁₋₄alkyl, C₁₋₃alkylaminoC₁₋₄alkyl, di(C₁₋₃alkyl)aminoC₁₋₄alkyl, —C₁₋₅alkyl(ring A) wherein ring A is selected from azetidinyl, pyrrolidinyl, piperidinyl, piperazinyl, N-methylpiperazinyl, N-ethylpiperazinyl, morpholino and thiomorpholino;

R¹ represents hydrogen, oxo, hydroxy, halogeno, C₁₋₄alkyl, C₁₋₄alkoxy, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, —C₁₋₅alkyl(ring B) wherein ring B is selected from azetidinyl, pyrrolidinyl, piperidinyl, piperazinyl, N-methylpiperazinyl, N-ethylpiperazinyl, morpholino and thiomorpholino;

R² represents hydrogen, hydroxy, halogeno, cyano, nitro, trifluoromethyl, C-3alkyl, C₁₋₃alkoxy, C₁₋₃alkylsulphanyl, —NR³R⁴ (wherein R³ and R⁴, which may be the same or different, each represents hydrogen or C₁₋₃alkyl), or R⁵X¹— (wherein X¹ represents a direct bond, —O—, —CH₂—, —OC(O)—, —C(O)—, —S—, —SO—, —SO₂—, —NR⁶C(O)—, —C(O)NR⁷—, —SO₂NR⁸—, —NR⁹SO₂— or —NR¹⁰— (wherein R⁶, R⁷, R⁸, R⁹ and R¹⁰ each independently represents hydrogen, C₁₋₃alkyl or C₁₋₃alkoxyC₂₋₃alkyl), and R⁵ is selected from one of the following twenty-two groups:

1) hydrogen, oxiranylC₁₋₄alkyl or C₁₋₅alkyl which may be unsubstituted or which may be substituted with one or more groups selected from hydroxy, fluoro, chloro, bromo and amino;

2) C₁₋₅alkylX²C(O)R¹¹ (wherein X² represents —O— or —NR¹²— (in which R¹² represents hydrogen, C₁₋₃alkyl or C₁₋₃alkoxyC₂₋₃alkyl) and R¹¹ represents C₁₋₃alkyl, —NR¹³R¹⁴ or —OR¹⁵ (wherein R¹³, R¹⁴ and R¹⁵ which may be the same or different each represents hydrogen, C₁₋₅alkyl or C₁₋₃alkoxyC₂₋₃alkyl));

3) C₁₋₅alkylX³R¹⁶ (wherein X³ represents —O—, —S—, —SO—, —SO₂—, —OC(O)—, —NR¹⁷C(O)—, —C(O)NR¹⁸—, SO₂NR¹⁹—, NR²⁰SO₂— or NR²¹— (wherein R⁷, R⁸, R⁹, R²⁰ and R²¹ each independently represents hydrogen, C₁₋₃alkyl or C₁₋₃alkoxyC₂₋₃alkyl) and R¹⁶ represents hydrogen, C₁₋₃alkyl, cyclopentyl, cyclohexyl or a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkylaminoC₁₋₄alkyl, C₁₋₄alkylaminoC₁₋₄alkoxy, di(C₁₋₄alkyl)aminoC₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

4) C₁₋₅alkylX⁴C₁₋₅alkylX⁵R²² (wherein X⁴ and X⁵ which may be the same or different are each —O—, —S—, —SO—, —SO₂—, —NR²³C(O)—, —C(O)NR²⁴—, —SO₂NR²⁵—, NR²⁶SO₂— or —NR²⁷— (wherein R²³, R²⁴, R²⁵, R²⁶ and R²⁷ each independently represents hydrogen, C₁₋₃alkyl or C₁₋₃alkoxyC₂₋₃alkyl) and R²² represents hydrogen, C₁₋₃alkyl or C₁₋₃alkoxyC₂₋₃alkyl);

5) R²⁸ (wherein R²⁸ is a 4-, 5- or 6-membered saturated heterocyclic group (linked via carbon or nitrogen) with 1-2 heteroatoms, selected independently from O, S and N, which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄-alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

6) C₁₋₅alkylR²⁸ (wherein R²⁸ is as defined hereinbefore);

7) C₂₋₅alkenylR²⁸ (wherein R²⁸ is as defined hereinbefore);

8) C₂₋₅alkynylR²⁸ (wherein R²⁸ is as defined hereinbefore);

9) R²⁹ (wherein R²⁹ represents a pyridone group, a phenyl group or a 5-6-membered aromatic heterocyclic group (linked via carbon or nitrogen) with 1-3 heteroatoms selected from O, N and S, which pyridone, phenyl or aromatic heterocyclic group may carry up to 5 substituents selected from hydroxy, halogeno, amino, C₁₋₄alkyl, C₁₋₄alkoxy, C₁₋₄hydroxyalkyl, C₁₋₄aminoalkyl, C₁₋₄alkylamino, C₁₋₄hydroxyalkoxy, carboxy, trifluoromethyl, cyano, —C(O)NR³⁰R³¹, —NR³²C(O)R³³ (wherein R³⁰, R³¹, R³² and R³³, which may be the same or different, each represents hydrogen, C₁₋₄alkyl or C₁₋₃alkoxyC₂₋₃alkyl) and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

10) C₁₋₅alkylR²⁹ (wherein R²⁹ is as defined hereinbefore);

11) C₂₋₅alkenylR²⁹ (wherein R²⁹ is as defined hereinbefore);

12) C₂₋₅alkynylR²⁹ (wherein R²⁹ is as defined hereinbefore);

13) C₁₋₅alkylX⁶R²⁹ (wherein X⁶ represents —O—, —S—, —SO—, —SO₂—, —NR³⁴C(O)—, —C(O)NR³⁵—, —SO₂NR^{36—, —NR}³⁷SO₂— or —NR³⁸— (wherein R³⁴, R³⁵, R³⁶, R³⁷ and R³⁸ each independently represents hydrogen, C₁₋₃alkyl or C₁₋₃alkoxyC₂₋₃alkyl) and R²⁹ is as defined hereinbefore);

14) C₂₋₅alkenylX⁷R²⁹ (wherein X⁷ represents —O—, —S—, —SO—, —SO₂—, —NR³⁹C(O)—, —C(O)NR⁴⁰—, —SO₂NR⁴¹—, —NR⁴²SO₂— or NR⁴³— (wherein R³⁹, R⁴⁰, R⁴¹, R⁴² and R⁴³ each independently represents hydrogen, C₁₋₃alkyl or C₁₋₃alkoxyC₂₋₃alkyl) and R²⁹ is as defined hereinbefore);

15) C₂₋₅alkynylX⁸R²⁹ (wherein X⁸ represents —O—, —S—, —SO—, —SO₂—, —NR⁴⁴C(O)—, —C(O)NR⁴⁵—, —SO₂NR⁴⁶—, NR⁴⁷SO₂— or —NR⁴⁸— (wherein R⁴⁴, R⁴⁵, R⁴⁶, R⁴⁷ and R⁴⁸ each independently represents hydrogen, C₁₋₃alkyl or C₁₋₃alkoxyC₂₋₃alkyl) and R²⁹ is as defined hereinbefore);

16) C₁₋₄alkylX⁹C₁₋₄alkylR²⁹ (wherein X⁹ represents —O—, —S—, —SO—, —SO₂—, —NR⁴⁹C(O)—, —C(O)NR⁵⁰—, —SO₂NR⁵¹—, —NR⁵²SO₂— or —NR⁵³— (wherein R⁴⁹, R⁵⁰, R⁵¹, R⁵² and R⁵³ each independently represents hydrogen, C₁₋₃alkyl or C₁₋₃alkoxyC₂₋₃alkyl) and R²⁹ is as defined hereinbefore);

17) C₁₋₄alkylX⁹C₁₋₄alkylR²⁸ (wherein X⁹ and R²⁸ are as defined hereinbefore);

18) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkynyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

20) C₂₋₅alkenylX⁹C₁₋₄alkylR²⁸ (wherein X⁹ and R²⁸ are as defined hereinbefore);

21) C₂₋₅alkynylX⁹C₁₋₄alkylR²⁸ (wherein X⁹ and R²⁸ are as defined hereinbefore); and

22) C₁₋₄alkylR⁵⁴(C₁₋₄alkyl)_{q}(X⁹)ᵣR⁵⁵ (wherein X⁹ is as defined hereinbefore, q is 0 or 1, r is 0 or 1, and R⁵⁴ and R⁵⁵ are each independently selected from hydrogen, C₁₋₃alkyl, cyclopentyl, cyclohexyl and a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl), with the proviso that R⁵⁴ cannot be hydrogen);

and additionally wherein any C₁₋₅alkyl, C₂₋₅alkenyl or C₂₋₅alkynyl group in R⁵X¹— may bear one or more substituents selected from hydroxy, halogeno and amino);

or a salt thereof, or a prodrug thereof for example an ester or an amide, in the manufacture of a medicament for use in the production of an antiangiogenic and/or vascular permeability reducing effect in warm-blooded animals such as humans.

Preferably ring C is selected from one of the following seven moieties:

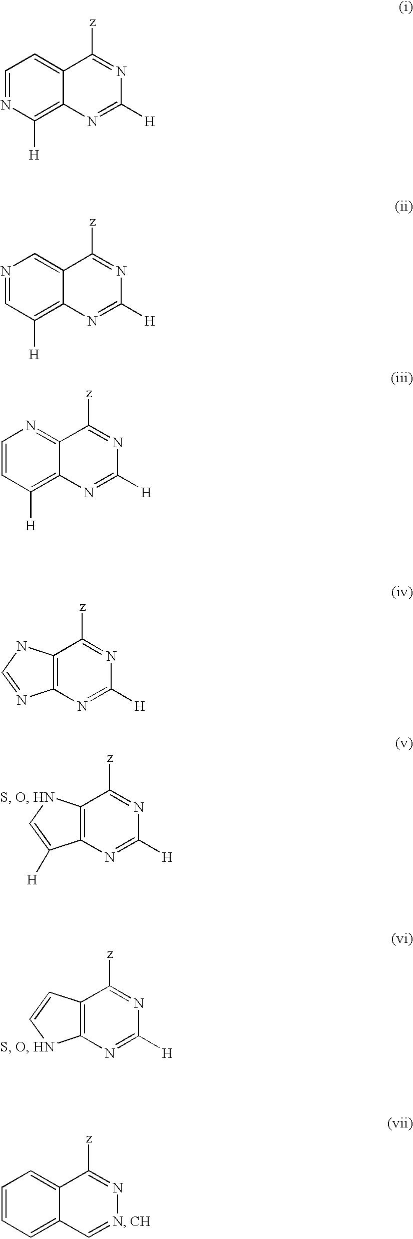

wherein Z is as defined hereinbefore but is not part of ring C, it is shown for the purpose of clarity, and wherein alternatives for the values at certain positions of the rings are indicated by the possible values separated by commas.

More preferably ring C is a thienopyrimidine ring or a phthalazine ring.

Preferably Z is —O—, NH, —S— or a direct bond.

More preferably Z is —O—, —NH— or —S—.

Particularly Z is —O— or —NH—, especially —O—.

Preferably Z is linked to the indole, azaindole or indazole group at the 5- or 6-positions of the indole, azaindole or indazole group.

More preferably Z is linked to the indole, azaindole or indazole group at the 5-position of the indole, azaindole or indazole group.

Preferably Z is linked to an indole group at the 5- or 6-positions of the indole group.

More preferably Z is linked to an indole group at the 5-position of the indole group.

Preferably R^{b} represents hydrogen, C₁₋₂alkyl, C₂₋₃alkenylaminoC₂₋₃alkyl, C₂₋₃alkynylaminoC₂₋₃alkyl or —C₂₋₄alkyl(ring A) wherein ring A is selected from piperidinyl and piperazinyl and wherein ring A may bear one or more substituents selected from C₁₋₂alkyl, C₂₋₃alkenyl, C₂₋₃alkynyl, hydroxy, cyano, cyanoC₁₋₂alkyl, C₁₋₂alkylsulphonyl and C₁₋₂alkanoyl.

More preferably R¹ represents hydrogen, methyl, C₂₋₃alkenylaminoC₂₋₃alkyl, C₂₋₃alkynylaminoC₂₋₃alkyl or —C₂₋₃alkyl(ring A) wherein ring A is selected from 4-acetylpiperazin-1-yl, 4-methylsulphonylpiperazin-1-yl, 4-cyanopiperazin-1-yl, 4-cyanomethylpiperazin-1-yl, 4-(prop-2-en-1-yl)piperazin-1-yl, 4-(prop-2-yn-1-yl)piperazin-1-yl and 4-hydroxypiperidino.

Particularly R^{b} is hydrogen or methyl, especially hydrogen.

Advantageously R¹ represents hydrogen, oxo, hydroxy, halogeno, C₁₋₄alkyl, C₁₋₄alkoxy, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, —C₁₋₅alkyl(ring B) wherein ring B is selected from azetidin-1-yl, pyrrolidin-1-yl, piperidin-1-yl, piperazin-1-yl, N-methylpiperazin-1-yl, N-ethylpiperazin-1-yl, morpholinio and thiomorpholino.

Particularly R¹ represents methyl, ethyl, trifluoromethyl or halogeno.

Especially R¹ represents methyl, fluoro, chloro or bromo, more especially methyl or fluoro.

Preferably n is an integer from 0 to 3.

More preferably n is 0, 1 or 2.

According to one aspect of the present invention GI is nitrogen and G₂, G₃, G₄ and G₅ are —CH— forming an azaindole moiety which may bear one or more substituents R¹ as defined hereinbefore.

According to another aspect of the present invention G₅ is nitrogen and G₁, G₂, G₃ and G₄ are —CH— forming an indazole moiety which may bear one or more substituents R¹ as defined hereinbefore.

According to another aspect of the present invention G₁, G₂, G₃, G₄ and G₅ are all —CH— forming an indole moiety which may bear one or more substituents R¹ as defined hereinbefore.

In one embodiment of the invention the optionally substituted indole, azaindole or indazole moiety of formula II:

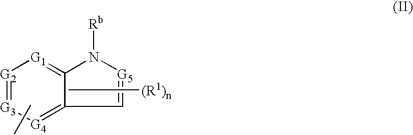

wherein R¹, R^{b}, G₁, G₂, G₃, G₄ and G₅ and n are as defined hereinbefore;

is selected from the indole moieties:

4-fluoro-2-methylindol-5-yl, 2-methylindol-5-yl, 2-methylindol-6-yl, 2,3-dimethylindol-5-yl, 1-methylindol-5-yl, 1,2-dimethylindol-5-yl, 4-fluoroindol-5-yl, 6-fluoroindol-5-yl, indol-S-yl and 3-methylindol-5-yl,

the azaindole moieties:

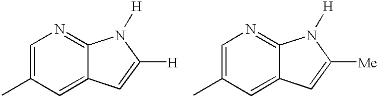

1H-pyrrolo[2,3-b]pyridin-5-yl and 2-methyl-1H-pyrrolo[2,3-b]pyridin-5-yl,

and the indazole moiety:

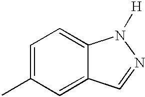

1H-indazol-5-yl.

The indole moieties are preferred over the azaindole and indazole moieties.

In one embodiment of the invention the optionally substituted indole moiety of formula II¹:

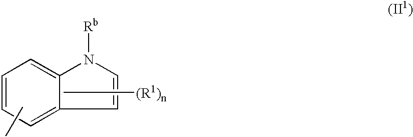

wherein R¹, R^{b} and n are as defined hereinbefore;

is selected from 4-fluoro-2-methylindol-5-yl, 2-methylindol-5-yl, 2-methylindol-6-yl, 2,3-dimethylindol-5-yl, 1-methylindol-5-yl, 1,2-dimethylindol-5-yl, 4-fluoroindol-5-yl, 6-fluoroindol-5-yl and indol-5-yl.

Particularly the optionally substituted indole moiety of formula II is selected from 4-fluoro-2-methylindol-5-yl, 4-fluoroindol-5-yl and 6-fluoroindol-5-yl, more especially from 4-fluoro-2-methylindol-5-yl.

Preferably m is 1 or 2.

Advantageously X¹ represents a direct bond, —O—, —S—, —NR⁶C(O)—, —NR⁹SO₂— or —NR¹⁰— (wherein R⁶, R⁹ and R¹⁰ each independently represents hydrogen, C₁₋₂alkyl or C₁₋₂alkoxyethyl).

Preferably X¹ represents a direct bond, —O—, —S—, —NR⁶C(O)—, —NR⁹SO₂— (wherein R⁶ and R⁹ each independently represents hydrogen or C₁₋₂alkyl) or NH.

More preferably X¹ represents —O—, —S—, —NR⁶C(O)— (wherein R⁶ represents hydrogen or C₁₋₂alkyl) or NH.

Particularly X¹ represents —O— or —NR⁶C(O)— (wherein R⁶ represents hydrogen or C₁₋₂alkyl), more particularly —O— or —NHC(O)—, especially —O—.

According to another aspect of the present invention X¹ represents —O— or a direct bond.

Advantageously X² represents —O— or NR¹² (wherein R¹² represents hydrogen, C₁₋₃alkyl or C₁₋₂alkoxyethyl).

Advantageously X³ represents —O—, —S—, —SO—, —SO₂—, —NR¹⁷C(O)—, —NR²⁰SO₂— or —NR²¹— (wherein R¹⁷, R²⁰ and R²¹ each independently represents hydrogen, C₁₋₂alkyl or C₁₋₂alkoxyethyl).

Preferably X³ represents —O—, —S—, —SO—, —SO₂— or —NR²¹— (wherein R²¹ represents hydrogen, C₁₋₂alkyl or C₁₋₂alkoxyethyl).

More preferably X³ represents —O— or —NR²¹— (wherein R²¹ represents hydrogen or C₁₋₂alkyl).

According to another aspect of the present invention X³ represents —O—, —SO₂—, —NR²⁰SO₂— or —NR²¹— (wherein R²⁰ and R²¹ each independently represents hydrogen, C₁₋₂alkyl or C₁₋₂alkoxyethyl).

Advantageously X⁴ and X⁵ which may be the same or different each represents —O—, —S—, —SO—, —SO₂— or —NR²⁷— (wherein R²⁷ represents hydrogen, C₁₋₃alkyl or C₁₋₂alkoxyethyl).

Preferably X⁴ and X⁵ which may be the same or different each represents —O—, —S— or —NR²⁷— (wherein R²⁷ represents hydrogen, C₁₋₂alkyl or C₁₋₂alkoxyethyl).

More preferably X⁴ and X⁵ which may be the same or different each represents —O— or —NH—.

Especially X⁴ and X⁵ each represents —O—.

Advantageously X⁶ represents —O—, —S— or —NR³⁸— (wherein R³⁸ represents hydrogen, C₁₋₂alkyl or C₁₋₂alkoxyethyl).

Preferably X⁶ represents —O— or —NR³⁸— (wherein R³⁸ represents hydrogen or C₁₋₂alkyl).

Especially X⁶ represents —O—.

Advantageously X⁷ represents —O—, —S— or —NR⁴³— (wherein R⁴³ represents hydrogen, C₁₋₂alkyl or C₁₋₂alkoxyethyl).

Preferably X⁷ represents —O— or —NR⁴³— (wherein R⁴³ represents hydrogen or C₁₋₂alkyl).

Advantageously X⁸ represents —O—, —S— or —NR⁴⁸— (wherein R⁴⁸ represents hydrogen, C₁₋₂alkyl or C₁₋₂alkoxyethyl).

Preferably X⁸ represents —O— or —NR⁴⁸— (wherein R⁴⁸ represents hydrogen or C₁₋₂alkyl).

Advantageously X⁹ represents —O—, —S— or —NR⁵³— (wherein R⁵³ represents hydrogen, C₁₋₂alkyl or C₁₋₂alkoxyethyl).

Preferably X⁹ represents —O— or —NR⁵³— (wherein R⁵³ represents hydrogen or C₁₋₂alkyl).

According to another aspect of the present invention X⁹ represents —O—, —CONR⁵⁰— or —NR⁵³— (wherein R⁵⁰ and R⁵³ each independently represents hydrogen or C₁₋₂alkyl).

Conveniently R²⁸ is pyrrolidinyl, piperazinyl, piperidinyl, imidazolidinyl, azetidinyl, morpholino or thiomorpholino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl).

Advantageously R²⁸ is pyrrolidinyl, piperazinyl, piperidinyl, azetidinyl, morpholino or thiomorpholino which group may bear 1 or 2 substituents selected from oxo, hydroxy, halogeno, cyano, C₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino).

In one embodiment of the present invention R²⁸ is pyrrolidinyl, piperazinyl, piperidinyl, azetidinyl, morpholino or thiomorpholino which group may bear 1 or 2 substituents selected from a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino).

Particularly R²³ is pyrrolidinyl, piperazinyl, piperidinyl, azetidinyl, morpholino or thiomorpholino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl and C₁₋₂alkylsulphonylC₁₋₃alkyl.

According to another aspect of the present invention, preferably R²⁸ is pyrrolidinyl, piperazinyl, piperidinyl, morpholino or thiomorpholino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl and C₁₋₂alkylsulphonylC₁₋₃alkyl.

Where R²⁹ is a 5-6-membered aromatic heterocyclic groups it preferably has 1 or 2 heteroatoms, selected from O, N and S, of which more preferably one is N, and may be substituted as hereinbefore defined.

R²⁹ is particularly a pyridone, phenyl, pyridyl, imidazolyl, thiazolyl, thienyl, triazolyl or pyridazinyl group which group may be substituted as hereinbefore defined, more particularly a pyridone, pyridyl, imidazolyl, thiazolyl or triazolyl group, especially a pyridone, pyridyl, imidazolyl or triazolyl group which group may be substituted as hereinbefore defined.

In one embodiment of the invention R²⁹ represents a pyridone, phenyl or 5-6-membered aromatic heterocyclic group with 1 to 3 heteroatoms selected from O, N and S, which group may preferably carry up to 2 substituents, more preferably up to one substituent, selected from the group of substituents as hereinbefore defined.

In the definition of R²⁹, conveniently substituents are selected from halogeno, C₁₋₄alkyl, C₁₋₄alkoxy, cyano and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl).

In the definition of R²⁹, more conveniently substituents are selected from chloro, fluoro, methyl, ethyl and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino).

According to another emodiment of the present invention in the definition of R²⁹, conveniently substituents are selected from halogeno, C₁₋₄alkyl, C₁₋₄alkoxy and cyano, more conveniently substituents are selected from chloro, fluoro, methyl and ethyl.

Advantageously R⁵⁴ and R⁵⁵ are each independently a 4-, 5- or 6-membered saturated heterocyclic group with 1-2 heteroatoms, selected independently from O, S and N, which cyclic group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl and a group —(—O—)f(C₁₋₃alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₃alkyl).

Preferably R⁵⁴ and R⁵⁵ are each selected from pyrrolidinyl, piperazinyl, piperidinyl, imidazolidinyl, azetidinyl, morpholino and thiomorpholino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl).

More preferably R⁵⁴ and R⁵⁵ are each selected from pyrrolidinyl, piperazinyl, piperidinyl, azetidinyl, morpholino and thiomorpholino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino).

Particularly R⁵⁴ and R⁵⁵ are each selected from pyrrolidinyl, piperazinyl, piperidinyl, azetidinyl, morpholino and thiomorpholino which group may bear 1 or 2 substituents selected from a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino).

More particularly R⁵⁴ and R⁵⁵ are each selected from pyrrolidinyl, piperazinyl, piperidinyl, azetidinyl, morpholino and thiomorpholino which group is unsubstituted.

Conveniently R² represents hydroxy, halogeno, cyano, nitro, trifluoromethyl, C₁₋₃alkyl, amino or R⁵X¹— [wherein X¹ is as hereinbefore defined and R⁵ is selected from one of the following twenty-two groups:

1) oxiranylC₁₋₄alkyl or C₁₋₅alkyl which may be unsubstituted or which may be substituted with one or more groups selected from fluoro, chloro and bromo, or C₂₋₅alkyl which may be unsubstituted or substituted with one or more groups selected from hydroxy and amino;

2) C₂₋₃alkylX²C(O)R¹¹ (wherein X² is as hereinbefore defined and R¹¹ represents C₁₋₃alkyl, —NR¹³R¹⁴ or —OR¹⁵ (wherein R¹³, R¹⁴ and R¹⁵ which may be the same or different are each C₁₋₄alkyl or C₁₋₂alkoxyethyl));

3) C₂₋₄alkylR³R¹⁶ (wherein X³ is as hereinbefore defined and R¹⁶ represents hydrogen, C₁₋₃alkyl, cyclopentyl, cyclohexyl or a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₃alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ring D (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

4) C₂₋₃alkylX⁴C₂₋₃alkylX⁵R²² (wherein X⁴ and X⁵ are as hereinbefore defined and R²² represents hydrogen or C₁₋₃alkyl);

5) R²⁸ (wherein R²⁸ is as defined hereinbefore);

6) C₁₋₅alkylR⁵⁶ (wherein R⁵⁶ is a 4-, 5- or 6-membered saturated heterocyclic group with 1-2 heteroatoms, selected independently from O, S and N, which heterocyclic group is linked to C₁₋₅alkyl through a carbon atom and which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkanoyl, C₁₋₄alkoxyC₁₋₄alkyl, C₁₋₄alkylsulphonyl, C₁₋₄alkylsulphonylC₁₋₄alkyl, C₁₋₄alkoxycarbonyl, C₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl)) or C₂₋₅alkylR⁵⁷ (wherein R⁵⁷ is a 4-, 5- or 6-membered saturated heterocyclic group with 1-2 heteroatoms, of which one is N and the other may be selected independently from O, S and N, which heterocyclic group is linked to C₂₋₅alkyl through a nitrogen atom and which heterocyclic group may bear 1 or 2 substituents selected from oxo, hydroxy, halogeno, cyano, C, 4cyanoalkyl, C₁₋₄alkyl, C₁₋₄hydroxyalkyl, C₁₋₄alkoxy, C₁₋₄alkanoyl, C₁₋₄alkoxyC₁₋₄alkyl, C₁₋₄alkylsulphonyl, C₁₋₄alkylsulphonylC₁₋₄alkyl, C₁₋₄alkoxycarbonyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

7) C₃₋₄alkenylR⁵⁸ (wherein R⁵⁸ represents R⁵⁶ or R⁵⁷ as defined hereinbefore);

8) C₃₋₄alkynylR⁵⁸ (wherein R⁵⁸ represents R⁵⁶ or R⁵⁷ as defined hereinbefore);

9) R²⁹ (wherein R²⁹ is as defined hereinbefore);

10) C₁₋₅alkylR²⁹ (wherein R²⁹ is as defined hereinbefore);

11) C₃₋₅alkenylR²⁹ (wherein R²⁹ is as defined hereinbefore);

12) C₃₋₅alkylR²⁹ (wherein R²⁹ is as defined hereinbefore);

13) C₁₋₅alkynylR²⁹R²⁹ (wherein X⁶ and is R²⁹ are as defined hereinbefore);

14) C₄₋₅alkenylX⁷R²⁹ (wherein X⁷ and is R²⁹ are as defined hereinbefore);

15) C₄₋₅alkylX⁸R²⁹ (wherein X⁸ and R²⁹ are as defined hereinbefore);

16) C₂₋₃alkylX⁹C₁₋₃alkylR²⁹ (wherein X⁹ and R²⁹ are as defined hereinbefore);

17) C₂₋₃alkylX⁹C₁₋₃alkylR²⁸ (wherein X⁹ and R²⁸ are as defined hereinbefore);

18) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

20) C₂₋₅alkenylX⁹C₁₋₃alkylR²⁸ (wherein X⁹ and R²⁸ are as defined hereinbefore);

21) C₂₋₅alkynylX⁹C₁₋₃alkylR²⁸ (wherein X⁹ and R²⁸ are as defined hereinbefore); and

22) C₁₋₃alkylR⁵⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹— may bear one or more substituents selected from hydroxy, halogeno and amino].

Advantageously R² represents hydroxy, halogeno, cyano, nitro, trifluoromethyl, C₁₋₃alkyl, amino or R⁵X¹— [wherein X¹ is as hereinbefore defined and R⁵ is selected from one of the following twenty-two groups:

1) C₁₋₄alkyl which may be unsubstituted or which may be substituted with one or more groups selected from fluoro, chloro and bromo, or C₂₋₅alkyl which may be unsubstituted or substituted with one or more groups selected from hydroxy and amino;

2) C₂₋₃alkylX²C(O)R¹¹ (wherein X² is as hereinbefore defined and R¹¹ represents —NR¹³R¹⁴ or —OR¹⁵ (wherein R¹³, R¹⁴ and R¹⁵ which may be the same or different are each C₁₋₄alkyl or C₁₋₂alkoxyethyl));

3) C₂₋₄alkylX³R¹⁶ (wherein X³ is as hereinbefore defined and R¹⁶ is a group selected from C₁₋₃alkyl, cyclopentyl, cyclohexyl, pyrrolidinyl, piperazinyl, piperidinyl, imidazolidinyl, azetidinyl and tetrahydropyranyl, which C₁₋₃alkyl group may bear 1 or 2 substituents selected from oxo, hydroxy, halogeno and C₁₋₂alkoxy and which cyclopentyl, cyclohexyl, pyrrolidinyl, piperazinyl, piperidinyl, imidazolidinyl, azetidinyl or tetrahydropyranyl group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl));

4) C₂₋₃alkylX⁴C₂₋₃alkylX⁵R²² (wherein X⁴ and X⁵ are as hereinbefore defined and R²² represents hydrogen or C₁₋₃alkyl);

5) R²⁸ (wherein R²⁸ is as defined hereinbefore);

6) C₁₋₄alkylR⁵⁹ (wherein R⁵⁹ is a group selected from pyrrolidinyl, piperazinyl, piperidinyl, imidazolidin-1-yl, azetidinyl, 1,3-dioxolan-2-yl, 1,3-dioxan-2-yl, 1,3-dithiolan-2-yl and 1,3-dithian-2-yl, which group is linked to C₁₋₄alkyl through a carbon atom and which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkylaminoC₁₋₃alkoxy and a group —(—O—)C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl)) or C₂₋₄alkylR⁶⁰ (wherein R⁶⁰ is a group selected from morpholino, thiomorpholino, azetidin-1-yl, pyrrolidin-1-yl, piperazin-1-yl and piperidino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl));

7) C₃₋₄alkenylR⁶¹ (wherein R⁶¹ represents R⁵⁹ or R⁶⁰ as defined hereinbefore);

8) C₃₋₄alkynylR⁶¹ (wherein R⁶¹ represents R⁵⁹ or R⁶⁰ as defined hereinbefore);

9) R²⁹ (wherein R²⁹ is as defined hereinbefore);

10) C₁₋₄alkylR²⁹ (wherein R²⁹ is as defined hereinbefore);

11) 1-R²⁹prop-1-en-3-yl or 1-R²⁹but-2-en-4-yl (wherein R²⁹ is as defined hereinbefore with the proviso that when R⁵ is 1-R²⁹prop-1-en-3-yl, R²⁹ is linked to the alkenyl group via a carbon atom);

12) 1-R²⁹prop-1-yn-3-yl or 1-R²⁹but-2-yn-4-yl (wherein R²⁹ is as defined hereinbefore with the proviso that when R⁵ is 1-R²⁹prop-1-yn-3-yl, R²⁹ is linked to the alkynyl group via a carbon atom);

13) C₁₋₅alkylX⁶R²⁹ (wherein X⁶ and R²⁹ are as defined hereinbefore);

14) 1-(R²⁹X⁷)but-2-en-4-yl (wherein X⁷ and R²⁹ are as defined hereinbefore);

15)1-R²⁹X⁸)but-2-yn-4-yl (wherein X⁸ and R²⁹ are as defined hereinbefore);

16) C₂₋₃alkyl-X⁹C₁₋₃alkylR²⁹ (wherein X⁹ and R²⁹ are as defined hereinbefore);

17) C₂₋₃alkylX⁹C₁₋₃alkylR²⁸ (wherein X⁹ and R²⁸ are as defined hereinbefore);

18) C₂₋₅alkenyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkynyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

20) C₂₋₄alkenylX⁹C₁₋₃alkylR²⁸ (wherein X⁹ and R²⁸ are as defined hereinbefore);

21) C₂₋₄alkynylX⁹C₁₋₃alkylR²⁸ (wherein X⁹ and R²⁸ are as defined hereinbefore); and

22) C₁₋₃alkylR⁵⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹— may bear one or more substituents selected from hydroxy, halogeno and amino].

Preferably R² represents hydroxy, halogeno, nitro, trifluoromethyl, C₁₋₃alkyl, cyano, amino or R⁵X¹— [wherein X¹ is as hereinbefore defined and R⁵ is selected from one of the following twenty groups:

1) C₁₋₃alkyl which may be unsubstituted or which may be substituted with one or more groups selected from fluoro, chloro and bromo, or C₂₋₃alkyl which may be unsubstituted or substituted with one or more groups selected from hydroxy and amino;

2) 2-(3,3-dimethylureido)ethyl, 3-(3,3-dimethylureido)propyl, 2-(3-methylureido)ethyl, 3-(3-methylureido)propyl, 2-ureidoethyl, 3-ureidopropyl, 2-(N,N-dimethylcarbamoyloxy)ethyl, 3-(N,N-dimethylcarbamoyloxy)propyl, 2-(N-methylcarbamoyloxy)ethyl, 3-(N-methylcarbamoyloxy)propyl, 2-(carbamoyloxy)ethyl, 3-(carbamoyloxy)propyl, or 2-(N-methyl-N-(butoxycarbonyl)amino)ethyl;

3) C₁₋₃alkylX³R¹⁶ (wherein X³ is as hereinbefore defined and R¹⁶ is a group selected from C₁₋₃alkyl, cyclopentyl, cyclohexyl, pyrrolidinyl, piperidinyl, piperazinyl, azetidinyl, imidazolidinyl and tetrahydropyranyl which group is linked to X³ through a carbon atom and which C₁₋₃alkyl group may bear 1 or 2 substituents selected from hydroxy, halogeno and C₁₋₂alkoxy and which cyclopentyl, cyclohexyl, pyrrolidinyl, piperidinyl, piperazinyl, azetidinyl, imidazolidinyl or tetrahydropyranyl group may bear one substituent selected from oxo, hydroxy, halogeno, cyano, C₁₋₂cyanoalkyl, C₁₋₂alkyl, C₁₋₂hydroxyalkyl, C₁₋₂alkoxy, C₁₋₂alkoxyC₁₋₃alkyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));

4) C₂₋₃alkylX⁴C₂₋₃alkylX⁵R²² (wherein X⁴ and X⁵ are as hereinbefore defined and R²² represents hydrogen or C₁₋₂alkyl);

5) R²⁸ (wherein R²⁸ is as defined hereinbefore);

6) C₁₋₃alkylR⁵⁹ (wherein R⁵⁹ is a group selected from pyrrolidinyl, piperazinyl, piperidinyl, azetidinyl, imidazolidinyl, 1,3-dioxolan-2-yl, 1,3-dioxan-2-yl, 1,3-dithiolan-2-yl and 1,3-dithian-2-yl, which group is linked to C₁₋₃alkyl through a carbon atom and which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino)) or C₂₋₃alkylR⁶⁰ (wherein R⁶⁰ is a group selected from morpholino, thiomorpholino, azetidin-1-yl, pyrrolidin-1-yl, piperazin-1-yl and piperidino which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));

7) R²⁹ (wherein R²⁹ is as defined hereinbefore);

8) C₁₋₄alkylR²⁹ (wherein R²⁹ is as defined hereinbefore);

9) 1-R²⁹but-2-en-4-yl (wherein R²⁹ is as defined hereinbefore);

10) 1-R²⁹but-2-yn-4-yl (wherein R²⁹ is as defined hereinbefore);

11) C₁₋₃alkylX⁶R²⁹ (wherein X⁶ and R²⁹ are as defined hereinbefore);

12) 1-(R²⁹X⁷)but-2-en-4-yl (wherein X⁷ and R²⁹ are as defined hereinbefore);

13) 1-(R²⁹X⁸)but-2-yn-4-yl (wherein X⁸ and R²⁹ are as defined hereinbefore);

14) C₂₋₃alkylX⁹C₁₋₃alkylR²⁹ (wherein X⁹ and R²⁹ are as defined hereinbefore);

15) C₂₋₃alkylX⁹C₁₋₃alkylR²⁸ (wherein X⁹ and R²⁸ are as defined hereinbefore);

16) C₂₋₅alkenyl which may be unsubstituted or which may be substituted with one or more fluorine atoms or with one or two groups selected from hydroxy, fluoro, amino, C₁₋₄alkylamino, N N-di(C₁₋₄alkyl) amino, aminosulphonyl, N-C₁₋₄alkylaminosulphonyl and N,N-di(C₁₋₄alkyl)aminosulphonyl;

17) C₂₋₅alkyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

18) C₂₋₃alkenylX⁹C₁₋₃alkylR²⁸ (wherein X⁹ and R²⁸ are as defined hereinbefore);

19) C₂₋₃alkylX⁹C₁₋₃alkylR²⁸ (wherein X⁹ and R²⁸ are as defined hereinbefore); and

20) C₁₋₃alkylR⁵⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹— may bear one or more substituents selected from hydroxy, halogeno and amino].

More preferably R² represents hydroxy, C₁₋₃alkyl, amino or R⁵X¹— [wherein X¹ is as hereinbefore defined and R⁵ represents methyl, ethyl, benzyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl)ethyl, 2-(ethylsulphonyl)ethyl, 2-(N,N-dimethylsulphamoyl)ethyl, 2-N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-(N,N-dimethylamino)propyl, 2-(N,N-diethylamino)ethyl, 3-(N,N-diethylamino)propyl, 2-(N-methyl-N-methylsulphonylamino)ethyl, 3-(N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperdin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-(piperidin-2-yl)propyl, (1-methylpiperidin-3-yl)methyl, (1-methylpiperidin-4-yl)methyl, 2-(4-hydroxypiperidino)ethyl, 3-(4-hydroxypiperidino)propyl, (1-cyanomethylpiperidin-3-yl)methyl, (1-cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-ethoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-4-yl)ethyl, 3-(1-isopropylpiperidin-2-yl)propyl, 3-(1-isopropylpiperidin-3-yl)propyl, 3-(1-isopropylpiperidin-4-yl)propyl, 2-(piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-(piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H-pyrrolidin-5-yl)methyl, (1,3-dioxolan-2-yl)methyl, 2-(1,3-dioxolan-2-yl)ethyl, 2-(2-methoxyethylamino)ethyl, 2-(N-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxethylamino)propyl, 2-methylthiazol-4-ylmethyl, 2-acetamidothiazol-4-ylmethyl, 1-methylimidazol-2-ylmethyl, 2-(imidazol-1-yl)ethyl, 2-(2-methylimidazol-1-yl)ethyl, 2-(2-ethylimidazol-1-yl)ethyl, 3-(2-methylimidazol-1-yl)propyl, 3-(2-ethylimidazol-1-yl)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 3-pyridylmethyl, 2-(3-pyridyl)ethyl, 3-(3-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 2-(4-cyanomethylpiperazin-1-yl)ethyl, 3-(4-cyanomethylpiperazin-1-yl)propyl, 2-(4-acetylpiperazin-1-yl)ethyl, 3-(4-acetylpiperazin-1-yl)propyl, 2-(4-methylsulphonylpiperazin-1-yl)ethyl, 3-(4-methylsulphonylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(1-methylimidazol-4-ylsulphonyl)-N-methyl)amino)ethyl, 2-((N-(3-morpholinopropylsulphonyl)-N-methyl)amino)ethyl, 2-(N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-ylmethyl, 1-(2-azetidinylethyl)piperidin-4-ylmethyl, 1-(3-azetidinylpropyl)piperidin-4-ylmethyl, 2-(1-(2-pyrrolidinylethyl)piperidin-4-yl)ethyl, 2-(1-(3-pyrrolidinylpropyl)piperidin-4-yl)ethyl, 2-(1-(2-piperidinylethyl)piperidin-4-yl)ethyl, 2-(1-(3-piperidinylpropyl)piperidin-4-yl)ethyl, 2-(1-(2-morpholinoethyl)piperidin-4-yl)ethyl, 2-(1-(3-morpholinopropyl)piperidin-4-yl)ethyl, 2-(1-(2-thiomorpholinoethyl)piperidin-4-yl)ethyl, 2-(1-(3-thiomorpholinopropyl)piperidin-4-yl)ethyl, 2-(1-(2-azetidinylethyl)piperidin-4-yl)ethyl, 2-(1-(3-azetidinylpropyl)piperidin-4-yl)ethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2S)-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(1-methylpiperazin-4-yl)-2-hydroxypropyl, (2R)-3-(1-methylpiperazin-4-yl)-2-hydroxypropyl, (2S)-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N,N-diethylamino)-2-hydroxypropyl, 3-(isopropylamino)-2-hydroxypropyl, (2R)-3-(isopropylamino)-2-hydroxypropyl, (2S)-3-(isopropylamino)-2-hydroxypropyl, 3-(N,N-diisopropylamino)-2-hydroxypropyl, (2R)-3-(N,N-diisopropylamino)-2-hydroxypropyl or (2S)-3-(N,N-diisopropylamino)-2-hydroxypropyl].

Particularly R² represents C₁₋₃alkyl, amino or R⁵X¹— [wherein X¹ is as hereinbefore defined and R⁵ represents ethyl, benzyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl)ethyl, 2-(ethylsulphonyl)ethyl, 2-(N,N-dimethylsulphamoyl)ethyl, 2-(N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-(N,N-dimethylamino)propyl, 2-(N,N-diethylamino)ethyl, 3-(N,N-diethylamino)propyl, 2-(N-methyl-N-methylsulphonylamino)ethyl, 3-(N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperidin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-(piperidin-2-yl)propyl, (1-methylpiperidin-3-yl)methyl, (1-methylpiperidin-4-yl)methyl, 2-(4-hydroxypiperidino)ethyl, 3-(4-hydroxypiperidino)propyl, (1-cyanomethylpiperidin-3-yl)methyl, (1cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-methoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-4-yl)ethyl, 3-(1-isopropylpiperidin-2-yl)propyl, 3-(1-isopropylpiperidin-3-yl)propyl, 3-(1-isopropylpiperidin-4-yl)propyl, 2-(piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-(piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H-pyrrolidin-5-yl)methyl, (1,3-dioxolan-2-yl)methyl, 2-(1,3-dioxolan-2-yl)ethyl, 2-(2-methoxyethylamino)ethyl, 2-(X-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxyethylamino)propyl, 2-methylthiazol-4-ylmethyl, 2-acetamidothiazol-4-ylmethyl, 1-methylimidazol-2-ylmethyl, 2-(imidazol-1-yl)ethyl, 2-(2-methylimidazol-1-yl)ethyl, 2-(2-ethylimidazol-1-yl)ethyl, 3-(2-methylimidazol-1-yl)propyl, 3-(2-ethylimidazol-1-yl)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 3-pyridylmethyl, 2-(3-pyridyl)ethyl, 3-(3-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 2-(4-cyanomethylpiperazin-1-yl)ethyl, 3-(4-cyanomethylpiperazin-1-yl)propyl, 2-(4-acetylpiperazin-1-yl)ethyl, 3-(4-acetylpiperazin-1-yl)propyl, 2-(4-methylsulphonylpiperazin-1-yl)ethyl, 3-(4-methylsulphonylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(1-methylimidazol-4-ylsulphonyl)-N-methyl)amino)ethyl, 2-((N-(3-morpholinopropylsulphonyl)-N-methyl)amino)ethyl, 2-((N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-ylmethyl, 1-(2-azetidinylethyl)piperidin-4-ylmethyl, 1-(3-azetidinylpropyl)piperidin-4-ylmethyl, 2-(1-(2-pyrrolidinylethyl)piperidin-4-yl)ethyl, 2-(1-(3-pyrrolidinylpropyl)piperidin-4-yl)ethyl, 2-(1-(2-piperidinylethyl)piperidin-4-yl)ethyl, 2-(1-(3-piperidinylpropyl)piperidin-4-yl)ethyl, 2-(1-(2-morpholinoethyl)piperidin-4-yl)ethyl, 2-(1-(3-morpholinopropyl)piperidin-4-yl)ethyl, 2-(1-(2-thiomorpholinoethyl)piperidin-4-yl)ethyl, 2-(1-(3-thiomorpholinopropyl)piperidin-4-yl)ethyl, 2-(1-(2-azetidinylethyl)piperidin-4-yl)ethyl, 2-(1-(3-azetidinylpropyl)piperidin-4-yl)ethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2S)-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(i-methylpiperazin-4-yl)-2-hydroxypropyl, (2R)-3-(1-methylpiperazin-4-yl)-2-hydroxypropyl, (2S)-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N,N-diethylamino)-2-hydroxypropyl, 3-(isopropylamino)-2-hydroxypropyl, (2R)-3-(isopropylamino)-2-hydroxypropyl, (2S)-3-(isopropylamino)-2-hydroxypropyl, 3-(N,N-diisopropylamino)-2-hydroxypropyl, (2R)-3-(N,N-diisopropylamino)-2-hydroxypropyl or (2S)-3-(N,N-diisopropylamino)-2-hydroxypropyl].

More particularly R² represents C₁₋₃alkyl, amino or R⁵X¹— [wherein X¹ is as hereinbefore defined and R⁵ represents ethyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl) ethyl, 2-(ethylsulphonyl)ethyl, 2-(N,N-dimethylsulphamoyl)ethyl, 2-(N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-(N,N-dimethylamino)propyl, 2-(N,N-diethylamino)ethyl, 3-(N,N-diethylamino)propyl, 2-(N-methyl-N-methylsulphonylamino)ethyl, 3-(N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperidin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-(piperidin-2-yl)propyl, (1-methylpiperidin-3-yl)methyl, (1-methylpiperidin-4-yl)methyl, 2-(4-hydroxypiperidino)ethyl, 3-(4-hydroxypiperidino)propyl, (1-cyanomethylpiperidin-3-yl)methyl, (1-cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-methoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-1-4-yl)ethyl, 3-(1-isopropylpiperidin-2-yl)propyl, 3-(1-isopropylpiperidin-3-yl)propyl, 3-(1-isopropylpiperidin-4-yl)propyl, 2-piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-(piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H[-pyrrolidin-5-yl)methyl, (1,3-dioxolan-2-yl)methyl, 2-(1,3-dioxolan-2-yl)ethyl, 2-(2-methoxyethylamino)ethyl, 2-(N-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxyethylamino)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 3-pyridylmethyl, 2-(3-pyridyl)ethyl, 3-(3-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 2-(4-cyanomethylpiperazin-1-yl)ethyl, 3-(4-cyanomethylpiperazin-1-yl)propyl, 2-(4-acetylpiperazin-1-yl)ethyl, 3-(4-acetylpiperazin-1-yl)propyl, 2-(4-methylsulphonylpiperazin-1-yl)ethyl, 3-(4-methylsulphonylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(3-morpholinopropylsulphonyl)-N-methyl)amino)ethyl, 2-((N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-yl-ethyl, 1-(2-azetidinylethyl)piperidin-4-ylmethyl, 1-(3-azetidinylpropyl)piperidin-4-ylmethyl, 2-(1-(2-pyrrolidinylethyl)piperidin-4-yl)ethyl, 2-(1-(3-pyrrolidinylpropyl)piperidin-4-yl)ethyl, 2-(1-(2-piperidinylethyl)piperidin-4-yl)ethyl, 2-(1-(3-piperidinylpropyl)piperidin-4-yl)ethyl, 2-(1-(2-morpholinoethyl)piperidin-4-yl)ethyl, 2-(1-(3-morpholinopropyl)piperidin-4-yl)ethyl, 2-(1-(2-thiomorpholinoethyl)piperidin-4-yl)ethyl, 2-(1-(3-thiomorpholinopropyl)piperidin-4-yl)ethyl, 2-(1-(2-azetidinylethyl)piperidin-4-yl)ethyl, 2-(1-(3-azetidinylpropyl)piperidin-4-yl)ethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2S-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(1-methylpiperazin-4-yl)-2-hydroxypropyl, (2R)-3-(1-methylpiperazin-4-yl)-2-hydroxypropyl, (2S)-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N,N-diethylamino)-2-hydroxypropyl, 3-(isopropylamino)-2-hydroxypropyl, (2R)-3-(isopropylamino)-2-hydroxypropyl, (2s)—3-(isopropylamino)-2-hydroxypropyl, 3-(N,N-diisopropylamino)-2-hydroxypropyl, (2R)-3-(N,N-diisopropylamino)-2-hydroxypropyl or (2S)-3-(N,N-diisopropylamino)-2-hydroxypropyl].

In another aspect R² represents ethoxy, trifluoromethoxy, 2,2,2-trifluoroethoxy, 2-hydroxyethoxy, 3-hydroxypropoxy, 2-methoxyethoxy, 3-methoxypropoxy, 2-(methylsulphinyl)ethoxy, 2-(methylsulphonyl)ethoxy, 2-(ethylsulphinyl)ethoxy, 2-(ethylsulphonyl)ethoxy, 2-(N,N-dimethylsulphamoyl)ethoxy, 2-(N-methylsulphamoyl)ethoxy, 2-sulphamoylethoxy, 2-(methylamino)ethoxy, 3-(methylamino)propoxy, 2-(ethylamino)ethoxy, 3-(ethylamino)propoxy, 2-(N,N-dimethylamino)ethoxy, 3-(N,N-dimethylamino)propoxy, 2-(N,N-diethylamino)ethoxy, 3-(N,N-diethylamino)propoxy, 2-(N-methyl-N-methylsulphonylamino)ethoxy, 3-(N-methyl-N-methylsulphonylamino)propoxy, 2-morpholinoethoxy, 3-morpholinopropoxy, 2-piperidinoethoxy, 3-piperidinopropoxy, 2-(methylpiperidino)ethoxy, 3-(methylpiperidino)propoxy, 2-(ethylpiperidino)ethoxy, 3-(ethylpiperidino)propoxy, 2-((2-methoxyethyl)piperidino)ethoxy, 3-((2-methoxyethyl)piperidino)propoxy, 2-((2-methylsulphonyl)ethylpiperidino)ethoxy, 3-((2-methylsulphonyl)ethylpiperidino)propoxy, piperidin-3-ylmethoxy, piperidin-4-ylmethoxy, 2-(piperidin-3-yl)ethoxy, 2-(piperidin-4-yl)ethoxy, 3-(piperidin-3-yl)propoxy, 3-(piperidin-4-yl)propoxy, 2-(piperidin-2-yl)ethoxy, 3-(piperidin-2-yl)propoxy, (1-methylpiperidin-3-yl)methoxy, (1-methylpiperidin-4-yl)methoxy, 2-(4-hydroxypiperidino)ethoxy, 3-(4-hydroxypiperidino)propoxy, (1-cyanomethylpiperidin-3-yl)methoxy, (1-cyanomethylpiperidin-4-yl)methoxy, 2-(methylpiperidin-3-yl)ethoxy, 2-(methylpiperidin-4-yl)ethoxy, 2-(1-cyanomethylpiperidin-3-yl)ethoxy, 2-(1-cyanomethylpiperidin-4-yl)ethoxy, 3-(methylpiperidin-3-yl)propoxy, 3-(methylpiperidin-4-yl)propoxy, 3-(1-cyanomethylpiperidin-3-yl)propoxy, 3-(1-cyanomethylpiperidin-4-yl)propoxy, 2-(ethylpiperidin-3-yl)ethoxy, 2-(ethylpiperidin-4-yl)ethoxy, 3-(ethylpiperidin-3-yl)propoxy, 3-(ethylpiperidin-4-yl)propoxy, ((2-methoxyethyl)piperidin-3-yl)methoxy, ((2-methoxyethyl)piperidin-4-yl)methoxy, 2-((2-methoxyethyl)piperidin-3-yl)ethoxy, 2-((2-methoxyethyl)piperidin-4-yl)ethoxy, 3-((2-methoxyethyl)piperidin-3-yl)propoxy, 3-((2-methoxyethyl)piperidin-4-yl)propoxy, (1-(2-methylsulphonylethyl)piperidin-3-yl)methoxy, (1-(2-methylsulphonylethyl)piperidin-4-yl)methoxy, 2-((2-methylsulphonylethyl)piperidin-3-yl)ethoxy, 2-((2-methylsulphonylethyl)piperidin-4-yl)ethoxy, 3-((2-methylsulphonylethyl)piperidin-3-yl)propoxy, 3-((2-methylsulphonylethyl)piperidin-4-yl)propoxy, 1-isopropylpiperidin-2-ylmethoxy, 1-isopropylpiperidin-3-ylmethoxy, 1-isopropylpiperidin-4-ylmethoxy, 2-(1-isopropylpiperidin-2-yl)ethoxy, 2-(1-isopropylpiperidin-3-yl)ethoxy, 2-(1-isopropylpiperidin-4-yl)ethoxy, 3-(1-isopropylpiperidin-2-yl)propoxy, 3-(1-isopropylpiperidin-3-yl)propoxy, 3-(1-isopropylpiperidin-4-yl)propoxy, 2-(piperidin-4-yloxy)ethoxy, 3-(piperidin-4-yloxy)propoxy, 2-(1-(cyanomethyl)piperidin-4-yloxy)ethoxy, 3-(1-(cyanomethyl)piperidin-4-yloxy)propoxy, 2-(1-(2-cyanoethyl)piperidin-4-yloxy)ethoxy, 3-(1-(2-cyanoethyl)piperidin-4-yloxy)propoxy, 2-(piperazin-1-yl)ethoxy, 3-(piperazin-1-yl)propoxy, (pyrrolidin-2-yl)methoxy, 2-(pyrrolidin-1-yl)ethoxy, 3-(pyrrolidin-1-yl)propoxy, (2-oxo-tetrahydro-2H-pyrrolidin-5-yl)methoxy, 5(R)-(2-oxo-tetrahydro-2H-pyrrolidin-5-yl)methoxy, (5S)-(2-oxo-tetrahydro-2H-pyrrolidin-5-yl)methoxy, (1,3-dioxolan-2-yl)methoxy, 2-(1,3-dioxolan-2-yl)ethoxy, 2-(2-methoxyethylamino)ethoxy, 2-(N-(2-methoxyethyl)-N-methylamino)ethoxy, 2-(2-hydroxyethylamino)ethoxy, 3-(2-methoxyethylamino)propoxy, 3-(N-(2-methoxyethyl)-N-methylamino)propoxy, 3-(2-hydroxyethylamino)propoxy, 2-(1,2,3-triazol-1-yl)ethoxy, 2-(1,2,3-triazol-2-yl)ethoxy, 2-(1,2,4-triazol-1-yl)ethoxy, 2-(1,2,4-triazol-4-yl)ethoxy, 4-pyridylmethyl, 2-(4-pyridyl)ethyl, 4-pyridylmethoxy, 2-(4-pyridyl)ethoxy, 3-(4-pyridyl)propoxy, 3-pyridylmethoxy, 2-(3-pyridyl)ethoxy, 3-(3-pyridyl)propoxy, 2-(4-pyridyloxy)ethoxy, 2-(4-pyridylamino)ethoxy, 2-(4-oxo-1,4-dihydro-1-pyridyl)ethoxy, 2-(2-oxo-imidazolidin-1-yl)ethoxy, 3-(2-oxo-imidazolidin-1-yl)propoxy, 2-thiomorpholinoethoxy, 3-thiomorpholinopropoxy, 2-(1,1-dioxothiomorpholino)ethoxy, 3-(1,1-dioxothiomorpholino)propoxy, 2-(2-methoxyethoxy)ethoxy, 2-(4-methylpiperazin-1-yl)ethoxy, 3-(4-methylpiperazin-1-yl)propoxy, 2-(4-cyanomethylpiperazin-1-yl)ethoxy, 3-(4-cyanomethylpiperazin-1-yl)propoxy, 2-(4-acetylpiperazin-1-yl)ethoxy, 3-(4-acetylpiperazin-1-yl)propoxy, 2-(4-methylsulphonylpiperazin-1-yl)ethoxy, 3-(4-methylsulphonylpiperazin-1-yl)propoxy, 3-(methylsulphinyl)propoxy, 3-(methylsulphonyl)propoxy, 3-(ethylsulphinyl)propoxy, 3-(ethylsulphonyl)propoxy, 2-(5-methyl-1,2,4-triazol-1-yl)ethoxy, 2-((N-(3-morpholinopropylsulphonyl)-N-methyl)amino)ethoxy, 2-((N-methyl-N-4-pyridyl)amino)ethoxy, 3-(4-oxidomorpholino)propoxy, 2-(2-(4-methylpiperazin-1-yl)ethoxy)ethoxy, 3-(2-(4-methylpiperazin-1-yl)ethoxy)propoxy, 2-(2-morpholinoethoxy)ethoxy, 3-(2-morpholinoethoxy)propoxy, 2-(tetrahydropyran-4-yloxy)ethoxy, 3-(tetrahydropyran-4-yloxy)propoxy, 2-((2-(pyrrolidin-1-yl)ethyl)carbamoyl)vinyl, 3-((2-(pyrrolidin-1-yl)ethyl)carbamoyl)prop-2-en-1-yloxy, 1-(2-pyrrolidinylethyl)piperidin-4-ylmethoxy, 1-(3-pyrrolidinylpropyl)piperidin-4-ylmethoxy, 1-(2-piperidinylethyl)piperidin-4-ylmethoxy, 1-(3-piperidinylpropyl)piperidin-4-ylmethoxy, 1-(2-morpholinoethyl)piperidin-4-ylmethoxy, 1-(3-morpholinopropyl)piperidin-4-ylmethoxy, 1-(2-thiomorpholinoethyl)piperidin-4-ylmethoxy, 1-(3-thiomorpholinopropyl)piperidin-4-ylmethoxy, 1-(2-azetidinylethyl)piperidin-4-ylmethoxy, 1-(3-azetidinylpropyl)piperidin-4-ylmethoxy, 2-(1-(2-pyrrolidinylethyl)piperidin-4-yl)ethoxy, 2-(1-(3-pyrrolidinylpropyl)piperidin-4-yl)ethoxy, 2-(1-(2-piperidinylethyl)piperidin-4-yl)ethoxy, 2-(1-(3-piperidinylpropyl)piperidin-4-yl)ethoxy, 2-(i-(2-morpholinoethyl)piperidin-4-yl)ethoxy, 2-(1-(3-morpholinopropyl)piperidin-4-yl)ethoxy, 2-(1-(2-thiomorpholinoethyl)piperidin-4-yl)ethoxy, 2-(1-(3-thiomorpholinopropyl)piperidin-4-yl)ethoxy, 2-(1-(2-azetidinylethyl)piperidin-4-yl)ethoxy, 2-(1-(3-azetidinylpropyl)piperidin-4-yl)ethoxy, 3-morpholino-2-hydroxypropoxy, (2R)-3-morpholino-2-hydroxypropoxy, (2S-3-morpholino-2-hydroxypropoxy, 3-piperidino-2-hydroxypropoxy, (2R)-3-piperidino-2-hydroxypropoxy, (2S)-3-piperidino-2-hydroxypropoxy, 3-pyrrolidin-1-yl-2-hydroxypropoxy, (2R)-3-pyrrolidin-1-yl-2-hydroxypropoxy, (2S)-3-pyrrolidin-1-yl-2-hydroxypropoxy, 3-(1-methylpiperazin-4-yl)-2-hydroxypropoxy, (2R)-3-(1-methylpiperazin-4-yl)-2-hydroxypropoxy, (2S)-3-(1-methylpiperazin-4-yl)-2-hydroxypropoxy, 3-(N,N-diethylamino)-2-hydroxypropoxy, (2R)-3-(N,N-diethylamino)-2-hydroxypropoxy, (2S)-3-(N,N-diethylamino)-2-hydroxypropoxy, 3-(isopropylamino)-2-hydroxypropoxy, (2R)-3-(isopropylamino)-2-hydroxypropoxy, (2S)-3-(isopropylamino)-2-hydroxypropoxy, 3-(N,N-diisopropylamino)-2-hydroxypropoxy, (2R)-3-(N,N-diisopropylamino)-2-hydroxypropoxy or (2S)-3-(N,N-diisopropylamino)-2-hydroxypropoxy.

According to another aspect of the present invention conveniently R² represents hydroxy, halogeno, cyano, nitro, trifluoromethyl, C₁₋₃alkyl, amino or R⁵X¹′ [wherein X¹ is as hereinbefore defined and R⁵ is selected from one of the following twenty-two groups:

1) oxiranylC₁₋₄alkyl or C₁₋₅alkyl which may be unsubstituted or which may be substituted with one or more groups selected from fluoro, chloro and bromo, or C₂₋₅alkyl which may be unsubstituted or substituted with one or more groups selected from hydroxy and amino;

2) C₂₋₃alkylX²C(O)R¹¹ (wherein X² is as hereinbefore defined and R¹¹ represents C₁₋₃alkyl, —NR¹³R¹⁴ or —OR¹⁵ (wherein R¹³, R¹⁴ and R¹⁵ which may be the same or different are each C₁₋₄alkyl or C₁₋₂alkoxyethyl));

3) C₂₋₄alkylX³R¹⁶ (wherein X³ is as hereinbefore defined and R¹⁶ represents hydrogen, C₁₋₃alkyl, cyclopentyl, cyclohexyl or a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₃alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

4) C₂₋₃alkylX⁴C₂₋₃alkylX⁵R²² (wherein X⁴ and X⁵ are as hereinbefore defined and R²² represents hydrogen or C₁₋₃alkyl);

5) R²⁸ (wherein R²⁸ is as defined hereinbefore);

6) C₁₋₅alkylR⁵⁶ (wherein R⁵⁶ is a 4-, 5- or 6-membered saturated heterocyclic group with 1-2 heteroatoms, selected independently from O, S and N, which heterocyclic group is linked to C₁₋₅alkyl through a carbon atom and which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl)) or C₂₋₅alkylR⁵⁷ (wherein R⁵⁷ is a 4-, 5- or 6-membered saturated heterocyclic group with 1-2 heteroatoms, of which one is N and the other may be selected independently from O, S and N, which heterocyclic group is linked to C₂₋₅alkyl through a nitrogen atom and which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₄alkyl, C₁₋₄alkoxycarbonyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));

7) C₃₋₄alkenylR⁵⁸ (wherein R⁵⁸ represents R⁵⁶ or R⁵⁷ as defined hereinbefore);

8) C₃₋₄alkynylR⁵⁸ (wherein R⁵⁸ represents R⁵⁶ or R⁵⁷ as defined hereinbefore);

9) R²⁹ (wherein R²⁹ is as defined hereinbefore);

10) C₁₋₅alkylR²⁹ (wherein R²⁹ is as defined hereinbefore);

11) C₃₋₅alkenylR²⁹ (wherein R²⁹ is as defined hereinbefore);

12) C₃₋₅alkynylR²⁹ (wherein R²⁹ is as defined hereinbefore);

13) C₁₋₅alkylX⁶R²⁹ (wherein X⁶ and R²⁹ are as defined hereinbefore);

14) C₄₋₅alkenylR⁷R²⁹ (wherein X⁷ and R²⁹ are as defined hereinbefore);

15) C₄₋₅alkynylX⁸R²⁹ (wherein X⁸ and R²⁹ are as defined hereinbefore);

16) C₂₋₃alkylX⁹C₁₋₃alkylR²⁹ (wherein X⁹ and R²⁹ are as defined hereinbefore);

17) C₂₋₃alkylX⁹C₁₋₃alkylR²⁸ (wherein X⁹ and R²⁸ are as defined hereinbefore);

18) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkynyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

20) C₂₋₅alkenylX⁹C₁₋₃alkylR²⁸ (wherein X⁹ and R²⁸ are as defined hereinbefore);

21) C₂₋₅alkynylX⁹C₁₋₃alkylR²⁸ (wherein X⁹ and R²⁸ are as defined hereinbefore); and

22) C₁₋₃alkylR⁵⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹— may bear one or more substituents selected from hydroxy, halogeno and amino].

According to another aspect of the present invention advantageously R² represents hydroxy, halogeno, cyano, nitro, trifluoromethyl, C₁₋₃alkyl, amino or R⁵X¹′ [wherein X¹ is as hereinbefore defined and R⁵ is selected from one of the following twenty-two groups:

1) C₁₋₄alkyl which may be unsubstituted or which may be substituted with one or more groups selected from fluoro, chloro and bromo, or C₂₋₅alkyl which may be unsubstituted or substituted with one or more groups selected from hydroxy and amino;

2) C₂₋₃alkylX²C(O)R¹¹ (wherein X² is as hereinbefore defined and R¹¹ represents —NR¹³R¹⁴ or —OR¹⁵ (wherein R¹³, R¹⁴ and R¹⁵ which may be the same or different are each C₁₋₄alkyl or C₁₋₂alkoxyethyl));

3) C₂₋₄alkylX³R¹⁶ (wherein X³ is as hereinbefore defined and R¹⁶ is a group selected from C₁₋₃alkyl, cyclopentyl, cyclohexyl, pyrrolidinyl, piperazinyl, piperidinyl, imidazolidinyl, azetidinyl and tetrahydropyranyl, which C₁₋₃alkyl group may bear 1 or 2 substituents selected from oxo, hydroxy, halogeno and C₁₋₂alkoxy and which cyclopentyl, cyclohexyl, pyrrolidinyl, piperazinyl, piperidinyl, imidazolidinyl, azetidinyl or tetrahydropyranyl group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl));

4) C₂₋₃alkylX⁴C₂₋₃alkylX⁵R²² (wherein X⁴ and X⁵ are as hereinbefore defined and R²² represents hydrogen or C₁₋₃alkyl);

5) R²⁸ (wherein R²⁸ is as defined hereinbefore);

6) C₁₋₄alkylR⁵⁹ (wherein R⁵⁹ is a group selected from pyrrolidinyl, piperazinyl, piperidinyl, imidazolidin-1-yl, azetidinyl, 1,3-dioxolan-2-yl, 1,3-dioxan-2-yl, 1,3-dithiolan-2-yl and 1,3-dithian-2-yl, which group is linked to C₁₋₄alkyl through a carbon atom and which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(Cl 3alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl)) or C₂₋₄alkylR⁶⁰ (wherein R⁶⁰ is a group selected from morpholino, thiomorpholino, azetidin-1-yl, pyrrolidin-1-yl, piperazin-1-yl and piperidino which group may bear 1 or 2 substituents selected from oxo, hydroxy, halogeno, cyano, C₁₋₃cyanoalkyl, C₁₋₃alkyl, C₁₋₃hydroxyalkyl, C₁₋₃alkoxy, C₁₋₂alkoxyC₁₋₃alkyl, C₁₋₂alkylsulphonylC₁₋₃alkyl, C₁₋₃alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, piperazinyl, piperidinyl, imidazolidinyl, azetidinyl, morpholino and thiomorpholino, which cyclic group may bear one or more substituents selected from C₁₋₃alkyl));

7) C₃₋₄alkenylR⁶¹ (wherein R⁶¹ represents R⁵⁹ or R⁶⁰ as defined hereinbefore);

8) C₃₋₄alkynylR⁶¹ (wherein R⁶¹ represents R⁵⁹ or R⁶⁰ as defined hereinbefore);

9) R²⁹ (wherein R²⁹ is as defined hereinbefore);

10) C₁₋₄alkylR²⁹ (wherein R²⁹ is as defined hereinbefore);

11) 1-R²⁹prop-1-en-3-yl or l-R²⁹but-2-en-4-yl (wherein R²⁹ is as defined hereinbefore with the proviso that when R⁵ is 1-R²⁹prop-1-en-3-yl, R²⁹ is linked to the alkenyl group via a carbon atom);

12) 1-R²⁹prop-1-yn-3-yl or 1-R²⁹but-2-yn-4-yl (wherein R²⁹ is as defined hereinbefore with the proviso that when R⁵ is 1-R²⁹prop-1-yn-3-yl, R²⁹ is linked to the alkynyl group via a carbon atom);

13) C₁₋₅alkylX⁶R²⁹ (wherein X⁶ and R²⁹ are as defined hereinbefore);

14) 1-(R²⁹X⁷)but-2-en-4-yl (wherein X⁷ and R²⁹ are as defined hereinbefore);

15) 1-(R²⁹X⁸)but-2-yn-4-yl (wherein X⁸ and R²⁹ are as defined hereinbefore);

16) C₂₋₃alkylX⁹C₁₋₃alkylR²⁹ (wherein X⁹ and R²⁹ are as defined hereinbefore);

17) C₂₋₃alkylX⁹C₁₋₃alkylR²W (wherein X⁹ and R²⁸ are as defined hereinbefore);

18) C₂₋₅alkenyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;

19) C₂₋₅alkynyl which may be unsubstituted or which may be substituted with one or more fluorine atoms or with one or two groups selected from hydroxy, fluoro, amino, C₁₋₄alkylamino, N-di(C₁₋₄alkyl)amino, aminosulphonyl, N-C₁₋₄alkylaminosulphonyl and N,N-di(C₁₋₄alkyl)aminosulphonyl;

20) C₂₋₄alkenylX⁹C₁₋₃alkylR²⁸ (wherein X⁹ and R²⁸ are as defined hereinbefore);

21) C₂₋₄alkynylX⁹C₁₋₃alkylR²⁸ (wherein X⁹ and R²⁸ are as defined hereinbefore); and

22) C₁₋₃alkylR⁵⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹— may bear one or more substituents selected from hydroxy, halogeno and amino].

According to another aspect of the present invention preferably R² represents hydroxy, halogeno, nitro, trifluoromethyl, C₁₋₃alkyl, cyano, amino or R⁵X¹— [wherein X¹ is as hereinbefore defined and R⁵ is selected from one of the following twenty groups:

1) C₁₋₃alkyl which may be unsubstituted or which may be substituted with one or more groups selected from fluoro, chloro and bromo, or C₂₋₃alkyl which may be unsubstituted or substituted with one or more groups selected from hydroxy and amino;

2) 2-(3,3-dimethylureido)ethyl, 3-(3,3-dimethylureido)propyl, 2-(3-methylureido)ethyl, 3-(3-methylureido)propyl, 2-ureidoethyl, 3-ureidopropyl, 2-(N,N-di-methylcarbamoyloxy)ethyl, 3-(N,N-dimethylcarbamoyloxy)propyl, 2-(N-methylcarbamoyloxy)ethyl, 3-(N-methylcarbamoyloxy)propyl, 2-(carbamoyloxy)ethyl, 3-(carbamoyloxy)propyl, or 2-(N-methyl-N-(butoxycarbonyl)amino)ethyl;

3) C₂₋₃alkylR¹⁶ (wherein X³ is as hereinbefore defined and R¹⁶ is a group selected from C₁₋₃alkyl, cyclopentyl, cyclohexyl, pyrrolidinyl, piperidinyl, piperazinyl, azetidinyl, imidazolidinyl and tetrahydropyranyl which group is linked to X³ through, a carbon atom and which C₁₋₃alkyl group may bear 1 or 2 substituents selected from hydroxy, halogeno and C₁₋₂alkoxy and which cyclopentyl, cyclohexyl, pyrrolidinyl, piperidinyl, piperazinyl, azetidinyl, imidazolidinyl or tetrahydropyranyl group may bear one substituent selected from oxo, hydroxy, halogeno, cyano, C₁₋₂cyanoalkyl, C₁₋₂alkyl, C₁₋₂hydroxyalkyl, C₁₋₂alkoxy, C₁₋₂alkoxyC₁₋₃alkyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));

4) C₂₋₃alkylX⁴C₂₋₃alkylX⁵R²² (wherein X⁴ and X⁵ are as hereinbefore defined and R²² represents hydrogen or C₁₋₂alkyl);

5) R²⁸ (wherein R²e is as defined hereinbefore);

6) C₁₋₃alkylR⁵⁹ (wherein R⁵⁹ is a group selected from pyrrolidinyl,-piperazinyl, piperidinyl, azetidinyl, imidazolidinyl, 1,3-dioxolan-2-yl, 1,3-dioxan-2-yl, 1,3-dithiolan-2-yl and 1,3-dithian-2-yl, which group is linked to C₁₋₃alkyl through a carbon atom and which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkoxyC₁₋₃alkyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkoxyC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino)) or C₂₋₃alkylR⁶⁰ (wherein R⁶⁰ is a group selected from morpholino, thiomorpholino, azetidin-1-yl, pyrrolidin-1-yl, piperazin-1-yl and piperidino which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkoxyC₁₋₃alkyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));

7) R²⁹ (wherein R²⁹ is as defined hereinbefore);

8) C₁₋₄alkyl R²⁹ (wherein R²⁹ is as defined hereinbefore);

9) 1-R²⁹but-2-en-4-yl (wherein R²⁹ is as defined hereinbefore);

10) 1-R²⁹but-2-yn-4-yl (wherein R²⁹ is as defined hereinbefore);

11) C₁₋₃alkylX⁶R²⁹ (wherein X⁶ and R²⁹ are as defined hereinbefore);

12) 1-(R²⁹X⁷)but-2-en-4-yl (wherein X⁷ and R²⁹ are as defined hereinbefore);

13) 1-(R²⁹X⁸)but-2-yn-4-yl (wherein X⁸ and R²⁹ are as defined hereinbefore);

14) C₂₋₃alkylX⁹C₁₋₃alkylR²⁹ (wherein X⁹ and R²⁹ are as defined hereinbefore);

15) C₂₋₃alkylX⁹C₁₋₃alkylR²⁸ (wherein X⁹ and R²¹ are as defined hereinbefore);

16) C₂₋₅alkenyl which may be unsubstituted or which may be substituted with one or more fluorine atoms or with one or two groups selected from hydroxy, fluoro, amino, C₁₋₄alkylamino, N N-di(C₁₋₄alkyl) amino, aminosulphonyl, N-C₁₋₄alkylaminosulphonyl and N,N-di(C₁₋₄alkyl)aminosulphonyl;

17) C₂₋₅alkynyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl; 18) C₂₋₃alkenylX⁹C₁₋₃alkylR²⁸ (wherein X⁹ and R²⁸ are as defined hereinbefore); 19) C₂₋₃alkynylX⁹C₁₋₃alkylR²⁸ (wherein X⁹ and R²⁸ are as defined hereinbefore); and

20) C₁₋₃al R⁴(C₁₋₃alkyl)_{q}(X⁹)ᵣR⁵⁵ (wherein X⁹, q, r, R⁵⁴ and R⁵⁵ are as defined hereinbefore);

and additionally wherein any C₁₋₅alkyl, C₂₋₅alkenyl or C₂₋₅alkynyl group in R⁵X¹′ may bear one or more substituents selected from hydroxy, halogeno and amino].

According to another aspect of the present invention more preferably R² represents hydroxy, C₁₋₃alkyl, amino or R⁵X¹— [wherein X¹ is as hereinbefore defined and R⁵ represents methyl, ethyl, benzyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl)ethyl, 2-(ethylsulphonyl)ethyl, 2-(N,N-dimethylsulphamoyl)ethyl, 2-(N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-(N,N-dimethylamino)propyl, 2-(N,N-diethylamino)ethyl, 3-(N,N-diethylamino)propyl, 2-(N-methyl-N-methylsulphonylamino)ethyl, 3-(N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperidin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-(piperidin-2-yl)propyl, (1-methylpiperidin-3-yl)methyl, (1-methylpiperidin-4-yl)methyl, (1-cyanomethylpiperidin-3-yl)methyl, (1-cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-methoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-4-yl)ethyl, 3-(1-isopropylpiperidin-2-yl)propyl, 3-(1-isopropylpiperidin-3-yl)propyl, 3-(1-isopropylpiperidin-4-yl)propyl, 2-(piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-(piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H-pyrrolidin-5-yl)methyl, (1,3-dioxolan-2-yl)methyl, 2-(1,3-dioxolan-2-yl)ethyl, 2-(2-methoxyethylamino)ethyl, 2-(N-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxyethylamino)propyl, 2-methylthiazol-4-ylmethyl, 2-acetamidothiazol-4-ylmethyl, 1-methylimidazol-2-ylmethyl, 2-(imidazol-1-yl)ethyl, 2-(2-methylimidazol-1-yl)ethyl, 2-(2-ethylimidazol-1-yl)ethyl, 3-(2-methylimidazol-1-yl)propyl, 3-(2-ethylimidazol-1-yl)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(1-methylimidazol-4-ylsulphonyl)-N-methyl)amino)ethyl, 2-((N-(3-morpholinopropylsulphonyl)-N-methyl)amino)ethyl, 2-((N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-ylmethyl, 1-(2-azetidinylethyl)piperidin-4-ylmethyl or 1-(3-azetidinylpropyl)piperidin-4-ylmethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2,S)-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(1-methylpiperazin-4-yl)-2-hydroxypropyl, (2R)-3-(1-methylpiperazin-4-yl)-2-hydroxypropyl, (2S)-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N,N-diethylamino)-2-hydroxypropyl, 3-(isopropylamino)-2-hydroxypropyl, (2R)-3-(isopropylamino)-2-hydroxypropyl, (2S)-3-(isopropylamino)-2-hydroxypropyl, 3-(N,N-diisopropylamino)-2-hydroxypropyl, (2R)-3-(N,N-diisopropylamino)-2-hydroxypropyl or (28)-3-(N,N-diisopropylamino)-2-hydroxypropyl].

According to another aspect of the present invention particularly R² represents C₁₋₃alkyl, amino or R⁵X¹— [wherein X¹ is as hereinbefore defined and R⁵ represents ethyl, benzyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl)ethyl, 2-(ethylsulphonyl)ethyl, 2-(N,N-dimethylsulphamoyl)ethyl, 2-N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-(N,N-dimethylamino)propyl, 2-(N,N-diethylamino)ethyl, 3-(N,N-diethylamino)propyl, 2-(N-methyl-N-methylsulphonylamino)ethyl, 3-N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperidin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-piperidin-2-yl)propyl, (1-methylpiperidin-3-yl)methyl, (1-methylpiperidin-4-yl)methyl, (1-cyanomethylpiperidin-3-yl)methyl, (1-cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-methoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-4-yl)ethyl, 3-(1-isopropylpiperidin-2-yl)propyl, 3-(1-isopropylpiperidin-3-yl)propyl, 3-(1-isopropylpiperidin-4-yl)propyl, 2-(piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H-pyrrolidin-5-yl)methyl, (1,3-dioxolan-2-yl)methyl, 2-(1,3-dioxolan-2-yl)ethyl, 2-(2-methoxyethylamino)ethyl, 2-(N-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxyethylamino)propyl, 2-methylthiazol-4-ylmethyl, 2-acetamidothiazol-4-ylmethyl, 1-methylimidazol-2-ylmethyl, 2-(imidazol-1-yl)ethyl, 2-(2-methylimidazol-1-yl)ethyl, 2-(2-ethylimidazol-1-yl)ethyl, 3-(2-methylimidazol-1-yl)propyl, 3-(2-ethylimidazol-1-yl)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(1-methylimidazol-4-ylsulphonyl)-N-methyl)amino)ethyl, 2-((N-(3-morpholinopropylsulphonyl)-N-methyl)amino)ethyl, 2-(N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-ylmethyl, 1-(2-azetidinylethyl)piperidin-4-ylmethyl or 1-(3-azetidinylpropyl)piperidin-4-ylmethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2S)-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(1-methylpiperazin-4-yl)-2-hydroxypropyl, (2R)-3-(1-methylpiperazin-4-yl)-2-hydroxypropyl, (2S-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N-diethylamino)-2-hydroxypropyl, 3-(isopropylamino)-2-hydroxypropyl, (2R)-3-(isopropylamino)-2-hydroxypropyl, (2S)-3-(isopropylamino)-2-hydroxypropyl, 3-(N,N-diisopropylamino)-2-hydroxypropyl, (2R)-3-(N,N-diisopropylamino)-2-hydroxypropyl or (2S)-3-(N,N-diisopropylamino)-2-hydroxypropyl].

According to another aspect of the present invention more particularly R² represents C₁₋₃alkyl, amino or R⁵X¹— [wherein X¹ is as hereinbefore defined and R⁵ represents ethyl, trifluoromethyl, 2,2,2-trifluoroethyl, 2-hydroxyethyl, 3-hydroxypropyl, 2-methoxyethyl, 3-methoxypropyl, 2-(methylsulphinyl)ethyl, 2-(methylsulphonyl)ethyl, 2-(ethylsulphinyl)ethyl, 2-(ethylsulphonyl)ethyl, 2-(N,N-dimethylsulphamoyl)ethyl, 2-(N-methylsulphamoyl)ethyl, 2-sulphamoylethyl, 2-(methylamino)ethyl, 3-(methylamino)propyl, 2-(ethylamino)ethyl, 3-(ethylamino)propyl, 2-(N,N-dimethylamino)ethyl, 3-N,N-dimethylamino)propyl, 2-(N,N-diethylamino)ethyl, 3-(N,N-diethylamino)propyl, 2-(N-methyl-N-methylsulphonylamino)ethyl, 3-(N-methyl-N-methylsulphonylamino)propyl, 2-morpholinoethyl, 3-morpholinopropyl, 2-piperidinoethyl, 3-piperidinopropyl, 2-(methylpiperidino)ethyl, 3-(methylpiperidino)propyl, 2-(ethylpiperidino)ethyl, 3-(ethylpiperidino)propyl, 2-((2-methoxyethyl)piperidino)ethyl, 3-((2-methoxyethyl)piperidino)propyl, 2-((2-methylsulphonyl)ethylpiperidino)ethyl, 3-((2-methylsulphonyl)ethylpiperidino)propyl, piperidin-3-ylmethyl, piperidin-4-ylmethyl, 2-(piperidin-3-yl)ethyl, 2-(piperidin-4-yl)ethyl, 3-(piperidin-3-yl)propyl, 3-(piperidin-4-yl)propyl, 2-(piperidin-2-yl)ethyl, 3-piperidin-2-yl)propyl, (1-methylpiperidin-3-yl)methyl, (1-methylpiperidin-4-yl)methyl, (1-cyanomethylpiperidin-3-yl)methyl, (1-cyanomethylpiperidin-4-yl)methyl, 2-(methylpiperidin-3-yl)ethyl, 2-(methylpiperidin-4-yl)ethyl, 2-(1-cyanomethylpiperidin-3-yl)ethyl, 2-(1-cyanomethylpiperidin-4-yl)ethyl, 3-(methylpiperidin-3-yl)propyl, 3-(methylpiperidin-4-yl)propyl, 3-(1-cyanomethylpiperidin-3-yl)propyl, 3-(1-cyanomethylpiperidin-4-yl)propyl, 2-(ethylpiperidin-3-yl)ethyl, 2-(ethylpiperidin-4-yl)ethyl, 3-(ethylpiperidin-3-yl)propyl, 3-(ethylpiperidin-4-yl)propyl, ((2-methoxyethyl)piperidin-3-yl)methyl, ((2-methoxyethyl)piperidin-4-yl)methyl, 2-((2-methoxyethyl)piperidin-3-yl)ethyl, 2-((2-methoxyethyl)piperidin-4-yl)ethyl, 3-((2-methoxyethyl)piperidin-3-yl)propyl, 3-((2-methoxyethyl)piperidin-4-yl)propyl, (1-(2-methylsulphonylethyl)piperidin-3-yl)methyl, (1-(2-methylsulphonylethyl)piperidin-4-yl)methyl, 2-((2-methylsulphonylethyl)piperidin-3-yl)ethyl, 2-((2-methylsulphonylethyl)piperidin-4-yl)ethyl, 3-((2-methylsulphonylethyl)piperidin-3-yl)propyl, 3-((2-methylsulphonylethyl)piperidin-4-yl)propyl, 1-isopropylpiperidin-2-ylmethyl, 1-isopropylpiperidin-3-ylmethyl, 1-isopropylpiperidin-4-ylmethyl, 2-(1-isopropylpiperidin-2-yl)ethyl, 2-(1-isopropylpiperidin-3-yl)ethyl, 2-(1-isopropylpiperidin-4-yl)ethyl, 3-(1-isopropylpiperidin-2-yl)propyl, 3-(1-isopropylpiperidin-3-yl)propyl, 3-(1-isopropylpiperidin-4-yl)propyl, 2-(piperidin-4-yloxy)ethyl, 3-(piperidin-4-yloxy)propyl, 2-(1-(cyanomethyl)piperidin-4-yloxy)ethyl, 3-(1-(cyanomethyl)piperidin-4-yloxy)propyl, 2-(1-(2-cyanoethyl)piperidin-4-yloxy)ethyl, 3-(1-(2-cyanoethyl)piperidin-4-yloxy)propyl, 2-(piperazin-1-yl)ethyl, 3-(piperazin-1-yl)propyl, (pyrrolidin-2-yl)methyl, 2-(pyrrolidin-1-yl)ethyl, 3-(pyrrolidin-1-yl)propyl, (2-oxo-tetrahydro-2H-pyrrolidin-5-yl)methyl, 5(R)-(2-oxo-tetrahydro-2H-pyrrolidin-5-yl)methyl, (5S)-(2-oxo-tetrahydro-2H-pyrrolidin-5-yl)methyl, (1,3-dioxolan-2-yl)methyl, 2-(1,3-dioxolan-2-yl)ethyl, 2-(2-methoxyethylamino)ethyl, 2-(N-(2-methoxyethyl)-N-methylamino)ethyl, 2-(2-hydroxyethylamino)ethyl, 3-(2-methoxyethylamino)propyl, 3-(N-(2-methoxyethyl)-N-methylamino)propyl, 3-(2-hydroxyethylamino)propyl, 2-(1,2,3-triazol-1-yl)ethyl, 2-(1,2,3-triazol-2-yl)ethyl, 2-(1,2,4-triazol-1-yl)ethyl, 2-(1,2,4-triazol-4-yl)ethyl, 4-pyridylmethyl, 2-(4-pyridyl)ethyl, 3-(4-pyridyl)propyl, 2-(4-pyridyloxy)ethyl, 2-(4-pyridylamino)ethyl, 2-(4-oxo-1,4-dihydro-1-pyridyl)ethyl, 2-(2-oxo-imidazolidin-1-yl)ethyl, 3-(2-oxo-imidazolidin-1-yl)propyl, 2-thiomorpholinoethyl, 3-thiomorpholinopropyl, 2-(1,1-dioxothiomorpholino)ethyl, 3-(1,1-dioxothiomorpholino)propyl, 2-(2-methoxyethoxy)ethyl, 2-(4-methylpiperazin-1-yl)ethyl, 3-(4-methylpiperazin-1-yl)propyl, 3-(methylsulphinyl)propyl, 3-(methylsulphonyl)propyl, 3-(ethylsulphinyl)propyl, 3-(ethylsulphonyl)propyl, 2-(5-methyl-1,2,4-triazol-1-yl)ethyl, morpholino, 2-((N-(3-morpholinopropylsulphonyl)-N-methyl)amino)ethyl, 2-(N-methyl-N-4-pyridyl)amino)ethyl, 3-(4-oxidomorpholino)propyl, 2-(2-(4-methylpiperazin-1-yl)ethoxy)ethyl, 3-(2-(4-methylpiperazin-1-yl)ethoxy)propyl, 2-(2-morpholinoethoxy)ethyl, 3-(2-morpholinoethoxy)propyl, 2-(tetrahydropyran-4-yloxy)ethyl, 3-(tetrahydropyran-4-yloxy)propyl, 2-((2-(pyrrolidin-1-yl)ethyl)carbamoyl)vinyl, 3-((2-(pyrrolidin-1-yl)ethyl)carbamoyl)prop-2-en-1-yl, 1-(2-pyrrolidinylethyl)piperidin-4-ylmethyl, 1-(3-pyrrolidinylpropyl)piperidin-4-ylmethyl, 1-(2-piperidinylethyl)piperidin-4-ylmethyl, 1-(3-piperidinylpropyl)piperidin-4-ylmethyl, 1-(2-morpholinoethyl)piperidin-4-ylmethyl, 1-(3-morpholinopropyl)piperidin-4-ylmethyl, 1-(2-thiomorpholinoethyl)piperidin-4-ylmethyl, 1-(3-thiomorpholinopropyl)piperidin-4-ylmethyl, 1-(2-azetidinylethyl)piperidin-4-ylmethyl or 1-(3-azetidinylpropyl)piperidin-4-ylmethyl, 3-morpholino-2-hydroxypropyl, (2R)-3-morpholino-2-hydroxypropyl, (2S)-3-morpholino-2-hydroxypropyl, 3-piperidino-2-hydroxypropyl, (2R)-3-piperidino-2-hydroxypropyl, (2S)-3-piperidino-2-hydroxypropyl, 3-pyrrolidin-1-yl-2-hydroxypropyl, (2R)-3-pyrrolidin-1-yl-2-hydroxypropyl, (2S)-3-pyrrolidin-1-yl-2-hydroxypropyl, 3-(1-methylpiperazin-4-yl)-2-hydroxypropyl, (2R)-3-(1-methylpiperazin-4-yl)-2-hydroxypropyl, (2S)-3-(1-methylpiperazin-4-yl)-2-hydroxypropyl, 3-(N,N-diethylamino)-2-hydroxypropyl, (2R)-3-(N,N-diethylamino)-2-hydroxypropyl, (2S)-3-(N,N-diethylamino)-2-hydroxypropyl, 3-(isopropylamino)-2-hydroxypropyl, (2R)-3-(isopropylamino)-2-hydroxypropyl, (2S)-3-(isopropylamino)-2-hydroxypropyl, 3-(N,N-diisopropylamino)-2-hydroxypropyl, (2R)-3-(N,N-diisopropylamino)-2-hydroxypropyl or (2S)-3-(N,N-diisopropylamino)-2-hydroxypropyl].

In another aspect R² represents ethoxy, trifluoromethoxy, 2,2,2-trifluoroethoxy, 2-hydroxyethoxy, 3-hydroxypropoxy, 2-methoxyethoxy, 3-methoxypropoxy, 2-(methylsulphinyl)ethoxy, 2-(methylsulphonyl)ethoxy, 2-(ethylsulphinyl)ethoxy, 2-(ethylsulphonyl)ethoxy, 2-(N,N-dimethylsulphamoyl)ethoxy, 2-(N-methylsulphamoyl)ethoxy, 2-sulphamoylethoxy, 2-(methylamino)ethoxy, 3-(methylamino)propoxy, 2-(ethylamino)ethoxy, 3-(ethylamino)propoxy, 2-(N,N-dimethylamino)ethoxy, 3-(N,N-dimethylamino)propoxy, 2-(N,N-diethylamino)ethoxy, 3-(N,N-diethylamino)propoxy, 2-(N-methyl-N-methylsulphonylamino)ethoxy, 3-(N-methyl-N-methylsulphonylamino)propoxy, 2-morpholinoethoxy, 3-morpholinopropoxy, 2-piperidinoethoxy, 3-piperidinopropoxy, 2-(methylpiperidino)ethoxy, 3-(methylpiperidino)propoxy, 2-(ethylpiperidino)ethoxy, 3-(ethylpiperidino)propoxy, 2-((2-methoxyethyl)piperidino)ethoxy, 3-((2-methoxyethyl)piperidino)propoxy, 2-((2-methylsulphonyl)ethylpiperidino)ethoxy, 3-((2-methylsulphonyl)ethylpiperidino)propoxy, piperidin-3-ylmethoxy, piperidin-4-ylmethoxy, 2-(piperidin-3-yl)ethoxy, 2-(piperidin-4-yl)ethoxy, 3-(piperidin-3-yl)propoxy, 3-piperidin-4-yl)propoxy, 2-(piperidin-2-yl)ethoxy, 3-(piperidin-2-yl)propoxy, (1-methylpiperidin-3-yl)methoxy, (1-methylpiperidin-4-yl)methoxy, (1-cyanomethylpiperidin-3-yl)methoxy, (1-cyanomethylpiperidin-4-yl)methoxy, 2-(methylpiperidin-3-yl)ethoxy, 2-(methylpiperidin-4-yl)ethoxy, 2-(1-cyanomethylpiperidin-3-yl)ethoxy, 2-(1-cyanomethylpiperidin-4-yl)ethoxy, 3-(methylpiperidin-3-yl)propoxy, 3-(methylpiperidin-4-yl)propoxy, 3-(1-cyanomethylpiperidin-3-yl)propoxy, 3-(1-cyanomethylpiperidin-4-yl)propoxy, 2-(ethylpiperidin-3-yl)ethoxy, 2-(ethylpiperidin-4-yl)ethoxy, 3-(ethylpiperidin-3-yl)propoxy, 3-(ethylpiperidin-4-yl)propoxy, ((2-methoxyethyl)piperidin-3-yl)methoxy, ((2-methoxyethyl)piperidin-4-yl)methoxy, 2-((2-methoxyethyl)piperidin-3-yl)ethoxy, 2-((2-methoxyethyl)piperidin-4-yl)ethoxy, 3-((2-methoxyethyl)piperidin-3-yl)propoxy, 3-((2-methoxyethyl)piperidin-4-yl)propoxy, (1-(2-methylsulphonylethyl)piperidin-3-yl)methoxy, (1-(2-methylsulphonylethyl)piperidin-4-yl)methoxy, 2-((2-methylsulphonylethyl)piperidin-3-yl)ethoxy, 2-((2-methylsulphonylethyl)piperidin-4-yl)ethoxy, 3-((2-methylsulphonylethyl)piperidin-3-yl)propoxy, 3-((2-methylsulphonylethyl)piperidin-4-yl)propoxy, 1-isopropylpiperidin-2-ylmethoxy, 1-isopropylpiperidin-3-ylmethoxy, 1-isopropylpiperidin-4-ylmethoxy, 2-(1-isopropylpiperidin-2-yl)ethoxy, 2-(1-isopropylpiperidin-3-yl)ethoxy, 2-(1-isopropylpiperidin-4-yl)ethoxy, 3-(1-isopropylpiperidin-2-yl)propoxy, 3-(1-isopropylpiperidin-3-yl)propoxy, 3-(1-isopropylpiperidin-4-yl)propoxy, 2-(piperidin-4-yloxy)ethoxy, 3-(piperidin-4-yloxy)propoxy, 2-(1-(cyanomethyl)piperidin-4-yloxy)ethoxy, 3-(1-(cyanomethyl)piperidin-4-yloxy)propoxy, 2-(1-(2-cyanoethyl)piperidin-4-yloxy)ethoxy, 3-(1-(2-cyanoethyl)piperidin-4-yloxy)propoxy, 2-(piperazin-1-yl)ethoxy, 3-(piperazin-1-yl)propoxy, (pyrrolidin-2-yl)methoxy, 2-(pyrrolidin-1-yl)ethoxy, 3-(pyrrolidin-1-yl)propoxy, (2-oxo-tetrahydro-2H-pyrrolidin-5-yl)methoxy, 5(R)-(2-oxo-tetrahydro-2H-pyrrolidin-5-yl)methoxy, (5S)-(2-oxo-tetrahydro-2H-pyrrolidin-5-yl)methoxy, (1,3-dioxolan-2-yl)methoxy, 2-(1,3-dioxolan-2-yl)ethoxy, 2-(2-methoxyethylamino)ethoxy, 2-(N-(2-methoxyethyl)-N-methylamino)ethoxy, 2-(2-hydroxyethylamino)ethoxy, 3-(2-methoxyethylamino)propoxy, 3-(N-(2-methoxyethyl)-N-methylamino)propoxy, 3-(2-hydroxyethylamino)propoxy, 2-(1,2,3-triazol-1-yl)ethoxy, 2-(1,2,3-triazol-2-yl)ethoxy, 2-(1,2,4-triazol-1-yl)ethoxy, 2-(1,2,4-triazol-4-yl)ethoxy, 4-pyridylmethyl, 2-(4-pyridyl)ethyl, 4-pyridylmethoxy, 2-(4-pyridyl)ethoxy, 3-(4-pyridyl)propoxy, 2-(4-pyridyloxy)ethoxy, 2-(4-pyridylamino)ethoxy, 2-(4-oxo-1,4-dihydro-1-pyridyl)ethoxy, 2-(2-oxo-imidazolidin-1-yl)ethoxy, 3-(2-oxo-imidazolidin-1-yl)propoxy, 2-thiomorpholinoethoxy, 3-thiomorpholinopropoxy, 2-(1,1-dioxothiomorpholino)ethoxy, 3-(1,1-dioxothiomorpholino)propoxy, 2-(2-methoxyethoxy)ethoxy, 2-(4-methylpiperazin-1-yl)ethoxy, 3-(4-methylpiperazin-1-yl)propoxy, 3-(methylsulphinyl)propoxy, 3-(methylsulphonyl)propoxy, 3-(ethylsulphinyl)propoxy, 3-(ethylsulphonyl)propoxy, 2-(5-methyl-1,2,4-triazol-1-yl)ethoxy, 2-((N-(3-morpholinopropylsulphonyl)-N-methyl)amino)ethoxy, 2-((N-methyl-N-4-pyridyl)amino)ethoxy, 3-(4-oxidomorpholino)propoxy, 2-(2-(4-methylpiperazin-1-yl)ethoxy)ethoxy, 3-(2-(4-methylpiperazin-1-yl)ethoxy)propoxy, 2-(2-morpholinoethoxy)ethoxy, 3-(2-morpholinoethoxy)propoxy, 2-(tetrahydropyran-4-yloxy)ethoxy, 3-(tetrahydropyran-4-yloxy)propoxy, 2-((2-(pyrrolidin-1-yl)ethyl)carbamoyl)vinyl, 3-((2-(pyrrolidin-1-yl)ethyl)carbamoyl)prop-2-en-1-yloxy, 1-(2-pyrrolidinylethyl)piperidin-4-ylmethoxy, 1-(3-pyrrolidinylpropyl)piperidin-4-ylmethoxy, 1-(2-piperidinylethyl)piperidin-4-ylmethoxy, 1-(3-piperidinylpropyl)piperidin-4-ylmethoxy, 1-(2-morpholinoethyl)piperidin-4-ylmethoxy, 1-(3-morpholinopropyl)piperidin-4-ylmethoxy, 1-(2-thiomorpholinoethyl)piperidin-4-ylmethoxy, 1-(3-thiomorpholinopropyl)piperidin-4-ylmethoxy, 1-(2-azetidinylethyl)piperidin-4-ylmethoxy or 1-(3-azetidinylpropyl)piperidin-4-ylmethoxy, 3-morpholino-2-hydroxypropoxy, (2R)-3-morpholino-2-hydroxypropoxy, (2S)-3-morpholino-2-hydroxypropoxy, 3-piperidino-2-hydroxypropoxy, (2R)-3-piperidino-2-hydroxypropoxy, (2S)-3-piperidino-2-hydroxypropoxy, 3-pyrrolidin-1-yl-2-hydroxypropoxy, (2R)-3-pyrrolidin-1-yl-2-hydroxypropoxy, (28)-3-pyrrolidin-1-yl-2-hydroxypropoxy, 3-(i-methylpiperazin-4-yl)-2-hydroxypropoxy, (2R)-3-(1-methylpiperazin-4-yl)-2-hydroxypropoxy, (25-3-(1-methylpiperazin-4-yl)-2-hydroxypropoxy, 3-(N,N-diethylamino)-2-hydroxypropoxy, (2R)-3-(N,N-diethylamino)-2-hydroxypropoxy, (2S)-3-(N,N-diethylamino)-2-hydroxypropoxy, 3-(isopropylamino)-2-hydroxypropoxy, (2R)-3-(isopropylamino)-2-hydroxypropoxy, (2S)-3-(isopropylamino)-2-hydroxypropoxy, 3-(N,N-diisopropylamino)-2-hydroxypropoxy, (2R)-3-(N,N-diisopropylamino)-2-hydroxypropoxy or (2S)-3-(N,N-diisopropylamino)-2-hydroxypropoxy.

Where one of the R² substituents is R⁵X¹— the substituent R⁵X¹— is preferably at the position of ring C which would correspond to either the 6- or 7-position of a 10-membered bicyclic moiety which is attached to Z at the 4-position.

In another aspect of the present invention there is provided the use of compounds of the formula I, as defined hereinbefore with the proviso that Z is-O—, or a salt thereof, or a prodrug thereof for example an ester or an amide, in the manufacture of a medicament for use in the production of an antiangiogenic and/or vascular permeability reducing effect in warm-blooded animals such as humans.

In another aspect of the present invention there is provided the use of compounds of the formula Ia:

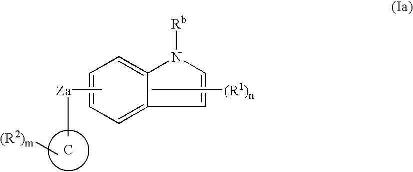

wherein ring C, R^{b}, R¹, R², m and n are as defined hereinbefore and Za represents —O—, —CH₂—, —NH— or —S—; or a salt thereof, or a prodrug thereof for example an ester or an amide, in the manufacture of a medicament for use in the production of an antiangiogenic and/or vascular permeability reducing effect in warm-blooded animals such as humans.

In another aspect of the present invention there is provided the use of compounds of the formula Ib:

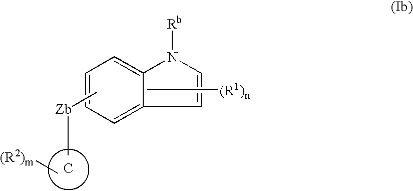

wherein ring C, R^{b}, R¹, R², m and n are as defined hereinbefore and Za represents —O—, —NH— or —S—; or a salt thereof, or a prodrug thereof for example an ester or an amide, in the manufacture of a medicament for use in the production of an antiangiogenic and/or vascular permeability reducing effect in warm-blooded animals such as humans.

In another aspect of the present invention there is provided the use of compounds of the formula Ic:

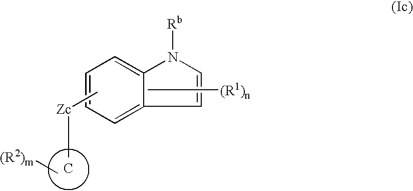

wherein ring C, R^{b}, R¹, R², m and n are as defined hereinbefore and Zc represents —O—; or a salt thereof, or a prodrug thereof for example an ester or an amide, in the manufacture of a medicament for use in the production of an antiangiogenic and/or vascular permeability reducing effect in warm-blooded animals such as humans.

In another aspect of the present invention there is provided the use of compounds of the formula Ib as defined hereinbefore with the proviso that when ring C is

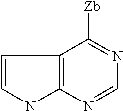

wherein Zb is as defined hereinbefore, (but Zb is not a part of ring C, it is shown for the purposes of clarity), at least one R² does not have a value selected from hydrogen, halogeno, C₁₋₄alkyl, C₁₋₄alkoxy and NR^{c}R^{d} (wherein each of R^{d} and R^{d} independently represents hydrogen, C₁₋₄alkyl or phenyl which phenyl may bear 1-3 substituents selected from halogeno, trifluoromethyl, C₁₋₄alkyl and C₁₋₄alkoxy); or a salt thereof, or a prodrug thereof for example an ester or an amide, in the manufacture of a medicament for use in the production of an antiangiogenic and/or vascular permeability reducing effect in warm-blooded animals such as humans.

According to another aspect of the present invention there is provided a compound of the formula I as defined hereinbefore and salts thereof, and prodrugs thereof for example esters and amides.

According to another aspect of the present invention there is provided a compound of the formula I as defined hereinbefore and salts thereof, and prodrugs thereof for example esters and amides with the proviso that Z is —O—.

According to another aspect of the present invention there is provided a compound of the formula I^{I} as defined hereinbefore and salts thereof, and prodrugs thereof for example esters, amides and sulphides, preferably esters and amides.

According to another aspect of the present invention there is provided a compound of the formula Ia as defined hereinbefore and salts thereof, and prodrugs thereof for example esters and amides.

According to another aspect of the present invention there is provided a compound of the formula Ib as defined hereinbefore and salts thereof, and prodrugs thereof for example esters and amides.

According to another aspect of the present invention there is provided a compound of the formula Ic as defined hereinbefore and salts thereof, and prodrugs thereof for example esters and amides.

According to another aspect of the present invention there is provided a compound of the formula Ib as defined hereinbefore with the proviso that if Z is —NH— then: at least one R² is not selected from hydrogen, chloro, bromo, methyl, phenyl(hydroxymethyl), dimethylamino, methylsulphanyl, methylsulphinyl, methylsulphonyl and hydroxycyclohexylamino;

X¹ is not selected from —CH₂—, a direct bond and —C(O)NR⁷—; and

where R² is a group R⁵-X¹ and X¹ is —NR⁶C(O)— or —NR⁹SO₂—, R⁵ does not contain an alkenyl or alkynyl moiety;

and salts thereof, and prodrugs thereof for example esters and amides.

According to another aspect of the present invention there is provided a compound of the formula Ib as defined hereinbefore with the proviso that if Z is —NH— then: at least one R² is not selected from hydrogen, chloro, bromo, methyl, phenyl(hydroxymethyl), dimethylamino, methylsulphanyl, methylsulphinyl, methylsulphonyl and hydroxycyclohexylamino;

X¹ is not selected from —CH₂—, a direct bond and —C(O)NR⁷—; and

where R² is a group R⁵-X¹ and X¹ is —NR⁶C(O)— or —NR⁹SO₂—, Re does not contain an alkenyl or alkynyl moiety;

and with the further proviso that when ring C is

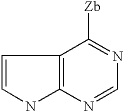

wherein Zb is as defined hereinbefore, (but Zb is not a part of ring C, it is shown for the purposes of clarity), at least one R² does not have a value selected from hydrogen, halogeno, C₁₋₄alkyl; C₁₋₄alkoxy and NR^{c}R^{d} (wherein each of R^{c} and R^{d} independently represents hydrogen, C₁₋₄alkyl or phenyl which phenyl may bear 1-3 substituents selected from halogeno, trifluoromethyl, C₁₋₄alkyl and C₁₋₄alkoxy);

and salts thereof, and prodrugs thereof for example esters and amides.

According to one aspect of the present invention preferred examples are:

1-(4-fluoroindol-5-yloxy)-4-(4-pyridylmethyl)phthalazine,

1-(indol-6-yloxy)-4-(4-pyridylmethyl)phthalazine,

1-(2-methylindol-6-yloxy)-4-(4-pyridylmethyl)phthalazine,

4-(4-fluoro-2-methylindol-5-yloxy)thieno[3,2-d]pyrimidine and

1-(4-fluoro-2-methylindol-5-yloxy)-4-(4-pyridylmethyl)phthalazine, and salts thereof.

In another aspect preferred compounds of the present invention are

4-(4-fluoro-2-methylindol-5-yloxy)thieno[3,2-d]pyrimidine and

1-(4-fluoro-2-methylindol-5-yloxy)-4-(4-pyridylmethyl)phthalazine and salts thereof.

For the avoidance of doubt it is to be understood that where in this specification a group is qualified by ‘hereinbefore defined’ or ‘defined hereinbefore’ the said group encompasses the first occurring and broadest definition as well as each and all of the preferred definitions for that group.

In this specification unless stated otherwise the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-6 carbon atoms, preferably 1-4 carbon atoms. The term “alkoxy” as used herein, unless stated otherwise includes “alkyl”-O— groups in which “alkyl” is as hereinbefore defined. The term “aryl” as used herein unless stated otherwise includes reference to a C₆₋₁₀ aryl group which may, if desired, carry one or more substituents selected from halogeno, alkyl, alkoxy, nitro, trifluoromethyl and cyano, (wherein alkyl and alkoxy are as hereinbefore defined). The term “aryloxy” as used herein unless otherwise stated includes “aryl”-O-groups in which “anyl” is as hereinbefore defined. The term “sulphoniyloxy” as used herein refers to alkylsulphonyloxy and arylsulphonyloxy groups in which “alkyl” and “aryl” are as hereinbefore defined. The term “alkanoyl” as used herein unless otherwise stated includes formyl and alkylC═O groups in which “alkyl” is as defined hereinbefore, for example C₂alkanoyl is ethanoyl and refers to CH₃C═O, C₁alkanoyl is formyl and refers to CHO. In this specification unless stated otherwise the term “alkenyl” includes both straight and branched chain alkenyl groups but references to individual alkenyl groups such as 2-butenyl are specific for the straight chain version only. Unless otherwise stated the term “alkenyl” advantageously refers to chains with 2-5 carbon atoms, preferably 3-4 carbon atoms. In this specification unless stated otherwise the term “allknyl” includes both straight and branched chain alkynyl groups but references to individual alkynyl groups such as 2-butynyl are specific for the straight chain version only. Unless otherwise stated the term “alkynyl” advantageously refers to chains with 2-5 carbon atoms, preferably 3-4 carbon atoms. Unless stated otherwise the term “haloalkyl” refers to an alkyl group as defined hereinbefore which bears one or more halogeno groups, such as for example trifluoromethyl.

For the avoidance of any doubt, where R² has a value of substituted or unsubstituted C₁₋₅alkyl, R² has been selected from C₁₋₃alkyl or from a group R⁵X¹ wherein X¹ is a direct bond or —CH₂— and R⁵ is C₁₋₅alkyl which may be unsubstituted or which maybe substituted with one or more groups selected from hydroxy, fluoro, chloro, bromo and amino.

Within the present invention it is to be understood that a compound of the formula I or a salt thereof may exhibit the phenomenon of tautomerism and that the formulae drawings within this specification can represent only one of the possible tautomeric forms. It is to be understood that the invention encompasses any tautomeric form which inhibits VEGF receptor tyrosine kinase activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It will be appreciated that compounds of the formula I or a salt thereof may possess an asymmetric carbon atom. Such an asymmetric carbon atom is also involved in the tautomerism described above, and it is to be understood that the present invention encompasses any chiral form (including both pure enantiomers, scalemic and racemic mixtures) as well as any tautomeric form which inhibits VEGF receptor tyrosine kinase activity, and is not to be limited merely to any one tautomeric form or chiral form utilised within the formulae drawings. It is to be understood that the invention encompasses all optical and diastereomers which inhibit VEGF receptor tyrosine kinase activity. It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ration 50:50.

It is also to be understood that certain compounds of the formula I and salts thereof can exist in solvated as well as unsolvated forms such as, for example, hydrated forms. It is to be understood that the invention encompasses all such solvated forms which inhibit VEGF receptor tyrosine kinase activity.

For the avoidance of any doubt, it is to be understood that when X¹ is, for example, a group of formula —NR⁶C(O)—, it is the nitrogen atom bearing the R⁶ group which is attached to ring C and the carbonyl (C(O)) group is attached to R⁵, whereas when X¹ is, for example, a group of formula —C(O)NR⁷—, it is the carbonyl group which is attached to ring C and the nitrogen atom bearing the R⁷ group is attached to R⁵. A similar convention applies to the other two atom X¹ linking groups such as —NR⁹SO₂— and —SO₂NR⁸—. When X¹ is NR¹⁰ it is the nitrogen atom bearing the R¹⁰ group which is linked to ring C and to R⁵. An analogous convention applies to other groups. It is further to be understood that when XI represents —NR¹⁰ and R¹⁰ is C₁₋₃alkoxyC₂₋₃alkyl it is the C₂₋₃alkyl moiety which is linked to the nitrogen atom of X¹ and an analogous convention applies to other groups.

For the avoidance of any doubt, it is to be understood that in a compound of the formula I when R⁵ is, for example, a group of formula C₁₋₃alkylX⁹C₁₋₃alkylR²⁹, it is the terminal C₁₋₃alkyl moiety which is linked to X¹, similarly when R⁵ is, for example, a group of formula C₂₋₅alkenylR²⁸ it is the C₂₋₅alkenyl moiety which is linked to X¹ and an analogous convention applies to other groups. When R⁵ is a group 1-9prop-1-en-3-yl it is the first carbon to which the group R²⁹ is attached and it is the third carbon which is linked to X¹ and an analogous convention applies to other groups.

For the avoidance of any doubt, it is to be understood that in a compound of the formula I when R⁵ is, for example, R²⁸ and R²⁸ is a pyrrolidinyl ring which bears a group —(—O—)_{f}(C₁₋₄alkyl)_{g}ringD, it is the —O— or C₁₋₄alkyl which is linked to the pyrrolidinyl ring, unless f and g are both 0 when it is ring D which is linked to the pyrrolidinyl ring and an analogous convention applies to other groups.

For the avoidance of any doubt, it is to be understood that when R²⁹ carries a C₁₋₄aminoal]-yl substituent it is the C₁₋₄alkyl moiety which is attached to R whereas when R²⁹ carries a C₁₋₄alkylamino substituent it is the amino moiety which is attached to R²⁹ and an analogous convention applies to other groups.

For the avoidance of any doubt, it is to be understood that when R²⁸ carries a C₁₋₄alkoxyC₁₋₄alkyl substituent it is the C₁₋₄alkyl moiety which is attached to R²⁸ and an analogous convention applies to other groups.

For the avoidance of any doubt, it is to be understood that when R¹ is —C₁₋₅alkyl(ring B) it is the alkyl chain which is linked to the indole group and ring B is attached to the alkyl chain and an analogous convention applies to other groups.

For the avoidance of any doubt, it is to be understood that when R^{b} is C₂-5alkenylaminoC₁₋₄alkyl, it is the C₁₋₄alkyl group which is, linked to the nitrogen atom of the 5-membered ring and an analogous convention applies to other groups.

The present invention relates to the compounds of formula I as hereinbefore defined as well as to the salts thereof Salts for use in pharmaceutical compositions will be pharmaceutically acceptable salts, but other salts may be useful in the production of the compounds of formula I and their pharmaceutically acceptable salts. Pharmaceutically acceptable salts of the invention may, for example, include acid addition salts of the compounds of formula I as hereinbefore defined which are sufficiently basic to form such salts. Such acid addition salts include for example salts with inorganic or organic acids affording pharmaceutically acceptable anions such as with hydrogen halides (especially hydrochloric or hydrobromic acid of which hydrochloric acid is particularly preferred) or with sulphuric or phosphoric acid, or with trifluoroacetic, citric or maleic acid. In addition where the compounds of formula I are sufficiently acidic, pharmaceutically acceptable salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, an ammonium salt or for example a salt with methylamine, dimethylamine, trimethylamine, piperidine, morpholine or tris-(2-hydroxyethyl)amine.

A compound of the formula I, or salt thereof, and other compounds of the invention (as hereinafter defined) may be prepared by any process known to be applicable to the preparation of chemically-related compounds. Such processes include, for example, those illustrated in International Patent Application Publicaiton No. WO 00/47212 (Application No. PCT/GB00/00373). Such processes also include, for example, solid phase synthesis. Such processes, are provided as a further feature of the invention and are as described hereinafter. Necessary starting materials may be obtained by standard procedures of organic chemistry. The preparation of such starting materials is described within the accompanying non-limiting Examples. Alternatively necessary starting materials are obtainable by analogous procedures to those illustrated which are within the ordinary skill of an organic chemist.

Thus, the following processes (a) to (f) and (i) to (vi) constitute further features of the present invention.

Synthesis of Compounds of Formula I

(a) Compounds of the formula I and salts thereof may be prepared by the reaction of a compound of the formula III:

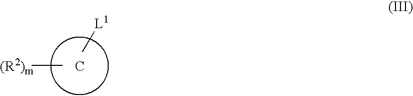

(wherein ring C, R² and m are as defined hereinbefore and L¹ is a displaceable moiety), with a compound of the formula IV:

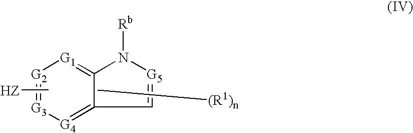

(wherein R^{b}, R¹, G₁, G₂, G₃, G₄, G₅, Z and n are as defined hereinbefore) to obtain compounds of the formula I and salts thereof. A convenient displaceable moiety L¹ is, for example, a halogeno, alkoxy (preferably C₁₋₄alkoxy), aryloxy, alkylsulphanyl, arylsulphanyl, alkoxyalkylsulphanyl or sulphonyloxy group, for example a chloro, bromo, methoxy, phenoxy, methylsulphanyl, 2-methoxyethylsulphanyl, methanesulphonyloxy or toluene-4-sulphonyloxy group.

The reaction is advantageously effected in the presence of a base. When Z is —O— such a base is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, tetramethylguanidine or for example, an alkali metal or alkaline earth metal carbonate or hydroxide, for example sodium carbonate, potassium carbonate, cesium carbonate, calcium carbonate, sodium hydroxide or potassium hydroxide. alternatively such a base is, for example, an alkali metal hydride, for example sodium hydride, or an alkali metal or alkaline earth metal amide, for example sodium amide, sodium is(trimethylsilyl)amide, potassium amide or potassium bis(trimethylsilyl)amide. The reaction is preferably effected in the presence of an inert solvent or diluent, for example an ether such as tetrahydrofuran or 1,4-dioxan, an aromatic hydrocarbon solvent such as toluene, or a dipolar aprotic solvent such as N,N-dimethylformamide, N N-dimethylacetamide, N-methylpyrrolidin-2-one or dimethyl sulphoxide. The reaction is conveniently effected at a temperature in the range, for example, 10 to 150° C., preferably in the range 20 to 110° C.

When Z is —NH— the reaction is advantageously effected in the presence of either an acid or a base. Such an acid is for example, an anhydrous inorganic acid such as hydrochloric acid, in the presence of a protic solvent or diluent, for example an alcohol or ester such as methanol, ethanol, 2-propanol, 2-pentanol.

When it is desired to obtain the acid salt, the free base may be treated with an acid such as a hydrogen halide, for example hydrogen chloride, sulphuric acid, a sulphonic acid, for example methane sulphonic acid, or a carboxylic acid, for example acetic or citric acid, using a conventional procedure.

(b) Production of those compounds of formula I and salts thereof wherein at least one R² is R⁵X¹ wherein R⁵ is as defined hereinbefore and X¹ is —O—, —S—, —OC(O)— or —NR¹⁰— (wherein R¹⁰ independently represents hydrogen, C₁₋₃alkyl or C₁₋₃alkoxyC₂₋₃alkyl) can be achieved by the reaction, conveniently in the presence of a base (as defined hereinbefore in process (a)) of a compound of the formula V:

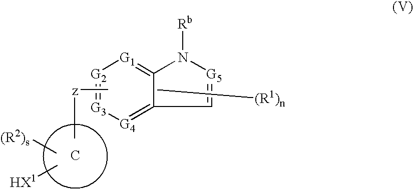

(wherein ring C, R^{b}, Z, G₁, G₂, G₃, G₄, G₅, R¹, R² and n are as hereinbefore defined and X¹ is as hereinbefore defined in this section and s is 0 or 1) with a compound of formula VI:

R⁵-L¹ (VI)

(wherein R⁵ and L¹ are as hereinbefore defined), L¹ is a displaceable moiety for example a halogeno or sulphonyloxy group such as a bromo, methanesulphonyloxy or toluene-4-sulphonyloxy group, or L¹ may be generated in situ from an alcohol under standard Mitsunobu conditions (“Organic Reactions”, John Wiley & Sons Inc, 1992, vol 42, chapter 2, David L Hughes). The reaction is preferably effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 50° C.

(c) Compounds of the formula I and salts thereof wherein at least one R² is R⁵X¹ wherein R⁵ is as defined hereinbefore and X¹ is —O—, —S—, —OC(O)— or NR¹⁰ (wherein R¹⁰ represents hydrogen, C₁₋₃alkyl or C₁₋₃alkoxyC₂₋₃alkyl) may be prepared by the reaction of a compound of the formula VII:

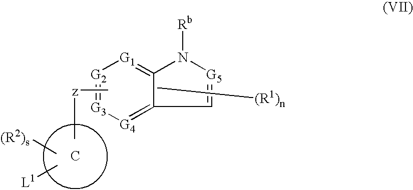

with a compound of the formula VIII:

R⁵—X—H (VIII)

(wherein ling C, L¹, R^{b}, R¹, R², R⁵, Z, G₁, G₂, G₃, G₄, G₅, n and s are all as hereinbefore defined and X¹ is as hereinbefore defined in this section). The reaction may conveniently be effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 100° C.

(d) Compounds of the formula I and salts thereof wherein at least one R² is R⁵X¹ wherein X¹ is as defined hereinbefore and R⁵ is C₁₋₅alkylR⁶², wherein R⁶² is selected from one of the following nine groups:

1) X¹⁰C₁₋₃alkyl (wherein X¹⁰ represents —O—, —S—, —SO₂—, —NR⁶³C(O)— or —NR⁶⁴SO₂— (wherein R⁶³ and R⁶⁴ which may be the same or different are each hydrogen, C₁₋₃alkyl or C₁₋₃alkoxyC₂₋₃alkyl);

2) NR⁶⁵R⁶⁶ (wherein R⁶⁵ and R⁶⁶ which may be the same or different are each hydrogen, C₁₋₃alkyl or C₁₋₃alkoxyC₂-3alkyl);

3) X¹¹C₁₋₅alkylX⁵R²² (wherein X¹ represents —O—, —S—, —SO₂—, —NR⁶⁷C(O)—, —NR⁶⁸SO₂— or —NR⁶⁹ (wherein R⁶⁷, R⁶⁸, and R⁶⁹ which may be the same or different are each hydrogen, C₁₋₃alkyl or C₁₋₃alkoxyC₂₋₃alkyl) and X⁵ and R²² are as defined hereinbefore);

4) R²⁸ (wherein R²⁸ is as defined hereinbefore);

5) X¹²R²⁹ (wherein X¹² represents —O—, —S—, —SO₂—, —NR⁷⁰C(O)—, —NR⁷¹SO₂—, or NR⁷²— (wherein R⁷⁰, R⁷¹, and R⁷² which may be the same or different are each hydrogen, C₁₋₃alkyl or C₁₋₃alkoxyC₂₋₃alkyl) and R²⁹ is as defined hereinbefore); and

6) X¹³C₁₋₃alkylR²⁹ (wherein X¹³ represents —O—, —S—, —SO₂—, —NR⁷³C(O)—, —NR⁷⁴SO₂— or —NR⁷⁵— (wherein R⁷³, R⁷⁴ and R⁷⁵ each independently represents hydrogen, C₁₋₃alkyl or C₁₋₃alkoxyC₂₋₃alkyl) and R²⁹ is as defined hereinbefore);

7) R²⁹ (wherein R²⁹ is as defined hereinbefore);

8) X¹³C₁₋₄alkylR²⁸ (wherein X¹³ and R²⁸ are as defined hereinbefore); and

9) R⁵⁴(C₁₋₄alkyl)_{q}(X⁹)ᵣR⁵⁵ (wherein q, r, X⁹, R⁵⁴ and R⁵⁵ are as defined hereinbefore); may be prepared by reacting a compound of the formula IX:

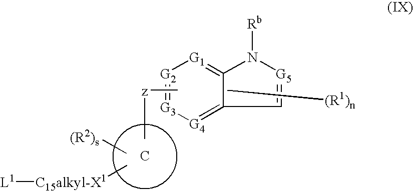

(wherein ring C, L¹, X¹, R^{b}, R¹, R², G₁, G₂, G₃, G₄, G₅, Z, n and s are as hereinbefore defined) with a compound of the formula X:

R⁶²—H (X)

(wherein R⁶² is as defined hereinbefore) to give a compound of the formula I or salt thereof. The reaction may conveniently be effected in the presence of a base (as defined hereinbefore in process (a)) and advantageously in the presence of an inert solvent or diluent (as defined hereinbefore in process (a)), and at a temperature in the range, for example 0 to 150° C., conveniently at about 50° C.

Processes (a) and (b) are preferred over processes (c) and (d).

Process (a) is preferred over processes (b), (c) and (d). (e) The production of those compounds of the formula I and salts thereof wherein one or more of the substituents (R²)ₘ is represented by —NR⁷⁶R⁷⁷, where one (and the other is hydrogen) or both of R⁷⁶ and R⁷⁷ are C₁₋₃alkyl, may be effected by the reaction of compounds of formula I wherein the substituent (R²)ₘ is an amino group and an alkylating agent, preferably in the presence of a base as defined hereinbefore. Such alkylating agents are C₁₋₃alkyl moieties bearing a displaceable moiety as defined hereinbefore such as C₁₋₃alkyl halides for example C₁₋₃alkyl chloride, bromide or iodide. The reaction is preferably effected in the presence of an inert solvent or diluent (as defined hereinbefore in process (a)) and at a temperature in the range, for example, 10 to 100° C., conveniently at about ambient temperature. The production of compounds of formula I and salts thereof wherein one or more of the substituents R² is an amino group may be effected by the reduction of a corresponding compound of formula I wherein the substituent(s) at the corresponding position(s) of ring C is/are a nitro group(s). The reduction of the nitro group may conveniently be effected by any of the procedures known for such a transformation. The reduction may be carried out, for example, by the hydrogenation of a solution of the nitro compound in the presence of an inert solvent or diluent as defined hereinbefore in the presence of a metal effective to catalyse hydrogenation reactions such as palladium or platinum. A further reducing agent is, for example, an activated metal such as activated iron (produced for example by washing iron powder with a dilute solution of an acid such as hydrochloric acid). Thus, for example, the reduction may be effected by heating the nitro compound and the activated metal in the presence of a solvent or diluent such as a mixture of water and alcohol, for example methanol or ethanol, to a temperature in the range, for example 50 to 150° C., conveniently at about 70° C.

Where the reduction is effected in the presence of activated iron, this is advantageously produced in situ, conveniently by the use of iron, generally iron powder, in the presence of acetic acid/water and preferably at about 100° C. The production of a compound of formula I and salts thereof wherein the substituent(s) at the corresponding position(s) of ring C is/are a nitro group(s) may be effected by the processes described hereinbefore and hereinafter in processes (a-d) and (i-v) using a compound selected from the compounds of the formulae (I-XVII) in which the substituent(s) at the corresponding position(s) of ring C is/are a nitro group(s).

(f) Compounds of the formula I and salts thereof wherein X¹ is —SO— or —SO₂— may be prepared by oxidation from the corresponding compound in which X¹ is —S— or —SO— (when X¹ is —SO₂— is required in the final product). Conventional oxidation conditions and reagents for such reactions are well known to the skilled chemist.

Synthesis of Intermediates

(i) The compounds of formula III and salts thereof in which L¹ is halogeno may for example be prepared by halogenating a compound of the formula XI:

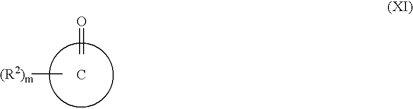

wherein ring C, R² and m are as hereinbefore defined.

Convenient halogenating agents include inorganic acid halides, for example thionyl chloride, phosphorus(III)chloride, phosphorus(V)oxychloride and phosphorus(V) chloride. The halogenation reaction may be effected in the presence of an inert solvent or diluent such as for example a halogenated solvent such as methylene chloride, trichloromethane or carbon tetrachloride, or an aromatic hydrocarbon solvent such as benzene or toluene, or the reaction may be effected without the presence of a solvent. The reaction is conveniently effected at a temperature in the range, for example 10 to 150° C., preferably in the range 40 to 100° C.

The compounds of formula XI and salts thereof may, for example, be prepared by reacting a compound of the formula XII:

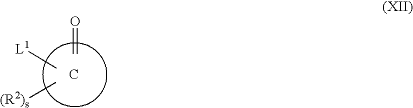

(wherein ring C, R², s and L¹ are as hereinbefore defined) with a compound of the formula VIII as hereinbefore defined. The reaction may conveniently be effected in the presence of a base (as defined hereinbefore in process (a)) and advantageously in the presence of an inert solvent or diluent (as defined hereinbefore in process (a)), advantageously at a temperature in the range, for example 10 to 150° C., conveniently at about 110° C.

The compounds of formula XI and XII and salts thereof may be prepared by any of the methods known in the art of heterocyclic organic chemistry.

The compounds of formula III and salts thereof wherein at least one R² is R⁵X¹ and wherein X¹ is —O—, —S—, —SO₂—, —OC(O)—, —C(O)NR⁷—, —SO₂NR⁸— or —NR¹⁰— (wherein R⁷, R⁸ and R¹⁰ each independently represents hydrogen, C₁₋₃alkyl or C₁₋₃alkoxyC₂₋₃alkyl), may also be prepared for example by reacting a compound of the formula XIII:

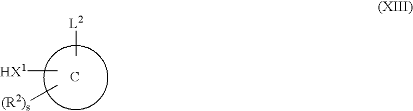

(wherein ring C, R² and s are as hereinbefore defined, X¹ is as hereinbefore defined in this section and L² represents a displaceable protecting moiety) with a compound of the formula VI as hereinbefore defined, whereby to obtain a compound of formula III in which L¹ is represented by L².

A compound of formula XIII is conveniently used in which L² represents a chloro group or a phenoxy group which may if desired carry up to 5 substituents, preferably up to 2 substituents, selected from halogeno, nitro and cyano. The reaction may be conveniently effected under conditions as described for process (b) hereinbefore.

The compounds of formula XII and salts thereof may for example be prepared by deprotecting a compound of the formula XIV:

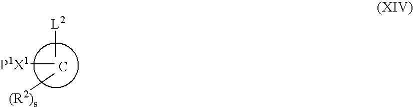

(wherein ring C, R², s and L² are as hereinbefore defined, P¹ is a protecting group and X¹ is as hereinbefore defined in the section describing compounds of the formula XIII). The choice of protecting group P¹ is within the standard knowledge of an organic chemist, for example those included in standard texts such as “Protective Groups in Organic Synthesis” T. W. Greene and R. G. M. Wuts, 2nd Ed. Wiley 1991, including N-sulphonyl derivatives (for example, p-toluenesulphonyl), carbamates (for example, t-butyl carbonyl), N-alkyl derivatives (for example, 2-chloroethyl, benzyl) and amino acetal derivatives (for example benzyloxymethyl). The removal of such a protecting group may be effected by any of the procedures known for such a transformation, including those reaction conditions indicated in standard texts such as that indicated hereinbefore, or by a related procedure. Deprotection may be effected by techniques well known in the literature, for example Where P¹ represents a benzyl group deprotection may be effected by hydrogenolysis or by treatment with trifluoroacetic acid.

One compound of formula III may if desired be converted into another compound of formula III in which the moiety L¹ is different. Thus for example a compound of formula II in which L¹ is other than halogeno, for example optionally substituted phenoxy, may be converted to a compound of formula III in which L¹ is halogeno by hydrolysis of a compound of formula III (in which L¹ is other than halogeno) to yield a compound of formula XI as hereinbefore defined, followed by introduction of halide to the compound of formula XI, thus obtained as hereinbefore defined, to yield a compound of formula III in which L¹ represents halogen.

(ii) Compounds of formula IV may be prepared by any of the methods known in the art, such as for example those described in “Indoles Part I”, “Indoles Part II”, 1972 John Wiley & Sons Ltd and “Indoles Part III” 1979, John Wiley & Sons Ltd, edited by W. J. Houlihan. Compounds of formula IV may be prepared by any of the methods described in the Examples hereinafter.

Compounds of formula IV may be prepared by any of the processes described in International Patent Application Publication No. WO 00/47212, the entire content of which is included herein by reference, with particular reference to the processes described in WO 00/47212 in Examples 48, 182 237, 242,250 and 291 therein.

For example the azaindole 2-methyl-1H-pyrrolo[2,3-b]pyridin-5-ol, may be prepared according to the method described in Reference Example 1 hereinafter.

(iii) Compounds of formula V as hereinbefore defined and salts thereof may be made by deprotecting the compound of formula XV:

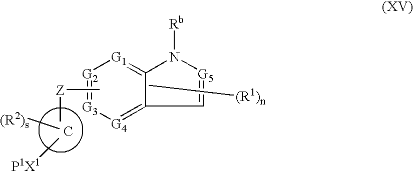

(wherein ring C, R^{b}, Z, G₁, G₂, G₃, G₄, G₅, R¹, R², P¹, n and s are as hereinbefore defined and X¹ is as hereinbefore defined in the section describing compounds of the formula V) by a process for example as described in (i) above.

Compounds of the formula XV and salts thereof may be made by reacting compounds of the formulae XIV and IV as hereinbefore defined, under the conditions described in (a) hereinbefore, to give a compound of the formula XV or salt thereof.

(iv) Compounds of the formula VII and salts thereof may be made by reacting a compound of the formula XVI:

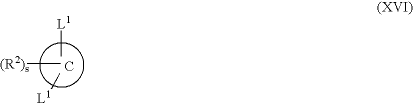

(wherein ring C, R², s and each L¹ are as hereinbefore defined and the L¹ in the 4-position and the other L¹ in a further position on ring C may be the same or different) with a compound of the formula IV as hereinbefore defined, the reaction for example being effected by a process as described in (a) above.

(v) Compounds of formula IX as defined hereinbefore and salts thereof may for example be made by the reaction of compounds of formula V as defined hereinbefore with compounds of the formula XVII:

L¹-C₁₋₅alkyl-L¹ (XVII)

(wherein L¹ is as hereinbefore defined) to give compounds of formula IX or salts thereof. The reaction may be effected for example by a process as described in (b) above.

(vi) Intermediate compounds wherein XI is —SO— or —SO₂— may be prepared by oxidation from the corresponding compound in which X¹ is —S— or —SO— (when X¹ is —SO₂— is required in the final product). Conventional oxidation conditions and reagents for such reactions are well known to the skilled chemist.

When a pharmaceutically acceptable salt of a compound of the formula I is required, it may be obtained, for example, by reaction of said compound with, for example, an acid using a conventional procedure, the acid having a pharmaceutically acceptable anion.

Many of the intermediates defined herein, for example, those of the formulae IV, V, VII, IX and XV are novel and these are provided as a further feature of the invention. The preparation of these compounds is as described herein and/or is by methods well known to persons skilled in the art of organic chemistry.

The identification of compounds which potently inhibit the tyrosine kinase activity associated with VEGF receptors such as Flt and/or KDR and which inhibit angiogenesis and/or increased vascular permeability is desirable and is the subject of the present invention. These properties may be assessed, for example, using one or more of the procedures set out below:

(a) In Vitro Receptor Tyrosine Kinase Inhibition Test

This assay determines the ability of a test compound to inhibit tyrosine kinase activity. DNA encoding VEGF, FGF or EGF receptor cytoplasmic domains may be obtained by total gene synthesis (Edwards M, International Biotechnology Lab 5(3), 19-25, 1987) or by cloning. These may then be expressed in a suitable expression system to obtain polypeptide with tyrosine kinase activity. For example VEGF, FGF and EGF receptor cytoplasmic domains, which were obtained by expression of recombinant protein in insect cells, were found to display intrinsic tyrosine kinase activity. In the case of the VEGF receptor Flt (Genbank accession number X51602), a 1.7 kb DNA fragment encoding most of the cytoplasmic domain, commencing with methiouine 783 and including the termination codon, described by Shibuya et al (Oncogene, 1990, 5: 519-524), was isolated from cDNA and cloned into a baculovirus transplacement vector (for example pAcYM1 (see The Baculovirus Expression System: A Laboratory Guide, L. A. King and R. D. Possee, Chapman and Hall, 1992) or pAc360 or pBlueBacHis (available from Invitrogen Corporation)). This recombinant construct was co-transfected into insect cells (for example Spodoptera frugiperda 21(Sf21)) with viral DNA (eg Pharmingen BaculoGold) to prepare recombinant baculovirus. (Details of the methods for the assembly of recombinant DNA molecules and the preparation and use of recombinant baculovirus can be found in standard texts for example Sambrook et al, 1989, Molecular cloning—A Laboratory Manual, 2nd edition, Cold Spring Harbour Laboratory Press and O'Reilly et al, 1992, Baculovirus Expression Vectors—A Laboratory Manual, W. H. Freeman and Co, New York). For other tyrosine kinases for use in assays, cytoplasmic fragments starting from methionine 806 (KDR, Genbank accession number L04947), methionine 668 (EGF receptor, Genbank accession number X00588) and methionine 399 (FGF R1 receptor, Genbank accession number X51803) may be cloned and expressed in a similar manner.

For expression of cFlt tyrosine kinase activity, Sf21 cells were infected with plaque-pure cFlt recombinant virus at a multiplicity of infection of 3 and harvested 48 hours later. Harvested cells were washed with ice cold phosphate buffered saline solution (PBS) (10 nM sodium phosphate pH 7.4, 138 nM sodium chloride, 2.7 mM potassium chloride) then resuspended in ice cold HNTG/PMSF (20 mM Hepes pH 7.5, 150 mM sodium chloride, 10% v/v glycerol, 1% v/v Triton X100, 1.5 mM magnesium chloride, 1 mM ethylene glycol-bis(βaminoethyl ether) N,N,N′,N′-tetraacetic acid (EGTA), 1 mM PMSF (phenylmethylsulphonyl fluoride); the PMSF is added just before use from a freshly-prepared 100 nM solution in methanol) using 1 ml HNTG/PMSF per 10 million cells. The suspension was centrifuged for 10 minutes at 13,000 rpm at 4° C., the supernatant (enzyme stock) was removed and stored in aliquots at −70° C. Each new batch of stock enzyme was titrated in the assay by dilution with enzyme diluent (100 nm Hepes pH 7.4, 0.2 mM sodium orthovanadate, 0.1% v/v Triton X100, 0.2 nM dithiothreitol). For a typical batch, stock enzyme is diluted 1 in 2000 with enzyme diluent and 50,1i of dilute enzyme is used for each assay well.

A stock of substrate solution was prepared from a random copolymer containing tyrosine, for example Poly (Glu, Ala, Tyr) 6:3:1 (Sigma P3899), stored as 1 mg/ml stock in PBS at −20° C. and diluted 1 in 500 with PBS for plate coating.

On the day before the assay 100 μl of diluted substrate solution was dispensed into all wells of assay plates (Nunc maxisorp 96-well immunoplates) which were sealed and left overnight at 4° C.

On the day of the assay the substrate solution was discarded and the assay plate wells were washed once with PBST (PBS containing 0.05% v/v Tween 20) and once with 500 nM Hepes pH 7.⁴.

Test compounds were diluted with 10% dimethylsulphoxide (DMSO) and 25 μl of diluted compound was transferred to wells in the washed assay plates. “Total” control wells contained 10% DMSO instead of compound. Twenty five microlitres of 40 mM manganese(II)chloride containing 8 μM adenosine-5′-triphosphate (ATP) was added to all test wells except “blank” control wells which contained manganese(II)chloride without ATP. To start the reactions 50 μl of freshly diluted enzyme was added to each well and the plates were incubated at room temperature for 20 minutes. The liquid was then discarded and the wells were washed twice with PBST. One hundred microlitres of mouse IgG anti-phosphotyrosine antibody (Upstate Biotechnology Inc. product 05-321), diluted 1 in 6000 with PBST containing 0.5% w/v bovine serum albumin (BSA), was added to each well and the plates were incubated for 1 hour at room temperature before discarding the liquid and washing the wells twice with PBST. One hundred microlitres of horse radish peroxidase (HRP)-linked sheep anti-mouse Ig antibody (Amersham product NXA 931), diluted 1 in 500 with PBST containing 0.5% w/v BSA, was added and the plates were incubated for 1 hour at room temperature before discarding the liquid and washing the wells twice with PBST. One hundred microlitres of 2,2′-azino-bis(3-ethylbenzthiazoline-6-sulphonic acid) (ABTS) solution, freshly prepared using one 50 mg ABTS tablet (Boehringer 1204 521) in 50 ml freshly prepared 50 nM phosphate-citrate buffer pH 5.0+0.03% sodium perborate (made with 1 phosphate citrate buffer with sodium perborate (PCSB) capsule (Sigma P4922) per 100 ml distilled water), was added to each well. Plates were then incubated for 20-60 minutes at room temperature until the optical density value of the “total” control wells, measured at 405 nm using a plate reading spectrophotometer, was approximately 1.0. “Blank” (no ATP) and “total” (no compound) control values were used to determine the dilution range of test compound which gave 50% inhibtion of enzyme activity.

(b) In Vitro HUVEC Proliferation Assay

This assay determines the ability of a test compound to inhibit the growth factor-stimulated proliferation of human umbilical vein endothelial cells (HUVEC).

HUVEC cells were isolated in MCDB 131 (Gibco BRL)+7.5% v/v foetal calf serum (FCS) and were plated out (at passage 2 to 8), in MCDB 131+2% v/v FCS+3 μg/ml heparin+1 μg/ml hydrocortisone, at a concentration of 1000 cells/well in 96 well plates. After a minimum of 4 hours they were dosed with the appropriate growth factor (i.e. VEGF 3 ng/ml, EGF 3 ng/ml or b-FGF 0.3 ng/ml) and compound. The cultures were then incubated for 4 days at 37° C. with 7.5% CO₂. On day 4 the cultures were pulsed with 1 μCi/well of tritiated-thymidine (Amersham product TRA 61) and incubated for 4 hours. The cells were harvested using a 96-well plate harvester (Tomtek) and then assayed for incorporation of tritium with a Beta plate counter. Incorporation of radioactivity into cells, expressed as cpm, was used to measure inhibition of growth factor-stimulated cell proliferation by compounds.

(c) In Vivo Solid Tumour Disease Model

This test measures the capacity of compounds to inhibit solid tumour growth.

CaLu-6 tumour xenografts were established in the flank of female athymic Swiss nu/nu mice, by subcutaneous injection of 1×10⁶ CaLu-6 cells/mouse in 100 μl of a 50% (v/v) solution of Matrigel in serum free culture medium. Ten days after cellular implant, mice were allocated to groups of 8-10, so as to achieve comparable group mean volumes. Tumours were measured using vernier calipers and volumes were calculated as: (l×w)×√(l×w)×(π/6), where l is the longest diameter and w the diameter perpendicular to the longest. Test compounds were administered orally once daily for a minimum of 21 days, and control animals received compound diluent. Tumours were measured twice weekly. The level of growth inhibition was calculated by comparison of the mean tumour volume of the control group versus the treatment group using a Student T test and/or a Mann-Whitney Rank Sum Test. The inhibitory effect of compound treatment was considered significant when p<0.05.

According to a further aspect of the invention there is provided a pharmaceutical composition which comprises a compound of the formula I as defined hereinbefore or a pharmaceutically acceptable salt thereof, in association with a pharmaceutically acceptable excipient or carrier.

The composition may be in a form suitable for oral administration, for example as a tablet or capsule, for parenteral injection (including intravenous, subcutaneous, intramuscular, intravascular or infusion) for example as a sterile solution, suspension or emulsion, for topical administration for example as an ointment or cream or for rectal administration for example as a suppository. In general the above compositions may be prepared in a conventional manner using conventional excipients.

The compositions of the present invention are advantageously presented in unit dosage form. The compound will normally be administered to a warm-blooded animal at a unit dose within the range 5-5000 ng per square metre body area of the animal, i.e. approximately 0.1-100 mg/l(g. A unit dose in the range, for example, l-100 mg/kg, preferably 1-50 mg/kg is envisaged and this normally provides a therapeutically-effective dose. A unit dose form such as a tablet or capsule will usually contain, for example 1-250 mg of active ingredient.

According to a further aspect of the present invention there is provided a compound of the formula I or a pharmaceutically acceptable salt thereof as defined hereinbefore for use in a method of treatment of the human or animal body by therapy.

We have found that compounds of the present invention inhibit VEGF receptor tyrosine kinase activity and are therefore of interest for their antiangiogenic effects and/or their ability to cause a reduction in vascular permeability.

A further feature of the present invention is a compound of formula I, or a pharmaceutically acceptable salt thereof, for use as a medicament, conveniently a compound of formula I, or a pharmaceutically acceptable salt thereof, for use as a medicament for producing an antiangiogenic and/or vascular permeability reducing effect in a warm-blooded animal such as a human being.

Thus according to a further aspect of the invention there is provided the use of a compound of the formula I, or a pharmaceutically acceptable salt thereof in the manufacture of a medicament for use in the production of an antiangiogenic and/or vascular permeability reducing effect in a warm-blooded animal such as a human being.

According to a further feature of the invention there is provided a method for producing an antiangiogenic and/or vascular permeability reducing effect in a warm-blooded animal, such as a human being, in need of such treatment which comprises administering to said animal an effective amount of a compound of formula I or a pharmaceutically acceptable salt thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

The antiangiogenic and/or vascular permeability reducing treatment defined hereinbefore may be applied as a sole therapy or may involve, in addition to a compound of the invention, one or more other substances and/or treatments. Such conjoint treatment may be achieved by way of the simultaneous, sequential or separate administration of the individual components of the treatment. In the field of medical oncology it is normal practice to use a combination of different forms of treatment to treat each patient with cancer. In medical oncology the other component(s) of such conjoint treatment in addition to the antiangiogenic and/or vascular permeability reducing treatment defined hereinbefore may be: surgery, radiotherapy or chemotherapy. Such chemotherapy may cover three main categories of therapeutic agent:

(i) other antiangiogenic agents that work by different mechanisms from those defined hereinbefore (for example linomide, inhibitors of integrin αvβ3 function, angiostatin, razoxin, thalidomide), and including vascular targeting agents (for example combretastatin phosphate and the vascular damaging agents described in International Patent Application Publication No. WO 99/02166 the entire disclosure of which document is incorporated herein by reference, (for example N-acetylcolchinol-O-phosphate), and in International Patent Application Publication No. WO 00/40529 the entire disclosure of which document is incorporated herein by reference);

(ii) cytostatic agents such as antioestrogens (for example tamoxifen, toremifene, raloxifene, droloxifene, iodoxyfene), progestogens (for example megestrol acetate), aromatase inhibitors (for example anastrozole, letrazole, vorazole, exemestane), antiprogestogens, antiandrogens (for example flutamide, nilutamide, bicalutamide, cyproterone acetate), LHRH agonists and antagonists (for example goserelin acetate, luprolide), inhibitors of testosterone 5α-dihydroreductase (for example finasteride), anti-invasion agents (for example metalloproteinase inhibitors like marimastat and inhibitors of urokinase plasminogen activator receptor function) and inhibitors of growth factor function, (such growth factors include for example platelet derived growth factor and hepatocyte growth factor such inhibitors include growth factor antibodies, growth factor receptor antibodies, tyrosine kinase inhibitors and serine/threonine kinase inhibitors); and

(iii) antiproliferative/antineoplastic drugs and combinations thereof, as used in medical oncology, such as antimetabolites (for example antifolates like methotrexate, fluoropyrimidines like 5-fluorouracil, purine and adenosine analogues, cytosine arabinoside); antitumour antibiotics (for example anthracyclines like doxorubicin, daunomycin, epirubicin and idalubicin, mitomycin-C, dactinomycin, mithramiycin); platinum derivatives (for example cisplatin, carboplatin); alkylating agents (for example nitrogen mustard, melphalan, chlorambucil, busulphan, cyclophosphamide, ifosfamide, nitrosoureas, thiotepa); antimitotic agents (for example vinca alkaloids like vincristine and taxoids like taxol, taxotere); topoisomerase inhibitors (for example epipodophyllotoxins like etoposide and teniposide, amsacrine, topotecan, and also irinotecan); also enzymes (for example asparaginase); and thymidylate synthase inhibitors (for example raltitrexed);

and additional types of chemotherapeutic agent include:

(iv) biological response modifiers (for example interferon); and

(v) antibodies (for example edrecolomab).

For example such conjoint treatment may be achieved by way of the simultaneous, sequential or separate administration of a compound of formula I as defined hereinbefore, and a vascular targeting agent described in WO 99/02166 such as N-acetylcolchinol-O-phosphate (Exampe 1 of WO 99/02166).

As stated above the compounds defined in the present invention are of interest for their antiangiogenic and/or vascular permeability reducing effects. Such compounds of the invention are expected to be useful in a wide range of disease states including cancer, diabetes, psoriasis, rheumatoid arthritis, Kaposi's sarcoma, haemangioma, acute and chronic nephropathies, atheroma, arterial restenosis, autoimmune diseases, acute inflammation, excessive scar formation and adhesions, lymphoedema, endometriosis, dysfunctional uterine bleeding and ocular diseases with retinal vessel proliferation. In particular such compounds of the invention are expected to slow advantageously the growth of primary and recurrent solid tumours of, for example, the colon, breast, prostate, lungs and skin. More particularly such compounds of the invention are expected to inhibit the growth of those primary and recurrent solid tumours which are associated with VEGF, especially those tumours which are significantly dependent on VEGF for their growth and spread, including for example, certain tumours of the colon, breast, prostate, lung, vulva and skin.

In addition to their use in therapeutic medicine, the compounds of formula I and their pharmaceutically acceptable salts are also useful as pharmacological tools in the development and standardisation of in vitro and in vivo test systems for the evaluation of the effects of inhibitors of VEGF receptor tyrosine kinase activity in laboratory animals such as cats, dogs, rabbits, monkeys, rats and mice, as part of the search for new therapeutic agents.

It is to be understood that where the term “ether” is used anywhere in this specification it refers to diethyl ether.

The invention will now be illustrated in the following non-limiting Examples in which, unless otherwise stated:—

(i) evaporations were carried out by rotary evaporation in vacuo and work-up procedures were carried out after removal of residual solids such as drying agents by filtration;

(ii) operations were carried out at ambient temperature, that is in the range 18-25° C. and under an atmosphere of an inert gas such as argon;

(iii) column chromatography (by the flash procedure) and medium pressure liquid chromatography (MPLC) were performed on Merck Kieselgel silica (Art. 9385) or Merck Lichroprep RP-18 (Art. 9303) reversed-phase silica obtained from E. Merck, Darnstadt, Germany;

(iv) yields are given for illustration only and are not necessarily the maximum attainable;

(v) melting points are uncorrected and were determined using a Mettler SP62 automatic melting point apparatus, an oil-bath apparatus or a Koffler hot plate apparatus.

(vi) the structures of the end-products of the formula I were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

(vii) intermediates were not generally fully characterised and purity was assessed by thin layer chromatography (TLC), high-performance liquid chromatography (HPLC), infra-red (IR) or NMR analysis;

(viii) HPLC were run under 2 different conditions:

1) on a TSK Gel super ODS 2 μM 4.6 mm×5 cm column, eluting with a gradient of methanol in water (containing 1% acetic acid) 20 to 100% in 5 minutes. Flow rate 1.4 ml/minute. Detection: U.V. at 254 nm and light scattering detections;

2) on a TSK Gel super ODS 2 μM 4.6 mm×5 cm column, eluting with a gradient of methanol in water (containing 1% acetic acid) 0 to 100% in 7 minutes. Flow rate 1.4 ml/minute. Detection: U.V. at 254 nm and light scattering detections.

(ix) petroleum ether refers to that fraction boiling between 40-60° C.

(x) the following abbreviations have been used:

DMF N,N-dimethylformamide

DMSO dimethylsulphoxide

TFA trifluoroacetic acid

NMP 1-methyl-2-pyrrolidinone

THF tetrahydrofuran

HMDS 1,1,1,3,3,3-hexamethyldisilazane.

HPLC RT HPLC retention time

DEAD diethyl azodicarboxylate

DMA dimethxlacetamide

DMAP 4-dimethylaminopyridine

### EXAMPLE 1

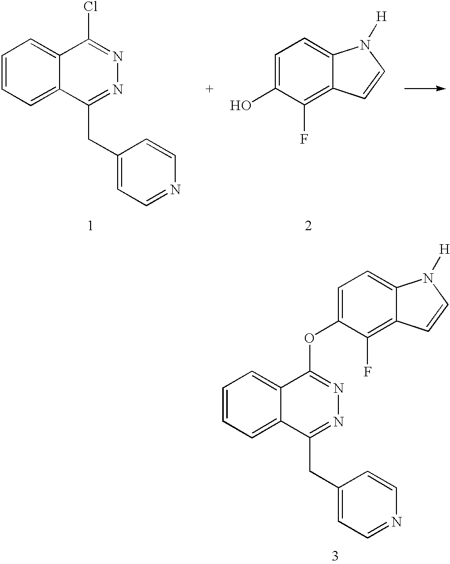

Under nitrogen a suspension of 1-chloro-4-(4-pyridylmethyl)phthalazine (150 mg, 0.58 mmol), (J. Med. Chem. 2000, 43, 2310-2323), 4-fluoro-5-hydroxyindole (106 mg, 0.7 mmol) and cesium carbonate (306 mg, 0.938 mmol) in DMF (5.5 ml) was heated at 95° C. for 2 hours. The volatiles were removed under vacuum and the residue was purified by column chromatography eluting with methylene chloride followed by methylene chloride/ethyl acetate/methanol (45/50/5 followed by 40/50/10). The fractions containing the expected product were combined and evaporated to give 1-(4-fluoroindol-5-yloxy)-4-(4-pyridylmethyl)phthalazine (57 mg, 22%).

¹H NMR Spectrum: (DMSOd₆) 4.66 (s, 2H); 6.57 (s, 1H); 7.15 (dd, 1H); 7.35 (m, 3H); 7.5 (m, 1H); 8.12 (m, 2H); 8.3 (m, 1H); 8.48 (d, 2H); 8.5 (in, 1H) MS-ESI: 371.6 [MH]⁺

The starting material was prepared as follows:

A mixture of 2-fluoro-4-nitrophenol (15 g, 95.5 mmol) and benzyl bromide (18 g, 105 mmol) in acetone (125 ml) containing potassium carbonate (26.5 g, 190 mmol) was heated at reflux for 2 hours. The volatiles were removed and the residue was partitioned between 2N hydrochloric acid and ethyl acetate. The organic layer was separated, washed with water, brine, dried (MgSO₄) and the volatiles were removed under vacuum. The solid was triturated with petroleum ether to give 2-fluoro-4-nitro-benzyloxybenzene (23 g, 97%).

¹H NMR Spectrum: (CDCl₃) 5.3 (s, 2); 7.1 (t, 1H); 7.35-7.55 (m, 5); 8.0(m, 2H)

To a solution of potassium tert-butoxide (1.72 g, 15.4 mmol) in DMF (15 ml) cooled at −30° C., was added dropwise a solution of 2-fluoro-4-nitro-benzyloxybenzene (1.73 g, 7 mmol) and 4-chlorophenoxyacetonitrile (1.29 g, 7.7 mmol) while maintaining the temperature below −25° C. After completion of addition, the mixture was stirred for 30 minutes at −20° C. and then poured onto a mixture of cold 1N hydrochloric acid and ether. The organic layer was separated, washed with 1N sodium hydroxide, followed by water, brine and dried (MgSO₄). The volatiles were removed under vacuum and the residue was purified by column chromatography eluting with methylene chloride/petroleum ether (3/1) to give a mixture of 3-cyanomethyl-2-fluoro-4-nitrobenzyloxybenzene and 5-cyanomethyl-2-fluoro-4-nitrobenzyloxybenzene (1.2 g, 60%).

¹H NMR Spectrum: (DMSOd₆) 4.22 (s, 2H, 3-cyanomethyl isomer); 4.3 (s, 2H, 5-cyanomethyl isomer); 5.32 (s, 2H, 5-cyanomethyl isomer); 5.36 (s, 2H, 3-cyanomethyl isomer); 7.3-7.7 (m, 6H); 8.1 (d, 1H, 3-cyanomethyl isomer); 8.2 (d, 1H, 5-cyanomethyl isomer)

A solution of a mixture of 3-cyanomethyl-2-fluoro-4-nitrobenzyloxybenzene and 5-cyanomethyl-2-fluoro-4-nitrobenzyloxybenzene (23 g, 80.4 mmol) in ethanol (220 ml) and acetic acid (30 ml) containing 10% palladium on charcoal (600 mg) was hydrogenated under 3 atmospheres pressure until hydrogen uptake ceased. The mixture was filtered and the filtrate was evaporated under vacuum. The residue was purified on column chromatography using a Prochrorn® equipment eluting with methylene chloride/petroleum ether (20/80) to give 4-fluoro-5-hydroxyindole (2.48 g) and 6-fluoro-5-hydroxyindole (3.5 g).

4-fluoro-5-hydroxyindole:

¹H NMR Spectrum: (DMSOd₆) 6.32 (s, 1H); 6.75 (dd, 1H); 7.0 (d, 1H); 7.28 (dd, 1H); 8.8 (br s, 1H); 11.05 (br s, 1H)

6-fluoro-5-hydroxyindole:

¹H NMR Spectrum: (DMSOd₆) 6.25 (s, 1H); 7.0 (d, 1H); 7.12 (d, 1H); 7.2 (dd, 1H); 9.0 (br s, 1H)

### EXAMPLES 2-8

Using an analogous procedure to that described in Example 1, 1-chloro-4-(4-pyridylmethyl)phthalazine (150 mg) was reacted with the appropriate hydroxyindole (0.7 mmol) to give the corresponding compounds described in Table I:

TABLE I

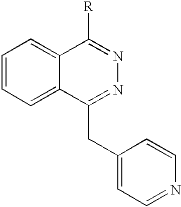

| Ex- | Weight | Yield | MS-ESI | | |
|---|---|---|---|---|---|
| ample | (mg) | (%) | [MH]⁺ | R | Note |
| 2 | 75 | 29 | 371.6 | 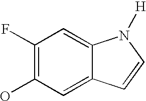 | a |
| 3 | 106 | 41 | 367.6 | 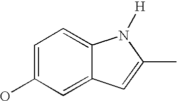 | b |
| 4 | 73 | 29 | 353.6 | 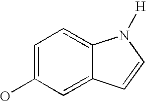 | c |
| 5 | 86 | 35 | 353.6 | 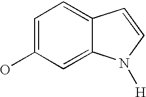 | d |
| 6 | 110 | 43 | 367.6 | 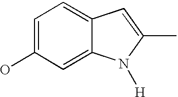 | e |
| 7 | 88 | 33 | 381.6 | 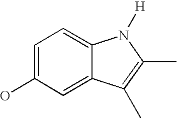 | f |
| 8 | 89 | 33 | 381.6 | 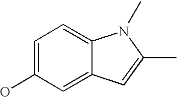 | g |

a 1-Chloro-4-(4-pyridylmethyl)phthalazine (150 mg) was reacted with 6-fluoro-5-hydroxyindole (106 mg, 0.7 mmol), (prepared as described for the starting material in Example 1), to give 1-(6-fluoroindol-5-yloxy)-4-(4-pyridylmethyl)phthalazine. ¹H NMR Spectrum: (DMSOd₆) 4.65(s, 2H); 6.5(s, 2H); 7.32(d, 2H); 7.4(d, 1H); 7.45(s, 1H); 7.62(d, 1H); 8.1(m, 2H); 8.3(m, 1H); 8.48(d, 2H); 8.5(m, 1H)
b 1-Chloro-4-(4-pyridylmethyl)phthalazine (150 mg) was reacted with 5-hydroxy-2-methylindole (104 mg, 0.7 mmol) to give 1-(2-methylindol-5-yloxy)-4-(4-pyridylmethyl)phthalazine. ¹H NMR Spectrum: (DMSOd₆) 2.42(s, 3H); 4.65(s, 2H); 6.18(s, 1H); 6.95(dd, 1H); 7.32(m, 4H); 8.1(m, 2H); 8.25(m,1H); 8.48(m, 3H)
c 1-Chloro-4-(4-pyridylmethyl)phthalazine (150 mg) was reacted with 5-hydroxyindole (94 mg, 0.7 mmol) to give 1-(indol-5-yloxy)-4-(4-pyridylmethyl)phthalazine. ¹H NMR Spectrum: (DMSOd₆) 4.65(s, 2H); 6.48(s, 1H); 7.05(dd, 1H); 7.32(d, 2H); 7.4-7.5(m, 3H); 8.07(m, 2H); 8.25(m, 1H); 8.4-8.5(m, 3H)
d 1-Chloro-4-(4-pyridylmethyl)phthalazine (150 mg) was reacted with 6-hydroxyindole (94 mg, 0.7 mmol) to give 1-(indol-6-yloxy)-4-(4-pyridylmethyl)phthalazine. ¹H NMR Spectrum: (DMSOd₆) 4.65(s, 2H); 6.5(s, 1H); 6.98(dd, 1H); 7.35(d, 2H); 7.37(s, 1H); 7.4(m, 1H); 7.6(d, 1H); 8.1(m, 2H); 8.28(m, 1H); 8.4-8.5(m, 3H)
e 1-Chloro-4-(4-pyridylmethyl)phthalazine (150 mg) was reacted with 6-hydroxy-2-methylindole (104 mg, 0.7 mmol), (Eur. J. Med. Chem. 1975, 10, 187), to give 1-(2-methylindol-6-yloxy)-4-(4-pyridylmethyl)phthalazine. ¹H NMR Spectrum: (DMSOd₆) 2.41(s, 3H); 4.65(s, 2H); 6.18(s, 1H); 6.9(dd, 1H); 7.21(s, 1H); 7.32(d, 2H); 7.45(d, 1H); 8.05-8.15(m, 2H); 8.25(m, 1H); 8.4-8.5(m, 3H)
f 1-Chloro-4-(4-pyridylmethyl)phthalazine (150 mg) was reacted with 2,3-dimethyl-5-hydroxyindole (113 mg, 0.7 mmol), (Arch. Pharm. 1972, 305, 159), to give 1-(2,3-dimethylindol-5-yloxy)-4-(4-pyridylmethyl)phthalazine. ¹H NMR Spectrum: (DMSOd₆) 2.15 (s, 3H); 2.35(s, 3H); 4.65(s, 2H); 6.93(dd, 1H); 7.2-7.4(m, 4H); 8.0-8.12(m, 2H); 8.25(m, 1H); 8.4-8.5(m, 3H)
g 1-Chloro-4-(4-pyridylmethyl)phthalazine (150 mg) was reacted with 1,2-dimethyl-5-hydroxyindole (113 mg, 0.7 mmol), (Tetrahedron, 1994, 50, 13433), to give 1-(1,2-dimethylindol-5-yloxy)-4-(4-pyridylmethyl)phthalazine. ¹H NMR Spectrum: (DMSOd₆) 2.45(s, 3H); 3.72(s, 3H); 4.64(s, 2H); 6.25(s, 1H); 7.03(dd, 1H); 7.32(d, 2H); 7.35(s, 1H); 7.46(d, 1H); 8.02-8.1(m, 1H); 8.25(m, 1H); 8.46(m, 3H)

### EXAMPLE 9

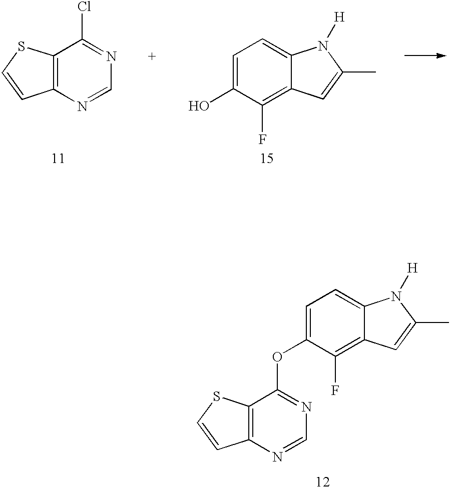

Using an analogous procedure to that described in Example 1, 4-chlorothieno[3,2-d]pyrimidine (150 mg, 0.88 mmol) was reacted with 4-fluoro-5-hydroxy-2-methylindole (174 mg, 1.05 mmol) to give 4-(4-fluoro-2-methylindol-5-yloxy)thieno[3,2-d]pyrimidine (18 mg, 7%).

¹H NMR Spectrum: (DMSOd₆) 2.41 (s, 3H); 6.25 (s, 1H); 7.03 (dd, 1H); 7.18 (d, 1H); 7.7 (d, 1H); 8.5 (d, 1H); 8.68 (s, 1H)

MS-ESI: 300 [MH]⁺

The starting material was prepared as follows:

To a suspension of sodium hydride (5.42 g, 226 mmol) (prewashed with pentane) in THF (100 ml) cooled at 10° C. was added ethyl acetoacetate (29.4 g, 226 mmol) while keeping the temperature below 15° C. After completion of addition, the mixture was further stirred for 15 minutes and cooled to 5° C. A solution of 1,2,3-trifluoro-4-nitrobenzene (20 g, 113 mmol) in THF (150 ml) was added while keeping the temperature below 5° C. The mixture was then left to warm up to ambient temperature and stirred for 24 hours. The volatiles were removed under vacuum and the residue was partitioned between ethyl acetate and 2N aqueous hydrochloric acid. The organic layer was washed with water, brine, dried (MgSO₄) and evaporated. The residue was dissolved in concentrated hydrochloric acid (650 ml) and acetic acid (600 ml) and the mixture was heated at reflux for 15 hours. After cooling, the volatiles were removed under vacuum and the residue was partitioned between aqueous sodium hydrogen carbonate (5%) and ethyl acetate. The organic layer was washed with sodium hydrogen carbonate, water, brine, dried (MgSO₄) and evaporated. The residue was purified by column chromatography eluting with ethyl acetate/petroleum ether (75/25) to give 3-acetylmethyl-1,2-difluoro-4-nitrobenzene (17.5 g, 72%).

¹H NMR Spectrum: (CDCl₃) 2.4 (s, 3H); 4.25 (s, 2H); 7.25 (dd, 1H); 8.0 (dd, 1H)

A solution of 3-acetylmethyl-1,2-difluoro-4-nitrobenzene (500 mg, 2.3 mmol) in methylene chloride (5 ml) containing moutmorillonite K10 (1 g) and trimethyl orthoformate (5 ml) was stirred for 24 hours at ambient temperature. The solid was filtered, washed with methylene chloride and the filtrate was evaporated to give 1,2-difluoro-3-(2,2-dimethoxypropyl)-4-nitrobenzene (534 mg, 88%).

¹H NMR Spectrum: (CDCl₃) 1.2 (s, 3H); 3.2 (s, 6H); 3.52 (s, 2H); 7.18 (dd, 1H); 7.6 (m, 1H)

To a solution of benzyl alcohol (221 mg, 2.05 mmol) in DMA (1.5 ml) was added 60% sodium hydride (82 mg, 2.05 mmol). The mixture was stirred for 1 hour at ambient temperature. A solution of 1,2-difluoro-3-(2,2-dimethoxypropyl)-4-nitrobenzene (534 mg, 2.05 mmol) in DMA (1.5 ml) was added and the mixture was stirred for 3 hours at ambient temperature. The mixture was diluted with 1N hydrochloric acid (10 ml) and extracted with ethyl acetate. The organic layer was evaporated and the residue was dissolved in THF (2 ml) and 6N hydrochloric acid (0.3 ml) was added. The mixture was stirred for 1 hour at ambient temperature and the solvents were removed under vacuum. The residue was partitioned between ethyl acetate and water. The organic layer was separated, washed with brine, dried (MgSO₄) and evaporated. The solid was triturated with ether, filtered, washed with ether and dried under vacuum to give 3-acetylmethyl-1-benzyloxy-2-fluoro-4-nitrobenzene (350 mg, 56%).

¹H NMR Spectrum: (CDCl₃) 2.35 (s, 3H); 4.25 (s, 2); 5.25 (s, 2H); 7.0 (dd, 1H); 7.32-7.5 (m, 5H); 8.0 (dd, 1H)

A solution of 3-acetylmethyl-1-benzyloxy-2-fluoro-4-nitrobenzene (300 mg, 0.99 mmol) in ethanol (10 ml) and acetic acid (1 ml) containing 10% palladium on charcoal (30 mg) was hydrogenated at 2 atmospheres pressure for 2 hours. The mixture was filtered and the filtrate was evaporated. The residue was dissolved in ethyl acetate and the organic layer was washed with aqueous sodium hydrogen carbonate, brine and evaporated to give 4-fluoro-5-hydroxy-2-methylindole. The residue was purified by column chromatography eluting with ethyl acetate/petroleum ether (3/7) to give 4-fluoro-5-hydroxy-2-methylindole (63 mg, 30%).

MS-ESI: 166 [MH]⁺

¹H NMR Spectrum: (DMSOd₆) 2.35 (s, 3H); 6.05 (s, 1H); 6.65 (dd, 1H); 6.9 (d, 1H); 8.75 (s, 1×); 10.9 (s, 1H)

¹³C NMR Spectrum: (DMSOd₆) 13.5; 94,0; 106,0; 112; 118.5 (d); 132 (d); 136 (d), 136.5; 142.5 (d)

Alternatively the 4-fluoro-5-hydroxy-2-methylindole may be prepared as follows:

To a solution of 2-fluoro-4-nitroanisole (9.9 g, 58 mmol) and 4-chlorophenoxyacetonitrile (10.7 g, 64 mmol) in DMF (50 ml) cooled at −15° C. was added potassium tert-butoxide (14.3 g, 127 mmol) in DMF (124 ml). After stirring for 30 minutes at −15° C., the mixture was poured onto cooled 1N hydrochloric acid. The mixture was extracted with ethyl acetate. The organic layer was washed with 1N sodium hydroxide, brine, dried (MgSO₄) and evaporated. The residue was purified by column chromatography eluting with methylene chloride. The fractions containing the expected product were combined and evaporated. The residue was dissolved in ethanol (180 ml) and acetic acid (24 ml) containing 10% palladium on charcoal (600 mg) and the mixture was hydrogenated under 3 atmospheres pressure for 2 hours. The mixture was filtered, and the volatiles were removed under vacuum. The residue was partitioned between ethyl acetate and water. The organic layer was separated, and washed with saturated sodium hydrogen carbonate followed by brine, dried (MgSO₄) and evaporated. The residue was purified by column chromatography eluting with methylene chloride to give a mixture of 4-fluoro-5-methoxyindole and 6-fluoro-5-methoxyindole (5.64 g, 59%) in a ratio 1/2.

¹H NMR Spectrum: (DMSOd₆) 3.85 (s, 3H); 6.38 (s, 1H, 6-Fluoro); 6.45 (s, 1H; 4-Fluoro); 6.9-7.4 (m, 3H)

A solution of 4-fluoro-5-methoxyindole and 6-fluoro-5-methoxyindole in a ratio 1/2 (496 mg, 3 mmol), di-tertbutyl dicarbonate (720 mg, 3.3 mmol) in acetonitrile (12 ml) containing DMAP (18 mg, 0.15 mmol) was stirred at ambient temperature for 24 hours. The volatiles were removed under vacuum. The residue was dissolved in ethyl acetate, washed with 1N hydrochloric acid, followed by water, brine, dried (MgSO₄) and evaporated to give a mixture of 4-fluoro-5-methoxy-1-tert-butoxycarbonylindole and 6-fluoro-5-methoxy-1-tert-butoxycarbonylindole in a ratio 1/2 (702 mg, 88%).

¹H NMR Spectrum: (DMSOd₆) 1.65 (s, 9H); 3.9 (s, 3H); 6.6 (d, 1H, 6-fluoro); 6.72 (d, 1H, 4-fluoro); 7.2 (t, 1H, 6-fluoro); 7.4 (d, 1H, 4-fluoro); 7.62 (d, 1H, 6-fluoro); 7.68 (d, 1H, 4-fluoro); 7.78 (s, 1H, 4-fluoro); 7.85 (s, 1H, 6-fluoro)

To a solution of 4-fluoro-5-methoxy-1-tert-butoxycarbonylindole and 6-fluoro-5-methoxy-1-tert-butoxycarbonylindole in a ratio 1/2 (8.1 g, 30.5 mmol) in THF (100 ml) cooled at −65° C. was added tert-butyllithium (1.7 M) (23 ml, 35.7 mmol). After stirring for 4 hours at −70° C., methyl iodide (8.66 g, 61 mmol) was added and the mixture was left to warm-up to ambient temperature. Water was added and the mixture was extracted with ether. The organic layer was washed with water, brine, dried (MgSO₄) and evaporated and was used directly in the next step.

The crude product was dissolved in methylene chloride (100 ml) and TFA (25 ml) was added. After stirring for 1 hour at ambient temperature, the volatiles were removed under vacuum. The residue was dissolved in ethyl acetate and the organic layer was washed with 1N sodium hydroxide, followed by water, brine, dried (MgSO₄) and evaporated. The residue was purified by column chromatography, eluting with ethyl acetate/petroleum ether (3/7) to give 6-fluoro-5-methoxy-2-methylindole (1.6 g) and 4-fluoro-5-methoxy-2-methylindole (0.8 g, 48%).

6-fluoro-5-methoxy-2-methylindole:

MS-ESI: 180 [MH]⁺

¹H NMR Spectrum: (DMSOd₆) 2.35 (s, 3H); 3.8 (s, 3H); 6.05 (s, 1H); 7.1 (s, 1H); 7.12 (s, 1H); 10.8 (s, 1H) 4-fluoro-5-methoxy-2-methylindole:

MS-ESI: 180 [MH]⁺

¹H NMR Spectrum: (DMSOd₆) 2.35 (s, 3H); 3.8 (s, 3H); 6.15 (s, 1H); 6.9 (t, 1H); 7.05 (d, 1H); 11.0 (s, 1H)

To a solution of 4-fluoro-5-methoxy-2-methylindole (709 mg, 3.95 mmol) in methylene chloride (9 ml) cooled at −30° C. was added a solution of boron tribromide (2.18 g, 8.7 mmol) in methylene chloride (1 ml). After stirring for 1 hour at ambient temperature, the mixture was poured onto water and was diluted with methylene chloride. The pH of the aqueous layer was adjusted to 6. The organic layer was separated, washed with water, brine, dried (MgSO₄) and evaporated. The residue was purified by column chromatography, eluting with ethyl acetate/petroleum ether (3/7) to give 4-fluoro-5-hydroxy-2-methylindole (461 mg, 70%).

MS-ESI: 166 [MH]⁺

¹H NMR Spectrum: (DMSOd₆) 2.35 (s, 3H); 6.05 (s, 1H); 6.65 (dd, 1H); 6.9 (d, 1H); 8.75 (s, 1H); 10.9 (s, 1H)

¹³C NMR Spectrum: (DMSOd₆) 13.5; 94,0; 106,0; 112; 118.5 (d); 132 (d); 136 (d); 136.5; 142.5 (d)

Alternatively the 4-fluoro-5-hydroxy-2-methylindole may be prepared as follows:

A solution of sodium methoxide (freshly prepared from sodium (1.71 g) and methanol (35 ml)) was added to a solution of 1,2-difluoro-3-(2,2-dimethoxypropyl)-4-nitrobenzene (16.2 g, 62 mmol), (prepared as described above), in methanol (200 ml) cooled at 5° C. The mixture was left to warm to ambient temperature and was stirred for 3 days. The volatiles were removed under vacuum and the residue was partitioned between ethyl acetate and 2N hydrochloric acid (1 ml). The organic layer was concentrated to a total volume of 100 ml and THF (100 ml) and 6N hydrochloric acid (25 ml) were added. The mixture was stirred for 1 hour at ambient temperature. The volatiles were removed under vacuum and the residue was partitioned between ethyl acetate and water. The organic layer was separated, washed with water, brine, dried (MgSO₄) and evaporated. The residue was purified by column chromatography eluting with ethyl acetate/petroleum ether (3/7) to give 3-acetylmethyl-2-fluoro-1-methoxy-4-nitrobenzene (12.7 g, 90%). MS-ESI: 250 [MNa]+

¹H NMR Spectrum: (CDCl₃) 2.38 (s, 3H); 4.0 (s, 3H); 4.25 (s, 2H); 7.0 (dd, 1H); 8.05 (d, 1H)

To a solution of 3-acetylmethyl-2-fluoro-1-methoxy-4-nitrobenzeene (11.36 g, 50 mmol) in acetone (200 ml) was added 4M aqueous ammonium acetate (700 ml) followed by a solution of titanium trichloride (15% in water, 340 ml) dropwise. The mixture was stirred for 10 minutes at ambient temperature and the mixture was extracted with ether. The organic layer was washed with 0.5N aqueous sodium hydroxide followed by water, brine, dried (MgSO₄) and the volatiles were removed under vacuum. The residue was purified by column chromatography eluting with methylene chloride to give 4-fluoro-5-methoxy-2-methylindole (8.15 g, 90%).

¹H NMR Spectrum: (DMSO) 2.35 (s, 3H); 3.8 (s, 3H); 6.1 (s, 1H); 6.85 (dd, 1H); 7.02 (d, 1H)

Cleavage of 4-fluoro-5-methoxy-2-methylindole with boron tribromide to give 4-fluoro-5-hydroxy-2-methylindole is described above.

### EXAMPLE 10

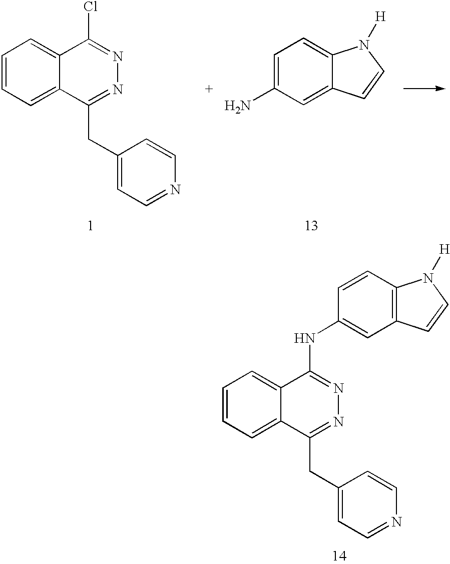

To a solution of 1-chloro-4-(4-pyridylmethyl)phthalazine (85 mg, 0.33 mmol), (J. Med. Chem. 2000, 43, 2310-2323), and 5-aminoindole (53 mg, 0.39 mmol) in isopropanol (5 ml) was added 5.5N HCl in isopropanol (70 μl). After stirring for 4 hours at 85° C., the solid was filtered, washed with isopropanol followed by ether and dried under vacuum to give 1-(indol-5-ylamino)-4-(4-pyridylmethyl)phthalazine hydrochloride (52 mg, 37%).

¹H NMR Spectrum: (DMSO d₆) 4.6 (s, 2H); 6.55 (s, 1H); 7.25 (dd, 1H); 7.42 (d, 2H); 7.48 (t, 1H); 7.58 (d, 1H); 7.78 (s, 1H); 8.18 (n, 2H); 8.3 (m, 1H); 8.52 (d, 2H); 9.0 (m, 1)

MS-ESI: 352 [MH]⁺

### EXAMPLE 11

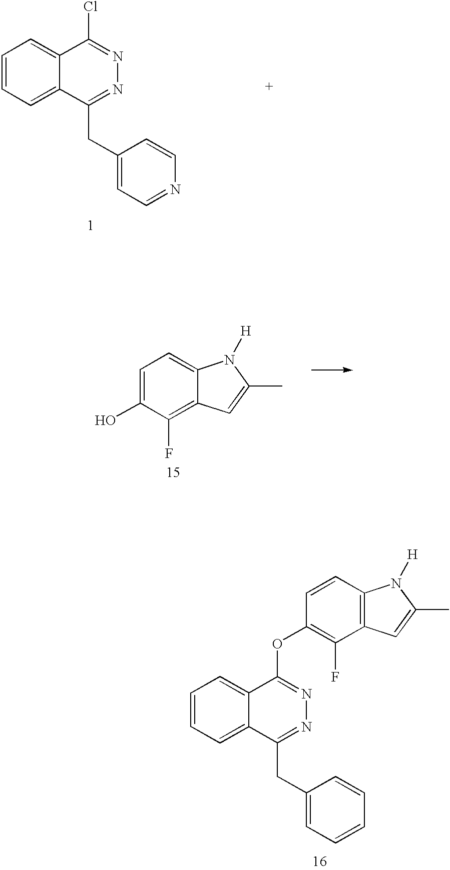

Under nitrogen a solution of 1-chloro-4-(4-pyridylmethyl)phthalazine (160 mg, 0.62 mmol), (J. Med. Chem. 2000,43,2310-2323), 4-fluoro-5-hydroxy-2-methylindole (124 mg, 0.75 mmol), (prepared as described for the starting material in Example 9), and cesium carbonate (408 mg, 1.2 mmol) in DMF (4 ml) was heated at 95° C. for 1.5 hours. After cooling, the mixture was filtered and the volatiles were removed under vacuum. The residue was purified by column chromatography, eluting with methylene chloride, followed by methylene chloride/ethyl acetate/methanol (45150/5). The fractions containing the expected product were combined and evaporated. The residue was triturated with ether, and the solid was filtered, washed with ether and dried under vacuum to give 1-(4-fluoro-2-methylindol-5-yloxy)-4-(4-pyridylmethyl)phthalazine (98 mg, 41%).

¹H NMR Spectrum: (CDCl₃) 2.42 (s, 3H); 4.61 (s, 2H); 6.28 (s, 1H); 7.02 (s, 1H); 7.04 (d, 1H); 7.24 (d, 2H); 7.8-8.0 (m, 3H); 8.3(brs, 1H); 8.49 (d, 2H); 8.54 (d, 1H)

MS-ESI: 385 [MH]⁺

### EXAMPLE 12

The following illustrate representative pharmaceutical dosage forms containing the compound of formula I, or a pharmaceutically acceptable salt thereof (hereafter compound X), for therapeutic or prophylactic use in humans:

| Tablet I | mg/tablet |
|---|---|
| Compound X | 100 |
| Lactose Ph.Eur | 182.75 |
| Croscarmellose sodium | 12.0 |
| Maize starch paste | 2.25 |
| (5% w/v paste) | |
| Magnesium stearate | 3.0 |

| Tablet II | mg/tablet |
|---|---|
| Compound X | 50 |
| Lactose Ph.Eur | 223.75 |
| Croscarmellose sodium | 6.0 |
| Maize starch | 15.0 |
| Polyvinylpyrrolidone | 2.25 |
| (5% w/v paste) | |
| Magnesium stearate | 3.0 |

| Tablet III | mg/tablet |
|---|---|
| Compound X | 1.0 |
| Lactose Ph.Eur | 93.25 |
| Croscarmellose sodium | 4.0 |
| Maize starch paste | 0.75 |
| (5% w/v paste) | |
| Magnesium stearate | 1.0 |
| Capsule | mg/capsule |
| Compound X | 10 |
| Lactose Ph.Eur | 488.5 |
| Magnesium stearate | 1.5 |
| Injection I | (50 mg/ml) |
| Compound X | 5.0% w/v |
| 1N Sodium hydroxide solution | 15.0% v/v |
| 0.1N Hydrochloric acid | |
| (to adjust pH to 7.6) | |
| Polyethylene glycol 400 | 4.5% w/v |
| Water for injection to 100% | |
| Injection II | 10 mg/ml) |
| Compound X | 1.0% w/v |
| Sodium phosphate BP | 3.6% w/v |
| 0.1N Sodium hydroxide solution | 15.0% v/v |
| Water for injection to 100% | |
| Injection III | (1 mg/ml, buffered to pH6) |
| Compound X | 0.1% w/v |
| Sodium phosphate BP | 2.26% w/v |
| Citric acid | 0.38% w/v |
| Polyethylene glycol 400 | 3.5% w/v |
| Water for injection to 100% | |

Note

The above formulations may be obtained by conventional procedures well known in the pharmaceutical art. The tablets (a)-(c) may be enteric coated by conventional means, for example to provide a coating of cellulose acetate phthalate.

### REFERENCE EXAMPLE 1

2-methyl-1H-pyrrolo[2,3-b]pyridin-5-ol

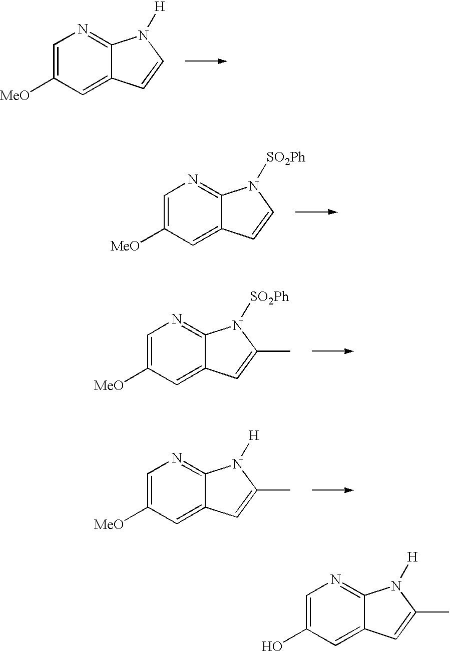

To a solution of 5-methoxy-1H-pyrrolo[2,3-b]pyridine (920 mg, 6.2 mmol) (Heterocycles 50, (2) 1065-1080, 1999) in methylene chloride (20 ml) was added benzyltriethylammonium chloride (37 mg, 0.16 mmol) followed by sodium hydroxide powder (771 mg, 19.2 mmol). The mixture was cooled to 0° C. and benzylsulfonyl chloride (991 μl, 7.77 mmol) was added dropwise. The mixture was stirred at 0° C. for 15 minutes followed by 2 hours at ambient temperature. The mixture was filtered over diatomaceous earth and the filtrate was evaporated under vacuum. The residue was purified by column chromatography eluting with ethyl acetate/petroleum ether (20/80 followed by 30/70). The fractions containing the expected product were combined and evaporated to give 5-methoxy-1-(phenylsulfonyl)-1H-pyrrolo[2,3-b]pyridine (1.69 g; 94%)

¹H NMR Spectrum: (DMSO d₆) 3.86 (s, 3H); 6.78 (d, 1H); 7.6-7.7 (m, 3); 7.72 (dd, 1H); 7.88 (d, 1H); 8.02-8.12 (m, 3H)

MS: 289.47 [M+H]+

A solution of 5-methoxy-1-(phenylsulfonyl)-1H-pyrrolo[2,3-b]pyridine (900 mg, 3.12 mmol) in THF (22.5 ml) was added dropwise to a solution of lithium diisopropylamide (prepared from iBu-Li (2.5M in hexane); 2.5 ml) and diisopropylamine (874 μl) in THF (13.5 ml)) cooled at −25° C. and the mixture was stirred for 30 minutes. Methyl iodide (215 μl, 3.44 mmol) in THF (9 ml) was then added dropwise and the mixture was stirred for 10 minutes at −25° C., left to warm up to ambient temperature and stirred for 15 minutes. The mixture was then poured onto ice/water. The mixture was then extracted with ethyl acetate. The organic layer was separated, washed with water, brine, dried (MgSO₄), filtered and evaporated. The residue was purified by column chromatography, eluting with ethyl acetate/petroleum ether (20/80 followed by 30/70). The fractions containing the expected product were combined and evaporated to give 5-methoxy-2-methyl-1-(phenylsulfonyl)-1H-pyrrolo[2,3-b]pyridine (805 mg, 85%).

¹H NMR Spectrum: (DMSOd₆) 2.7 (s, 3H); 3.82 (s, 3H); 6.51 (d, 1H); 7.49 (d, 1H); 7.59 (dd, 2H);7.7 (m, 1H); 8.0-8.1 (m, 3H)

MS: 303.5 [M+H]+

A solution of 5-methoxy-2-methyl-1-(phenylsulfonyl)-1H-pyrrolo[2,3-b]pyridine (950 mg, 3.14 mmol) and 40% aqueous sodium hydroxyde (106 ml) in methanol (160 ml) was heated at reflux for 30 minutes. After cooling, the mixture was poured onto cooled water and extracted with ethyl acetate. The organic layer was separated, washed with water, brine, dried (MgSO₄), filtered and evaporated. The residue was purified by column chromatography eluting with ethyl acetate/petroleum ether (1/1). The fractions containing the expected product were combined and evaporated to give 5-methoxy-2-methyl-1H-pyrrolo[2,3-b]pyridine (462 mg, 91%).

¹H NMR Spectrum: (DMSO d₆) 2.38 (s, 3H); 3.8 (s, 3H); 6.06 (d, 1H); 7.39 (d, 1H); 7.82 (d, 1H)

MS: 163.3 [M+H]+

A solution of boron tribromide (64 μl, 0.68 mmol) in methylene chloride (200 μl) was added to a solution of 5-methoxy-2-methyl-1H-pyrrolo[2,3-b]pyridine (50 mg, 0.308 mmol) in methylene chloride (4 ml) cooled at −30° C. The mixture was left to warm up to ambient temperature and further stirred for 3 hours. The mixture was poured onto ice. The pH was adjusted to 6.2 with 6N aqueous sodium hydroxide followed by 2 N aqueous hydrogen chloride. The mixture was extracted with ethyl acetate. The organic layer was washed with water, followed by brine and dried (MgSO₄), filtered and the filtrate was evaporated. The residue was purified by column chromatography, eluting with with methylene chloride followed by methylene chloride/methanol (98/2 followed by 95/5). The fractions containing the expected product were combined and evaporated to give 2-methyl-1H-pyrrolo[2,3-b]pyridin-5-ol (45 mg, quantitative).

¹H NMR Spectrum: (DMSO d₆) 2.4 (s, 3H); 5.96 (s, 1H); 7.12 (d, 1H); 7.69 (d, 1H); 8.9 (s, 1H); 11.07 (brs, 1H)

MS: 149.2 [M+H]+

1. The use of a compound of the formula I: 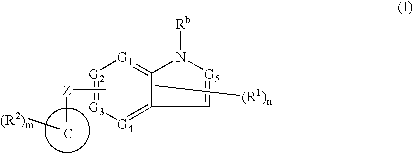
wherein:
ring C is a 9 or 10-membered bicyclic heteroaromatic group containing at least one nitrogen atom in the ring attached to Z and optionally containing a further 1-3 heteroatoms, selected independently from O, S and N, with the proviso that ring C is not a quinazoline, quinoline or cinnoline group;
either any one of G₁, G₂, G₃, G₄ and G₅ is nitrogen and the other four are —CH—, or G₁, G₂, G₃, G₄ and G₅ are all —CH—;
Z is —O—, —NH— or —S—; Z is linked to any one of G₁, G₂, G₃ and G₄ which is a free carbon atom;
n is an integer from 0 to 5; any of the substituents R¹ may be attached at any free carbon atom of the indole, azaindole or indazole group, such free carbon atoms may be G₁, G₂, G₃, G₄ or G₅ or may be at the 3-position of the indole, azaindole or indazole group;
m is an integer from 0 to 2;
R^{b} represents hydrogen, C₁₋₄alkyl, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, C₂₋₅alkenylaminoC₁₋₄alkyl, C₂₋₅alkynylaminoC₁₋₄alkyl, —C₁₋₅alkyl(ring A) wherein ring A is selected from azetidinyl, pyrrolidinyl, piperidinyl, piperazinyl, morpholino and thiomorpholino and wherein ring A may bear one or more substituents selected from C₁₋₄alkyl, C₂₋₅alkenyl, C₂₋₅alkynyl, hydroxy, oxo, halogeno, cyano, cyanoC₁₋₄alkyl, C₁₋₄alkylsulphonyl and C₁₋₄alkanoyl;
R¹ represents hydrogen, oxo, hydroxy, halogeno, C₁₋₄alkyl, C₁₋₄alkoxy, C₁₋₄alkoxyC₁₋₄alkyl, aminoC₁₋₄alkyl, C₁₋₃alkylaminoC₁₋₄alkyl, di(C₁₋₃alkyl)aminoC₁₋₄alkyl, —C₁₋₅alkyl(ring B) wherein ring B is selected from azetidinyl, pyrrolidinyl, piperidinyl, piperazinyl, N-methylpiperazinyl, N-ethylpiperazinyl, morpholino and thiomorpholino;
R² represents hydrogen, hydroxy, halogeno, cyano, nitro, trifluoromethyl, C₁₋₃alkyl, C₁₋₃alkoxy, C₁₋₃alkylsulphanyl, —NR³R⁴ (wherein R³ and R⁴, which may be the same or different, each represents hydrogen or C₁₋₃alkyl), or R⁵X¹— (wherein X¹ represents a direct bond, —O—, —CH₂—, —OC(O)—, —C(O)—, —S—, —SO—, —SO₂—, —NR⁶C(O)—, —C(O)NR⁷—, —SO₂NRW—, —NR⁹SO₂— or —NR¹⁰ (wherein R⁶, R⁷, R⁸, R⁹ and R¹⁰ each independently represents hydrogen, C₁₋₃alkyl or C₁₋₃alkoxyC₂₋₃alkyl), and R⁵ is selected from one of the following twenty-two groups:
1) hydrogen, oxiranylC₁₋₄alkyl or C₁₋₅alkyl which may be unsubstituted or which may be substituted with one or more groups selected from hydroxy, fluoro, chloro, bromo and amino;
2) C₁₋₅alkylX²C(O)R¹¹ (wherein X² represents —O— or NR¹² (in which R¹² represents hydrogen, C₁₋₃alkyl or C₁₋₃alkoxyC₂₋₃alkyl) and R¹¹ represents C₁₋₃alkyl, —NR¹³R¹⁴ or —OR¹⁵ (wherein R¹³, R¹⁴ and R¹⁵ which may be the same or different each represents hydrogen, C₁₋₅alkyl or C₁₋₃alkoxyC₂-3alkyl));
3) C₁₋₅alkylX³R¹⁶ (wherein X³ represents —O—, —S—, —SO—, —SO₂—, —OC(O)—, —NR¹⁷C(O)—, —C(O)NR¹⁸—, —SO₂NR¹⁹—, —NR²⁰SO₂— or —NR²¹— (wherein R¹⁷, R¹⁸, R¹⁹, R²⁰ and R²¹ each independently represents hydrogen, C₁₋₃alkyl or C₁₋₃alkoxyC₂₋₃alkyl) and R¹⁶ represents hydrogen, C₁₋₃alkyl, cyclopentyl, cyclohexyl or a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));
4) C₁₋₅alkylX⁴C₁₋₅alkylX⁵R²² (wherein X⁴ and X⁵ which may be the same or different are each —O—, —S—, —SO—, —SO₂—, —NR²³C(_)—, —C(O)NR²⁴—, SO₂NR²⁵—, —NR²⁶SO₂— or —NR²⁷— (wherein R²³, R²⁴, R²⁵, R²⁶ and R²⁷ each independently represents hydrogen, C₁₋₃alkyl or C₁₋₃alkoxyC₂₋₃alkyl) and R²² represents hydrogen, C₁₋₃alkyl or C₁₋₃alkoxyC₂₋₃alkyl);
5) R²⁸ (wherein R²⁸ is a 4-, 5- or 6-membered saturated heterocyclic group (linked via carbon or nitrogen) with 1-2 heteroatoms, selected independently from O, S and N, which heterocyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkanoyl, C₁₋₄alkoxyC₄alkyl, C₁₋₄alkylsulphonyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));
6) C₁₋₅alkylR²⁸ (wherein R²¹ is as defined herein);
7) C₂₋₅alkenylR²⁸ (wherein R²⁸ is as defined herein);
8) C₂₋₅alkynylR²⁸ (wherein R²⁸ is as defined herein);
9) R²⁹ (wherein R²⁹ represents a pyridone group, a phenyl group or a 5-6-membered aromatic heterocyclic group (linked via carbon or nitrogen) with 1-3 heteroatoms selected from O, N and S, which pyridone, phenyl or aromatic heterocyclic group may carry up to 5 substituents selected from hydroxy, halogeno, amino, C₁₋₄alkyl, C₁₋₄alkoxy, C₄hydroxyalkyl, C₁₋₄aminoalkyl, C₁₋₄alkylamino, C₁₋₄hydroxyalkoxy, carboxy, trifluoromethyl, cyano, —C(O)NR³⁰R³¹, —NR³²C(O)R³³ (wherein R³⁰, R³¹, R³² and R³³, which may be the same or different, each represents hydrogen, C₁₋₄alkyl or C₁₋₃alkoxyC₂₋₃alkyl) and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl));
10) C₁₋₅alkylR²⁹ (wherein R²⁹ is as defined herein);
11) C₂₋₅alkenylR²⁹ (wherein R²⁹ is as defined herein);
12) C₂₋₅alkynylR²⁹ (wherein R²⁹ is as defined herein);
13) C₁₋₅alkylR⁶R²⁹ (wherein X⁶ represents —O—, —S—, —SO—, —SO₂—, —NR³⁴C(O)—, —C(O)NR³⁵—, —SO₂NR³⁶—, —NR³⁷SO₂— or —NR³⁸— (wherein R³⁴, R³⁵, R³⁶, R³⁷ and R³⁸ each independently represents hydrogen, C₁₋₃alkyl or C₁₋₃alkoxyC₂₋₃alkyl) and R²⁹ is as defined herein);
14) C₂₋₅alkenylR⁷R²⁹ (wherein X⁷ represents —O—, —S—, —SO—, —SO₂—, —NR³⁹C(O)—, —C(O)NR⁴⁰—, SO₂NR⁴¹—, —NR⁴²SO₂— or —NR⁴³— (wherein R³⁹, R⁴⁰, R⁴¹, R⁴² and R⁴³ each independently represents hydrogen, C₁₋₃alkyl or C₁₋₃alkoxyC₂₋₃alkyl) and R²⁹ is as defined herein);
15) C₂₋₅alkynylX⁸R²⁹ (wherein X⁸ represents —O—, —S—, —SO—, SO—, —NR⁴⁴C(O)—, —C(O)NR⁴⁵, —SO₂NR⁴⁶—, —NR⁴⁷SO₂— or —NR⁴⁸— (wherein R⁴⁴, R⁴⁵, R⁴⁶, R⁴⁷ and R⁴⁸ each independently represents hydrogen, C₁₋₃alkyl or C₁₋₃alkoxyC₂₋₃alkyl) and R²⁹ is as defined herein);
16) C₁₋₄alkylR⁹C₁₋₄alkylR²⁹ (wherein X⁹ represents —O—, —S—, —SO—, —SO₂—, —NR⁴⁹C(O)—, —C(O)NR⁵⁰—, —SO₂NR⁵¹—, —NR⁵²SO₂ or —NR⁵³— (wherein R⁴⁹, R⁵⁰, R⁵¹, R⁵² and R⁵³ each independently represents hydrogen, C₁₋₃alkyl or C₁₋₃alkoxyC₂₋₃alkyl) and R²⁹ is as defined herein);
17) C₁₋₄alkylX⁹C₁₋₄alkylR²⁸ (wherein X⁹ and R²⁸ are as defined herein);
18) C₂₋₅alkenyl which may be unsubstituted or which may be substituted with one or more groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;
19) C₂₋₅alkynyl which may be unsubstituted or which may be substituted with one or more groups selected from hydroxy, fluoro, amino, C₁₋₄alkylamino, N,N-di(C₄alkylamino, aminosulphonyl, N-C₁₋₄alkylaminosulphonyl and N,N-di(C₁₋₄alkyl)aminosulphonyl;
20) C₂₋₅alkenylX⁹C₁₋₄alkylR²⁸ (wherein X9 and R²⁸ are as defined herein);
21) C₂ 5alkynylX⁹C₁₋₄alkylR²⁸ (wherein X⁹ and R²⁸ are as defined herein); and
22) C₁₋₄alkylR⁵⁴(C₄alkyl)_{q}(X⁹)ᵣR⁵⁵ (wherein X⁹ is as defined herein, q is 0 or 1, r is 0 or 1, and R⁵⁴ and R⁵⁵ are each independently selected from hydrogen, C₁₋₃alkyl, cyclopentyl, cyclohexyl and a 4-, 5- or 6-membered saturated heterocyclic group with 1-2 heteroatoms, selected independently from O, S and N, which C₁₋₃alkyl group may bear 1 or 2 substituents selected from oxo, hydroxy, halogeno and C₁₋₄alkoxy and which cyclic group may bear 1 or 2 substituents selected from oxo, hydroxy, halogeno, cyano, C₁₋₄cyanoalkyl, C₁₋₄alkyl, C₁₋₄hydroxyalkyl, C₁₋₄alkoxy, C₁₋₄alkoxyC₁₋₄alkyl, C₁₋₄alkylsulphonylC₁₋₄alkyl, C₁₋₄alkoxycarbonyl, C₁₋₄aminoalkyl, C₁₋₄alkylamino, di(C₁₋₄alkyl)amino, C₁₋₄alkylaminoC₁₋₄alkyl, di(C₁₋₄alkyl)aminoC₁₋₄alkyl, C₁₋₄alkylaminoC₁₋₄alkoxy, di(C₁₋₄alkyl)aminoC₁₋₄alkoxy and a group —(—O—)_{f}(C₁₋₄alkyl)_{g}ringD (wherein f is 0 or 1, g is 0 or 1 and ring D is a 4-, 5- or 6-membered saturated heterocyclic group with 1-2 heteroatoms, selected independently from O, S and N, which cyclic group may bear one or more substituents selected from C₁₋₄alkyl), with the proviso that R⁵⁴ cannot be hydrogen);
and additionally wherein any C₁₋₅alkyl, C₂₋₅alkenyl or C₂₋₅alkynyl group in R⁵X¹— may bear one or more substituents selected from hydroxy, halogeno and amino);
with the proviso that when ring C is 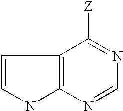
wherein Z is as defined herein, (but Z is not a part of ring C, it is shown for the purposes of clarity), at least one R² does not have a value selected from hydrogen, halogeno, C₁₋₄alkyl, C₁₋₄alkoxy and NR^{c}R^{d} (wherein each of R^{c} and R^{d} independently represents hydrogen, C₁₋₄alkyl or phenyl which phenyl may bear 1-3 substituents selected from halogeno, trifluoromethyl, C₁₋₄alkyl and C₁₋₄alkoxy);
or a salt thereof, in the manufacture of a medicament for use in the production of an antiangiogenic and/or vascular permeability reducing effect in warm-blooded animals such as humans.

2. The use of a compound according to claim 1 wherein the optionally substituted indole moiety of formula II: 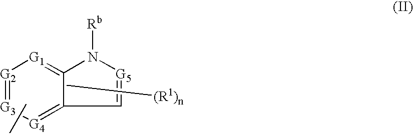
wherein R¹, R^{b}, G₁, G₂, G₃, G₄, G₅ and n are as defined in claim 1; is selected from the indole moieties:
4-fluoro-2-methylindol-5-yl, 2-methylindol-5-yl, 2-methylindol-6-yl, 2,3-dimethylindol-5-yl, 1-methylindol-5-yl, 1,2-dimethylindol-5-yl, 4-fluoroindol-5-yl, 6-fluoroindol-5-yl, indol-5-yl and 3-methylindol-5-yl.

3. A compound of the formula Ib: 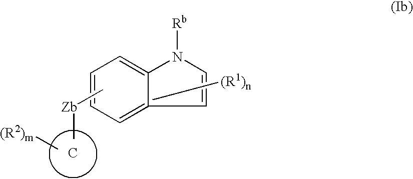
wherein ring C, R^{b}, R¹, R², m and n are as defined in claim 1 and Zb represents —O—, —NH— or —S—;
with the proviso that if Zb is —NH— then:
at least one R² is not selected from hydrogen, chloro, bromo, methyl, phenyl(hydroxymethyl), dimethylamino, methylsulphanyl, methylsulphinyl, methylsulphonyl and hydroxycyclohexylamino;
X¹ is not selected from —CH₂—, a direct bond and —C(O)NR⁷—, wherein R⁷ and X¹ are as defined in claim 1; and
where R² is a group R⁵—X¹ and X¹ is —NR⁶C(O)— or —NR⁹SO₂—, R⁵ does not contain an alkenyl or alkynyl moiety, wherein R⁵, R⁶, R⁹ and X¹ are as defined in claim 1;
and with the further proviso that when ring C is 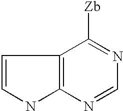
wherein Zb is as defined herein, (but Zb is not a part of ring C, it is shown for the purposes of clarity), at least one R² does not have a value selected from hydrogen, halogeno, C₁₋₄alkyl, C₁₋₄alkoxy and NR^{c}R^{d} (wherein each of R^{c} and R^{d} independently represents hydrogen, C₁₋₄alkyl or phenyl which phenyl may bear 1-3 substituents selected from halogeno, trifluoromethyl, C₁₋₄alkyl and C₁₋₄alkoxy);
or a salt thereof.

4. A compound according to claim 3 wherein ring C is selected from one of the following seven moieties: 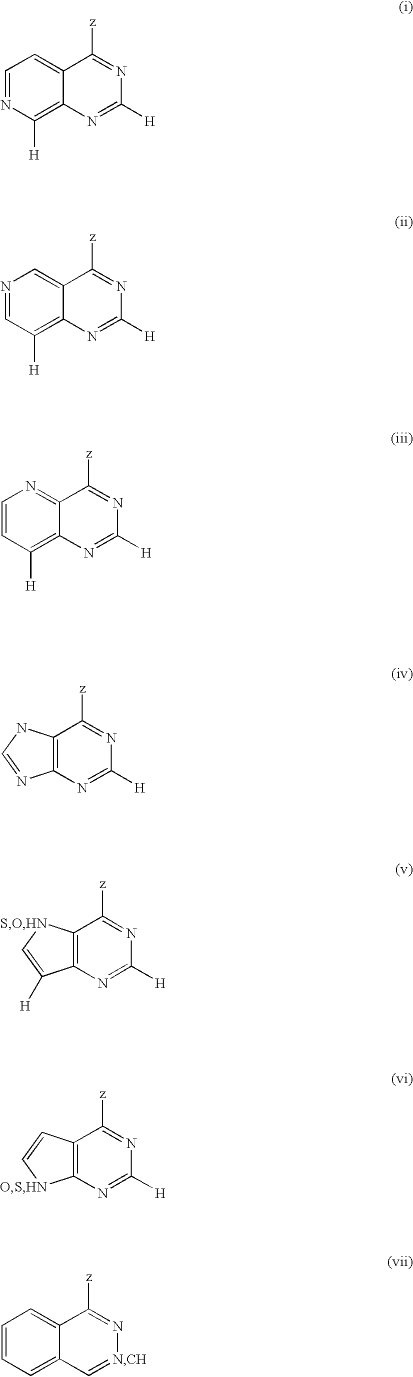
wherein Z is Zb as defined in claim 3 but is not part of ring C.

5. A compound according to claim 3 or claim 4 wherein ring C is a thienopyrimidine ring or a phthalazine ring.

6. A compound according to any one of claims 3 to 5 wherein Zb is —O— or —NH—.

7. A compound according to any one of claims 3 to 6 wherein R^{b} is hydrogen.

8. A compound according to any one of claims 3 to 7 wherein R¹ represents methyl, ethyl, trifluoromethyl or halogeno.

9. A compound according to any one of claims 3 to 8 wherein R² represents hydroxy, halogeno, nitro, trifluoromethyl, C₁₋₃alkyl, cyano, amino or R⁵—X¹— [wherein X¹ is as defined in claim 1 and R⁵ is selected from one of the following twenty groups:
1) C₁₋₃alkyl which may be unsubstituted or which may be substituted with one or more groups selected from fluoro, chloro and bromo, or C₂₋₃alkyl which may be unsubstituted or substituted with one or more groups selected from hydroxy and amino;
2) 2-(3,3-dimethylureido)ethyl, 3-(3,3-dimethylureido)propyl, 2-(3-methylureido)ethyl, 3-(3-methylureido)propyl, 2-ureidoethyl, 3-ureidopropyl, 2-(N,N-dimethylcarbamoyloxy)ethyl, 3-(N,N-dimethylcarbamoyloxy)propyl, 2-(E-methylcarbamoyloxy)ethyl, 3-(N-methylcarbamoyloxy)propyl, 2-(carbamoyloxy)ethyl, 3-(carbamoyloxy)propyl, or 2-(N-methyl-N-(butoxycarbonyl)amino)ethyl;
3) C₂₋₃alkylR³R⁶ (wherein X³ is as defined in claim 1 and R¹⁶ is a group selected from C₁₋₃alkyl, cyclopentyl, cyclohexyl, pyrrolidinyl, piperidinyl, piperazinyl, azetidinyl, imidazolidinyl and tetrahydropyranyl which group is linked to X³ through a carbon atom and which C₁₋₃alkyl group may bear 1 or 2 substituents selected from hydroxy, halogeno and C₁₋₂alkoxy and which cyclopentyl, cyclohexyl, pyrrolidinyl, piperidinyl, piperazinyl, azetidinyl, imidazolidinyl or tetrahydropyranyl group may bear one substituent selected from oxo, hydroxy, halogeno, cyano, C₁₋₂cyanoalkyl, C₁₋₂alkyl, C₁₋₂hydroxyalkyl, C₁₋₂alkoxy, C₁₋₂alkoxyC₁₋₃alkyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));
4) C₂₋₃alkylX⁴C₂₋₃alkylX⁵R²² (wherein X⁴ and X⁵ are as defined in claim 1 and R²² represents hydrogen or C₁₋₂alkyl);
5) R²⁸ (wherein R¹⁸ is as defined in claim 1);
6) C₁₋₃alkylR²⁹ (wherein R⁵⁹ is a group selected from pyrrolidinyl, piperazinyl, piperidinyl, azetidinyl, imidazolidinyl, 1,3-dioxolan-2-yl, 1,3-dioxan-2-yl, 1,3-dithiolan-2-yl and 1,3-dithian-2-yl, which group is linked to C₁₋₃alkyl through a carbon atom and which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino)) or C₂₋₃alkylR⁶⁰ (wherein R⁶⁰ is a group selected from morpholino, thiomorpholino, azetidin-1-yl, pyrrolidin-1-yl, piperazin-1-yl and piperidino which group may bear 1 or 2 substituents selected from oxo, hydroxy, halogeno, cyano, C₁₋₂cyanoalkyl, C₁₋₂alkyl, C₁₋₂hydroxyalkyl, C₁₋₂alkoxy, C₁₋₂alkanoyl, C₁₋₂alkoxyC₁₋₃alkyl, C₁₋₂alkylsulphonyl, C₁₋₂alkylsulphonylC₁₋₃alkyl, C₁₋₂alkoxycarbonyl, C₁₋₃alkylamino, di(C₁₋₃alkyl)amino, C₁₋₃alkylaminoC₁₋₃alkyl, di(C₁₋₃alkyl)aminoC₁₋₃alkyl, C₁₋₃alkylaminoC₁₋₃alkoxy, di(C₁₋₃alkyl)aminoC₁₋₃alkoxy and a group —(—O—)_{f}(C₁₋₃alkyl)_{g}ringD (wherein f is 0 or 1, g is 0 or 1 and ring D is a heterocyclic group selected from pyrrolidinyl, methylpiperazinyl, piperidinyl, azetidinyl, morpholino and thiomorpholino));
7) R²⁹ (wherein R²⁹ is as defined in claim 1);
8) C₁₋₄alkylR²⁹ (wherein R²⁹ is as defined in claim 1);
9) 1-R²⁹but-2-en-4-yl (wherein R²⁹ is as defined in claim 1);
10) 1-R²⁹but-2-yn-4-yl (wherein R²⁹ is as defined in claim 1);
11) C₁₋₃alkylX⁶R²⁹ (wherein X⁶ and R²⁹ are as defined in claim 1);
12) 1-(R²⁹X⁷)but-2-en-4-yl (wherein X⁷ and R²⁹ are as defined in claim 1);
13) 1-(R²⁹X⁸)but-2-yn-4-yl (wherein X⁸ and R²⁹ are as defined in claim 1);
14) C₂₋₃alkylX⁹C₁₋₃alkylR²⁹ (wherein X⁹ and R²⁹ are as defined in claim 1);
15) C₂₋₃alkylX⁹C₁₋₃alkylR²⁸ (wherein X⁹ and R²⁸ are as defined in claim 1);
16) C₂₋₅alkenyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;
17) C₂₋₅alkynyl which may be unsubstituted or which may be substituted with one or more fluorine atoms or with one or two groups selected from hydroxy, fluoro, amino, C₁₋₄alkylamino, N,N-di(C₁₋₄alkyl)amino, aminosulphonyl, N-C₁₋₄alkylaminosulphonyl and N,N-di(C₁₋₄alkyl)aminosulphonyl;
18) C₂₋₃alkenylX⁹C₁₋₃alkylR²⁸ (wherein X⁹ and R²⁸ are as defined in claim 1);
19) C₂₋₃alkynylX⁹C₁₋₃alkylR²⁸ (wherein X⁹ and R²⁸ are as defined in claim 1); and
20) C₁₋₃alkylR⁵⁴(C₁₋₃alkyl)_{q}(X⁹)ᵣR⁵⁵ (wherein X⁹, q, r, R⁵⁴ and R⁵⁵ are as defined in claim 1);
and additionally wherein any C₁₋₅alkyl, C₂₋₅alkenyl or C₂₋₅alkyl group in R⁵X¹— may bear one or more substituents selected from hydroxy, halogeno and amino].

10. A compound selected from:
1-(4-fluoroindol-5-yloxy)-4-(4-pyridylmethyl)phthalazine,
1-(indol-6-yloxy)-4-(4-pyridylmethyl)phthalazine,
1-(2-methylindol-6-yloxy)-4-(4-pyridylmethyl)phthalazine,
4-(4-fluoro-2-methylindol-5-yloxy)thieno[3,2-d]pyrimidine and
1-(4-fluoro-2-methylindol-5-yloxy)-4-(4-pyridylmethyl)phthalazine, or a salt thereof.

11. A compound according to any one of claims 3 to 10 in the form of a pharmaceutically acceptable salt.

12. A process for the preparation of a compound of formula Ib or a salt thereof which comprises:
(a) the reaction of a compound of the formula III: 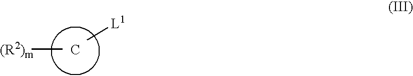
(wherein ring C, R² and m are as defined in claim 1 and L¹ is a displaceable moiety), with a compound of the formula IV: 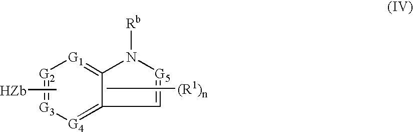
(wherein R^{b}, R¹ and n are as defined in claim 1, G₁, G₂, G₃, G₄ and G₅ are all —CH— and Zb is as defined in claim 3);
(b) a compound of formula Ib or a salt thereof wherein at least one R² is R⁵X¹ wherein R⁵ is as defined in claim 1 and X¹ is —O—, —S—, —OC(O)— or —NR¹⁰— (wherein R¹⁰ independently represents hydrogen, C₁₋₃alkyl or C₁₋₃alkoxyC₂₋₃alkyl) may be prepared by the reaction of a compound of the formula V: 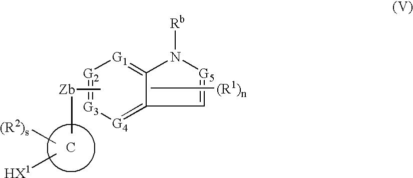
(wherein ring C, R^{b}, R¹, R² and n are as defined in claim 1, G, G₂, G₃, G₄ and G₅ are all —CH—, Zb is as defined in claim 3, X¹ is as herein defined in this section and s is 0 or 1) with a compound of formula VI:
R⁵-L¹ (VI)
(wherein R⁵ is as defined in claim 1 and L¹ is as defined herein);
(c) a compound of formula Ib or a salt thereof wherein at least one R¹ is R⁵X¹ wherein R⁵ is as defined in claim 1 and X¹ is —O—, —S—, —OC(O)— or —NR¹⁰— (wherein R¹⁰ represents hydrogen, C₁₋₃alkyl or C₁₋₃alkoxyC₂₋₃alkyl) may be prepared by the reaction of a compound of the formula VII: 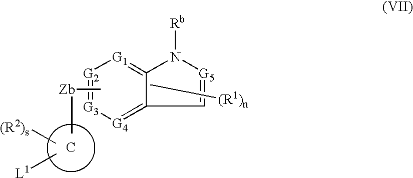
with a compound of the formula VIII:
R⁵—X¹—H (VIII)
(wherein ring C, R^{b}, R¹, R², R⁵ and n are all as defined in claim 1, G₁, G₂, G₃, G₄ and G₅ are all —CH—, Zb is as defined in claim 3, L¹ and s are as defined herein and X¹ is as herein defined in this section);
(d) a compound of formula Ib or a salt thereof wherein at least one R² is R⁵X¹ wherein X¹ is as defined in claim 1 and R⁵ is C₁₋₅alkylR⁶², wherein R⁶² is selected from one of the following nine groups:
1) X¹C₁₋₃alkyl (wherein X¹⁰ represents —O—, —S—, —SO₂—, —NR⁶³C(O)— or NR⁶⁴SO₂— (wherein R⁶³ and R⁶⁴ which may be the same or different are each hydrogen, C₁₋₃alkyl or C₁₋₃alkoxyC₂₋₃alkyl);
2) NR⁶⁵R⁶⁶ (wherein R⁶⁵ and R⁶⁶ which may be the same or different are each hydrogen, C₁₋₃alkyl or C₁₋₃alkoxyC₂₋₃alkyl);
3) X¹¹C₁₋₅alkylX⁵R²² (wherein X represents —O—, —S—, —SO₂—, —NR⁶⁷C(O)—, —NR⁶⁸SO₂— or —NR⁶⁹— (wherein R⁶⁷, R⁶⁸, and R⁶⁹ which may be the same or different are each hydrogen, C₁₋₃alkyl or C₁₋₃alkoxyC₂₋₃alkyl) and X⁵ and R²² are as defined in claim 1);
4) R²⁸ (wherein R²⁸ is as defined in claim 1);
5) X¹²R²⁹ (wherein X¹² represents —O—, —S—, —SO₂—, —NR⁷⁰C(O)—, —NR⁷¹SO₂—, or —NR⁷²— (wherein R⁷⁰, R⁷¹, and R⁷² which may be the same or different are each hydrogen, C₁₋₃alkyl or C₁₋₃alkoxyC₂₋₃alkyl) and R²⁹ is as defined in claim 1); and
6) X¹³C₁₋₃alkylR²⁹ (wherein X¹³ represents —O—, —S—, —SO₂—, —NR⁷³C(O)—, —NR⁷⁴SO₂— or —NR⁷⁵— -(wherein R⁷³, R⁷⁴ and R⁷⁵ each independently represents hydrogen, C₁₋₃alkyl or C₁₋₃alkoxyC₂₋₃alkyl) and R²⁹ is as defined in claim 1);
7) R²⁹ (wherein R²⁹ is as defined in claim 1);
8) X¹³C₁₋₄alkylR²⁸ (wherein X¹³ and R²⁸ are as defined in claim 1); and
9) R⁵⁴(C₁₋₄alkyl)_{q}(X⁹)ᵣR⁵⁵ (wherein q, r, X⁹, R⁵⁴ and R⁵⁵ are as defined in claim 1);
may be prepared by reacting a compound of the formula I: 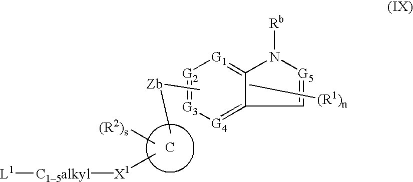
(wherein ring C, X¹, R^{b}, R¹, R² and n are as defined in claim 1, G₁, G₂, G₃, G₄ and G₅ are all —CH—, Zb is as defined in claim 3 and L¹, and s are as defined herein) with a compound of the formula X:
R⁶²—H (X)
(wherein R¹² is as defined herein);
(e) a compound of the formula Ib or a salt thereof wherein one or more of the substituents (R²)ₘ is represented by —NR⁷⁶R⁷⁷, where one (and the other is hydrogen) or both of R⁷⁶ and R⁷⁷ are C₁₋₃alkyl, may be effected by the reaction of compounds of formula I wherein the substituent (R²)ₘ is an amino group and an alkylating agent;
(f) a compound of the formula Ib or a salt thereof wherein X¹ is —SO— or —SO₂— may be prepared by oxidation from the corresponding compound in which X¹ is —S— or —SO— (when X¹ is —SO₂— is required in the final product);
and when a salt of a compound of formula Ib is required, reaction of the compound obtained with an acid or base whereby to obtain the desired salt.

13. A pharmaceutical composition which comprises a compound of the formula Ib as defined in claim 3 or a pharmaceutically acceptable salt thereof, in association with a pharmaceutically acceptable excipient or carrier.

14. A method for producing an antiangiogenic and/or vascular permeability reducing effect in a warm-blooded animal in need of such treatment which comprises administering to said animal an effective amount of a compound of formula I as defined in claim 1 or a pharmaceutically acceptable salt thereof.
